(19) United States
(12) Patent Application Publication
(10) Pub. No.: US 2022/0265709 A1
(43) Pub. Date: Aug. 25, 2022

(54) ENGINEERED CELLS AND USES THEREOF

(71) Applicant: NANJING LEGEND BIOTECH CO., LTD., Nanjing, Jiangsu Province (CN)

(72) Inventors: Huihui Zhang, Nanjing (CN); Fangliang Zhang, Nanjing (CN); Tao Zhao, Nanjing (CN); Ming Zeng, Irving, TX (US); Yafeng Zhang, Nanjing (CN); Wang Zhang, Nanjing (CN); Shu WU, Nanjing (CN); Qi Pan, Chappaqua, NY (US); Shuai Yang, Nanjing (CN)

(21) Appl. No.: 17/251,310

(22) PCT Filed: Jun. 19, 2019
(86) PCT No.: PCT/CN2019/091860

§ 371 Date: Dec. 17, 2020

(30) Foreign Application Priority Data
Jun. 19, 2018 (CN) PCTCN2018091789

(51) Int. Cl. A61K 35/17; C12N 5/0783; C12N 15/62; C12N 15/86; A61K 38/17; A61P 35/00

(52) U.S. Cl. CPC A61K 35/17; C12N 5/0636; C12N 15/625; C12N 15/86; A61K 38/1774; A61P 35/00; C12N 2740/15043; C12N 2501/2302

(57) ABSTRACT

A system for inducing activity of immune cells, comprises a chimeric antigen receptor, a T cell receptor, and various combinations thereof.

### CROSS-REFERENCE

This application claims priority to Patent Cooperation Treaty Application No. PCT/CN2018/091789, filed on Jun. 19, 2018, said application is incorporated herein by reference in its entirety for all purposes.

### BACKGROUND

Effector cell activities can involve a ligand binding to a membrane-bound receptor that comprises an extracellular antigen binding domain and an intracellular signaling domain. The formation of a complex between the antigen binding domain and its corresponding target can result in a conformational and/or chemical modification to the receptor itself, which in turn can yield an array of signals transduced within the cell. Attempts to harness this interaction for the development of immune cell therapies have shown promising efficacy but also off-target toxicity resulting in undesirable side effects, including cytokine release syndrome, in treated subjects. This and other side effects can further exuberate into inflammatory responses, organ failure, and, in extreme cases, death.

All T cell development and functions depend on its antigen receptor. The T cell receptor (TCR) is a multi-protein complex, comprised of two functionally different modules: a ligand binding module and a signal transmission module. The ligand-binding module is composed of two variable polypeptide chains, TCRα and TCRβ, which form a covalently linked heterodimer and are responsible for the ligand specificity of the TCR. The signal-transmission module of the TCR complex, is composed of invariant polypeptide chains, including CD3e, CD3g, CD3d, and z. Among them, CD3e, CD3g, and CD3d form non-covalently linked CD3eg and CD3ed heterodimers, whereas z forms a covalently linked zz homodimer. Surface expression of the TCR complex requires a fully assembled set of the complex subunits. Assembly begins with the formation, in the endoplasmic reticulum, of CD3ed and CD3eg heterodimers. These then associate with TCRα and TCRβ, respectively, to generate intermediate complexes. The zz homodimer is the last subunit to join, and upon its incorporation, the whole TCR complex is transported to the plasma membrane (Klausner et al., (1990); Exley et al., (1991); Dave et al., (1997); Marie-Cardine and Schraven, (1999); Kane et al., (2000); Matthew et al., (2004)).

pMHC binding to TCRαβ is transmitted into the cell via the CD3-signaling units, involving TCR-CD3 clustering and conformational changes. Many experiments have proved that T cell activation involves a cascade of TCR-mediated signals that are regulated by three distinct intracellular signaling motifs located within the cytoplasmic tails of the CD3 chains (CD3 zz, CD3eg, and CD3ed) (Sun et al, J Immunol (185), (2010). Studies using chimeric molecules have demonstrated that the cytoplasmic tails of all signaling chains of the TCR can independently transduce signals leading to cellular cytotoxicity and/or cytokine production, bypassing the αβ recognition modality of the TCR. However, based on experimental results, it was previous reported that signals through CD3 zeta chain alone are insufficient to prime resting T lymphocytes (Thomas et al, J. Exp. Med., (1995)), and mutated CD3e signaling domain in mice showed incomplete T cell function (Matthew et al, J Immunol (193), (2014).) The CD3eg, CD3ed and zz chains play a complementary role in contribute T cell functions, even synergy effect (Borroto et al, J Immunol (163), (1999)).

Chimeric antigen receptor (CAR) is a modular fusion protein comprising binding domain, spacer domain, transmembrane domain, and intracellular signaling domain containing CD3z linked with one or two costimulatory molecules. CAR structure has evolved significantly from the initial composition involving only the CD3ζ signaling domain, dubbed a “first-generation CAR.” Since then, in an effort to augment T-cell persistence and proliferation, costimulatory end domains were added, giving rise to second- (e.g., CD3ζ plus 4-1BB- or CD28-signaling domains) and third-generation (e.g., CD3ζ plus 4-1BB- and CD28-signaling domains) CARs.

The adoptive transfer of CAR T cells has demonstrated remarkable success in treating blood-borne tumors; prominently, the use of CD19 CARs in leukemias (Gill, S, et al, Blood Rev, (2015)), and indications in patients with lymphoma and myeloma are being explored. A growing number of clinical trials have focused on solid tumors. Unfortunately, the clinical results have been much less encouraging. To date, the two most positive trials reported have used GD2 CARs to target neuroblastoma (3 of 11 patients with complete remissions) (Louis et al, Blood (118), (2011)), and HER2 CARs for sarcoma (4 of 17 patients showing stable disease) (Ahmed et al, J Clin Oncol (33), (2015)).

It has been suggested that poor trafficking, limited persistence and T-cell inhibitory activity in patients' serum contributed to the observed lack of efficacy (Kershaw, et al. Clin. Cancer Res (12), (2006)). There are still unmet needs for new designs to improve the comprehensive functions of genetically modified T cells with better cell-killing effect, persistence in vivo and better tolerance to tumor microenvironments.

### SUMMARY

In view of the foregoing, there exists a considerable need for alternative compositions and methods to carry out immunotherapy. The compositions and methods of the present disclosure address this need, and provide additional advantages as well. The various aspects of the disclosure provide systems, compositions, and methods for inducing activity of immune cells.

In one aspect, provided is a system for inducing activity of an immune cell and/or a target cell, comprising: (a) a chimeric antigen receptor (CAR) comprising a first antigen binding domain which exhibits specific binding to a first epitope, a transmembrane domain, and an intracellular signaling domain; and (b) a modified T cell receptor (TCR) complex comprising a second antigen binding domain which exhibits specific binding to a second epitope, wherein said second antigen binding domain is linked to: (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), or (iii) a CD3 zeta chain.

In some embodiments, binding of the first antigen binding domain to the first epitope, and/or binding of the second antigen binding domain to the second epitope activates an immune cell activity of an immune cell expressing the system.

In some embodiments, two or more antigen binding domains are linked to, optionally in tandem, (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), (iii) a CD3 zeta chain, and wherein binding of the two more antigen binding domains to their respective epitopes activates an immune cell activity of an immune cell expressing the system.

In some embodiments, said immune cell activity is selected from the group consisting of: clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell.

In some embodiments, binding of the first antigen binding domain to the first epitope and binding of the second antigen binding domain to the second epitope activates cytotoxicity of an immune cell expressing the system, which cytotoxicity is enhanced as compared to binding of the first antigen binding domain to the first epitope alone, or binding of the second antigen binding domain to the second epitope alone.

In some embodiments, binding of the first antigen binding domain to the first epitope and binding of the second antigen binding domain to the second epitope activates cytotoxicity of an immune cell expressing the system and increases persistence of said cytotoxicity as compared to binding of the first antigen binding domain to the first epitope alone, or binding of the second antigen binding domain to the second epitope alone.

In some embodiments, binding of the two or more antigen binding domains to their respective epitopes activates cytotoxicity of an immune cell expressing the system and increases persistence of said cytotoxicity, as compared to binding of the first antigen binding domain to the first epitope alone, when said system is expressed in an immune cell in a subject.

In some embodiments, said modified TCR comprises a third antigen binding domain linked to: (i) said second antigen binding domain, (ii) the at least one TCR chain selected from an alpha chain, a beta chain, a gamma chain and a delta chain of a T cell receptor, (iii) the epsilon chain, the delta chain, and/or the gamma chain of cluster of differentiation 3 (CD3), or (iv) the CD3 zeta chain.

In some embodiments, said CAR comprises one or more additional antigen binding domains. In some embodiments, said one or more additional antigen binding domains exhibit specific binding to one or more additional epitopes. In some embodiments, said one or more additional epitopes are the same as the first or second epitope. In some embodiments, said one or more additional epitopes are different from the first and second epitope. In some embodiments, said one or more additional antigen binding domains and the first antigen binding domain are linked in tandem.

In some embodiments, said intracellular signaling domain of said CAR comprises an immunoreceptor tyrosine-based activation motif (ITAM). In some embodiments, said intracellular signaling domain of said CAR comprises an immunoreceptor tyrosine-based inhibition motif (ITIM).

In some embodiments, said intracellular signaling domain of said CAR comprises an signaling domain of an Fcγ receptor (FcγR), an Fcε receptor (FcεR), an Fcα receptor (FcαR), neonatal Fc receptor (FcRn), CD3, CD3 ζ, CD3 γ, CD3 δ, CD3 ε, CD4, CD5, CD8, CD21, CD22, CD28, CD32, CD40L (CD154), CD45, CD66d, CD79a, CD79b, CD80, CD86, CD278 (also known as ICOS), CD247 ζ, CD247 η, DAP10, DAP12, FYN, LAT, Lck, MAPK, MHC complex, NFAT, NF-κB, PLC-γ, iC3b, C3dg, C3d, and Zap70. In some embodiments, said intracellular signaling domain comprises a signaling domain of CD3 ζ.

In some embodiments, said CAR further comprises a co-stimulatory domain. In some embodiments, said co-stimulatory domain comprises a signaling domain of a MHC class I molecule, a TNF receptor protein, an immunoglobulin-like protein, a cytokine receptor, an integrin, a signaling lymphocytic activation molecule (SLAM protein), an activating NK cell receptor, or a Toll ligand receptor. In some embodiments, said co-stimulatory domain comprises a signaling domain of a molecule selected from: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF-R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8 α, CD8 β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6.

In some embodiments, said first antigen binding domain and/or said second antigen binding domain comprises a Fab, a Fab′, a F(ab′)₂, an Fv, a single-chain Fv (scFv), minibody, a diabody, a single-domain antibody, a light chain variable domain (VL), or a variable domain (V_{H}H) of camelid antibody.

In some embodiments, at least one of the antigen binding domains comprises a receptor. In some embodiments, at least one of the antigen binding domains comprises a ligand for a receptor.

In some embodiments, said first epitope and said second epitope are present on different antigens. In some embodiments, said first epitope and said second epitope are present on a common antigen.

In some embodiments, at least one epitope are present on one or more cell surface antigens. In some embodiments, said one or more cell surface antigens are tumor associated antigens, tyrosine kinase receptors, serine kinase receptors, and G-protein coupled receptors.

In some embodiments, said first epitope and/or said second epitope is present on a universal antigen.

In some embodiments, said first epitope and/or said second epitope is present on a neoantigen. In some embodiments, said first epitope and/or said second epitope is a neoepitope.

In some embodiments, said first epitope and/or said second epitope is present on a tumor-associated antigen. In some embodiments, the tumor-associated antigen is selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, ber-abl p190 (e1a2), ber-abl p210 (b2a2), ber-abl p210 (b3a2), BING-4, CA-125, CAG-3, CAIX, CAMEL, Caspase-8, CD171, CD19, CD20, CD22, CD4, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFαRII, TGFβRII, TP1, TRAG-3, TRG, TRP-1, TRP-2, TRP-2/INT2, TRP-2-6b, Tyrosinase, VEGF-R2, WT1, α-folate receptor, and κ-light chain.

In some embodiments, at least one epitope is present on an immune checkpoint receptor or immune checkpoint receptor ligand. In some embodiments, said immune checkpoint receptor or immune checkpoint receptor ligand is PD-1, PD-L1, PD-L2, CTLA-4, TIM-3, LAG3, TIGIT, BLTA, CD47 or CD40.

In some embodiments, at least one epitope is present on a cytokine or a cytokine receptor. In some embodiments, said cytokine or cytokine receptor is CCR2b, CXCR2 (CXCL1 receptor), CCR4 (CCL17 receptor), Gro-a, IL-2, IL-7, IL-15, IL-21, IL-12, Heparanase, CD137L, LEM, Bcl-2, CCL17, CCL19 or CCL2.

In some embodiments, at least one epitope is present on an antigen presented by a major histocompatibility complex (MHC). In some embodiments, the MHC is HLA class 1. In some embodiments, the MHC is HLA class 2.

In another aspect, provided is an isolated host cell expressing the system of the present disclosure.

In some embodiments, the host cell is an immune cell. In some embodiments, the immune cell is a lymphocyte. In some embodiments, the lymphocyte is a T cell. In some embodiments, the lymphocyte is a α/β T cell and/or γ/δ T cell. In some embodiments, the T cell is a CD8+T cell. In some embodiments, the T cell is a CD4+ T cell. In some embodiments, the lymphocyte is a natural killer (NK) cell.

In some embodiments, the host cell exhibits specific binding to two antigens simultaneously present in a target cell.

In another aspect, provided is an antigen-specific immune cell comprising at least two exogenously introduced antigen binding domains, one of which is linked to a T cell receptor (TCR) complex and another is linked to a chimeric antigen receptor (CAR), wherein the immune cell binds specifically to a target cell expressing one or more antigens recognized by the at least two exogenously introduced antigen binding domains.

In some embodiments, said antigen binding domain linked to the CAR primarily mediates interaction between the immune cell and the target cell, and the antigen binding domain linked to the TCR complex primarily mediates an immune cell activity when the interaction between the immune cell and the target cell takes place.

In some embodiments, said immune cell activity is selected from the group consisting of: clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell.

In some embodiments, said immune cell is a lymphocyte. In some embodiments, said lymphocyte is a T cell. In some embodiments, said lymphocyte is a α/β T cell and/or γ/δ T cell. In some embodiments, said T cell is a CD4+ T cell or a CD8+ T cell. In some embodiments, said lymphocyte is a natural killer (NK) cell. In some embodiments, two or more antigen binding domains are linked to, optionally in tandem, (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), (iii) a CD3 zeta chain.

In another aspect, provided is a population of immune cells, wherein individual immune cells expressing the system of the present disclosure, and said population of immune cells is characterized in that: upon exposing said population of immune cells to a target cell population in a subject, the population of immune cells induces death of at least 5% of the target cells within about 2 days.

In some embodiments, said population of immune cells comprises at most about 10¹¹ cells.

In some embodiments, said immune cells comprise lymphocytes. In some embodiments, the lymphocytes are T cells. In some embodiments, the lymphocytes are α/β T cells and/or γ/δ T cells. In some embodiments, the T cells are CD4+ T cells. In some embodiments, the T cells are CD8+ T cells. In some embodiments, the lymphocytes are natural killer (NK) cells.

In another aspect, provided is a method of inducing activity of an immune cell and/or a target cell, comprising: (a) expressing a system in an immune cell; and (b) contacting a target cell with the immune cell under conditions that induce said activity of the immune cell and/or the target cell, wherein the system expressed in the immune cell comprises: a chimeric antigen receptor (CAR) comprising a first antigen binding domain having binding specificity for a first epitope, a transmembrane domain, and an intracellular signaling domain; and a modified T cell receptor (TCR) complex comprising a second antigen binding domain linked to: (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), or (iii) a CD3 zeta chain.

In some embodiments, binding of the first antigen binding domain to the first epitope and/or binding of the second antigen binding domain to the second epitope activates cytotoxicity of the immune cell.

In some embodiments, two or more antigen binding domains are linked to, optionally in tandem, (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), (iii) a CD3 zeta chain.

In some embodiments, binding of the first antigen binding domain to the first epitope and binding of the second antigen binding domain to the second epitope activates cytotoxicity of the immune cell, which cytotoxicity is enhanced as compared to binding of the first antigen binding domain to the first epitope alone, or binding of the second antigen binding domain to the second epitope alone.

In some embodiments, binding of the first antigen binding domain to the first epitope or binding of the second antigen binding domain activates cytotoxicity of the immune cell and increases persistence of said cytotoxicity as compared to binding of the first antigen binding domain to the first epitope alone, or binding of the second antigen binding domain to the second epitope alone.

In some embodiments, cytotoxicity of the immune cell induces death of the target cell. In some embodiments, the target cell is a cancer cell. In some embodiments, the target cell is a hematopoietic cell. In some embodiments, the target cell is a solid tumor cell. In some embodiments, the target cell is a cell identified in one or more of heart, blood vessels, salivary gland, esophagus, stomach, liver, gallbladder, pancreas, intestine, colon, rectum, anus, endocrine gland, adrenal gland, kidney, ureter, bladder, lymph node, tonsils, adenoid, thymus, spleen, skin, muscle, brain, spinal cord, nerve, ovary, fallopian tube, uterus, vagina, mammary gland, testes, prostate, penis, pharynx, larynx, trachea, bronchi, lung, diaphragm, cartilage, ligaments, and tendon.

In some embodiments, said immune cell is a lymphocyte. In some embodiments, the lymphocyte is a T cell. In some embodiments, the lymphocyte is a α/β T cell and/or γ/δ T cell. In some embodiments, the T cell is a CD4+ T cell or CD8+ T cell. In some embodiments, the lymphocyte is a natural killer (NK) cell.

In some embodiments, binding of the two or more antigen binding domains to their respective epitopes activates cytotoxicity of an immune cell expressing the system and increases persistence of said cytotoxicity, as compared to binding of the first antigen binding domain to the first epitope alone, when said system is expressed in an immune cell in a subject.

In another aspect, provided is a composition comprising one or more polynucleotides that encodes: (a) a chimeric antigen receptor (CAR) comprising a first antigen binding domain having binding specificity for a first epitope, a transmembrane domain, and an intracellular signaling domain; and (b) a modified T cell receptor (TCR) complex comprising a second antigen binding domain which exhibits specific binding to a second epitope, wherein said second antigen binding domain is linked to: (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), or (iii) a CD3 zeta chain. In some embodiments, the one or more polynucleotides comprise a promoter operably linked thereto.

In another aspect, provided is a method of producing a modified immune cell, comprising genetically modifying the immune cell by expressing the composition of the present disclosure in said immune cell, thereby producing said modified immune cell.

In another aspect, provided is a method of treating a cancer of a subject, comprising: (a) administering to a subject an antigen-specific immune cell comprising a chimeric antigen receptor (CAR) comprising a first antigen binding domain and a modified T cell receptor (TCR) complex comprising a second antigen binding domain, wherein a target cell of a cancer of said subject expresses one or more antigens recognized by the first and/or second antigen binding domain, and wherein the immune cell binds specifically to the target cell, and (b) contacting the target cell with the antigen-specific immune cell via the first and/or second antigen binding domains under conditions that induces an immune cell activity of the immune cell against the target cell, thereby inducing death of the target cell of the cancer.

In another aspect, provided is a method of treating a cancer of a subject comprising: (a) administering to a subject an antigen-specific immune cell, wherein said antigen-specific immune cell is a genetically modified immune cell expressing the system of the present disclosure; and (b) contacting the target cell with the antigen-specific immune cell under conditions that induces an immune cell activity of the immune cell against a target cell of a cancer of said subject, thereby inducing death of the target cell of the cancer.

In some embodiments, the method further comprises genetically modifying an immune cell to yield said antigen-specific immune cell.

In some embodiments, said immune cell activity is selected from the group consisting of: clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell.

In some embodiments, said immune cell activity is cytotoxicity of the immune cell. In some embodiments, said cytotoxicity of the immune cell against the target cell yields at a least a 10% reduction in said cancer of said subject. In some embodiments, said immune cell activity is cytokine release by the immune cell. In some embodiments, a persistence of said immune cell activity is increased when both said first and second antigen binding domain bind their respective epitopes, as compared to binding of only the first antigen binding domain alone, or binding of the second antigen binding domain alone.

In some embodiments, said cancer is selected from: bladder cancer, bone cancer, brain cancer, breast cancer, cervical cancer, colon cancer, esophageal cancer, gastric cancer, glioma, head and neck cancer, kidney cancer, leukemia, acute myeloid leukemia (AML), multiple myeloma, liver cancer, lung cancer, lymphoma, melanoma, mesothelioma, medulloblastoma, ovarian cancer, pancreatic cancer, prostate cancer, rectal cancer, skin cancer, testicular cancer, tracheal cancer, and vulvar cancer.

In some embodiments, said immune cell is a lymphocyte. In some embodiments, the lymphocyte is a T cell. In some embodiments, the lymphocyte is a α/β T cell and/or γ/δ T cell. In some embodiments, the T cell is a CD4+ T cell. In some embodiments, the T cell is a CD8+ T cell. In some embodiments, the lymphocyte is a natural killer (NK) cell.

In another aspect, provided is an antigen binding molecule having the formula of A-X-B-Y-C-Z-D, wherein A comprises a sequence having at least 80% or 90% identity to any one selected from the group consisting of SEQ ID NOs: 47-56; B comprises a sequence having at least 80% or 90% identity to any one selected from the group consisting of SEQ ID NOs: 57-66; C comprises a sequence having at least 80% or 90% identity to any one selected from the group consisting of SEQ ID NOs: 67-76; D comprises a sequence having at least 80% or 90% identity to any one selected from the group consisting of SEQ ID NOs: 77-86; X comprises a sequence having at least 80% or 90% identity to any one selected from the group consisting of SEQ ID NOs: 87-96; Y comprises a sequence having at least 80% or 90% identity to any one selected from the group consisting of SEQ ID NOs: 97-106; and Z comprises a sequence having at least 80% or 90% identity to any one selected from the group consisting of SEQ ID NOs: 107-116.

In some embodiments, the antigen binding molecule exhibits a binding affinity (K_{D}) for human BCMA of 100 nm, 90 nm, 80 nm, 70 nm, 60 nm, 50 nm, 40 nm, 30 nm, 20 nm, 10 nm, or 1 nm or less as determined by surface plasmon resonance at 37° C.

In some embodiments, the antigen binding molecule comprises a sequence having at least 80% or 90% identity to any one selected from the group consisting of SEQ ID NOs: 14-23. In some embodiments, the antigen binding molecule comprises a sequence selected from the group consisting of SEQ ID NOs: 14-23.

In another aspect, provided is a modified T cell receptor (TCR) complex comprising one or more antigen binding domains, wherein said one or more antigen binding domains are linked to: (iv) at least one TCR chain selected from an alpha chain, a beta chain, a gamma chain and a delta chain of a T cell receptor; (v) an epsilon chain, a delta chain, and/or a gamma chain of cluster of differentiation 3 (CD3); or (vi) a CD3 zeta chain; and wherein at least one of the one or more antigen binding domains comprises an antigen binding molecule of the present disclosure.

In some embodiments, at least one or two of the one or more antigen binding domains comprises a sequence having at least 80% or 90% identity to any one selected from the group consisting of SEQ ID NOs: 3-23, and 38-46.

In some embodiments, the modified TCR complex comprises two or more antigen binding domains. In some embodiments, the two or more antigen binding domains are linked to separate chains of the TCR complex. In some embodiments, the two or more antigen binding domains are linked to one chain of the TCR complex. In some embodiments, the two or more antigen binding domains are linked in tandem to one chain of the TCR complex. In some embodiments, the modified TCR complex further comprises one or more antigen binding domains linked to another chain of the TCR complex.

In another aspect, provided is a modified T cell receptor (TCR) complex comprising two or more antigen binding domains exhibiting specific binding to two or more epitopes, wherein said two or more antigen binding domains are linked to: (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain.

In some embodiments, the two or more antigen binding domains are linked to separate chains of the TCR complex. In some embodiments, the two or more antigen binding domains are linked to one chain of the TCR complex. In some embodiments, the two or more antigen binding domains are linked in tandem to one chain of the TCR complex.

In some embodiments, the modified TCR complex further comprises one or more antigen binding domains linked to another chain of the TCR complex.

In some embodiments, the two or more antigen binding domains bind to BCMA. In some embodiments, the two or more antigen domains bind to the same epitope of BCMA. In some embodiments, the two or more antigen binding domains are anti-BCMA sdAbs. In some embodiments, the two or more antigen binding domains are selected from the sequences having at least 80% sequence identity to any one of SEQ ID NOs: 3-23.

In some embodiments, the two or more antigen binding domains are linked in tandem on the epsilon chain, the delta chain, and/or the gamma chain of cluster of differentiation 3 (CD3).

In another aspect, provided is an antigen-specific immune cell comprising the modified TCR complex of the present disclosure.

In some embodiments, the antigen-specific immune cell further comprises a chimeric antigen receptor (CAR) comprising one or more antigen binding domains exhibiting specific binding to their respective epitopes, a transmembrane domain, and an intracellular signaling domain. In some embodiments, the one or more antigen binding domains of CAR are arranged in tandem.

In some embodiments, the antigen-specific immune cell further comprises two or more chimeric antigen receptors (CARs) each comprising one or more antigen binding domains exhibiting specific binding to their respective epitopes, a transmembrane domain, and an intracellular signaling domain.

The method disclosed herein find utility in treating a wide variety of cancer including but not limited to: the cancer is selected from: bladder cancer, bone cancer, brain cancer, breast cancer, cervical cancer, colon cancer, esophageal cancer, gastric cancer, glioma, head and neck cancer, kidney cancer, leukemia, acute myeloid leukemia (AML), multiple myeloma, liver cancer, lung cancer, lymphoma, melanoma, mesothelioma, medulloblastoma, ovarian cancer, pancreatic cancer, prostate cancer, rectal cancer, skin cancer, testicular cancer, tracheal cancer, and vulvar cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of a CAR-TCR-T system comprising antigen-binding domains, as shown in the black and white striped oval and black oval, capable of binding an antigen, for example a tumor-associated antigen.

FIG. 2A shows a modified TCR complex comprising an antigen binding domain fused to an epsilon chain. FIG. 2B shows a modified TCR complex comprising an antigen binding domain fused to a delta chain. FIG. 2C shows a modified TCR complex comprising an antigen binding domain fused to a gamma chain. FIG. 2D shows a modified TCR complex comprising an antigen binding domain fused to an alpha chain. FIG. 2E shows a modified TCR complex comprising an antigen binding domain fused to a beta chain. FIG. 2F shows a modified TCR complex comprising two different antigen binding domains. A first antigen binding domain is fused to a first epsilon chain and a second antigen binding domain is fused to a second epsilon chain. FIG. 2G shows a modified TCR complex comprising two different antigen binding domains. A first antigen binding domain is fused to an epsilon chain and a second antigen binding domain is fused to a gamma chain. FIG. 2H shows a modified TCR complex comprising a first antigen binding domain fused to a second antigen binding domain, which in turn in fused to an epsilon chain. FIG. 2I shows a modified TCR complex comprising two different antigen binding domains. A first antigen binding domain is fused to an alpha chain and a second antigen binding domain is fused to a beta chain. FIG. 2J shows a modified TCR complex comprising two identical antigen binding domains. A first antigen binding domain is fused to an alpha chain and a second antigen binding domain is fused to a beta chain. FIG. 2K shows a modified TCR complex comprising a first antigen binding domain fused to a second antigen binding domain which in turn in fused to a delta chain. FIG. 2L shows a modified TCR complex comprising a first antigen binding domain fused to a second antigen binding domain which in turn in fused to a gamma chain. FIG. 2M shows a modified TCR complex comprising a first antigen binding domain fused to a second antigen binding domain which in turn in fused to an alpha chain. FIG. 2N shows a modified TCR complex comprising a TCR comprising a first antigen binding domain fused to a second antigen binding domain which in turn in fused to a beta chain. FIG. 2O shows a modified TCR complex comprising two different antigen binding domains. A first antigen binding domain is fused to an epsilon chain and a second antigen binding domain is fused to a delta chain. FIG. 2P shows a modified TCR complex comprising two different antigen binding domains. A first antigen binding domain is fused to a delta chain and a second antigen binding domain is fused to a gamma chain. FIG. 2Q shows a modified TCR complex comprising two different antigen binding domains. A first antigen binding domain is fused to an alpha chain and a second antigen binding domain is fused to an epsilon chain. FIG. 2R shows a modified TCR complex comprising two different antigen binding domains. A first antigen binding domain is fused to a beta chain and a second antigen binding domain is fused to an epsilon chain. FIG. 2S shows a modified TCR complex comprising two different antigen binding domains. A first antigen binding domain is fused to an alpha chain and a second antigen binding domain is fused to a gamma chain. FIG. 2T shows a modified TCR complex comprising two different antigen binding domains. A first antigen binding domain is fused to a beta chain and a second antigen binding domain is fused to a gamma chain. FIG. 2U shows a modified TCR complex comprising two different antigen binding domains. A first antigen binding domain is fused to an alpha chain and a second antigen binding domain is fused to a delta chain. FIG. 2V shows a modified TCR complex comprising two different antigen binding domains. A first antigen binding domain is fused to a beta chain and a second antigen binding domain is fused to a delta chain.

FIG. 3 shows a CAR comprising an antigen binding domain fused to a transmembrane domain and an intracellular signaling domain (e.g., CD3-zeta signaling chain).

FIG. 4A shows a modified TCR complex comprising a first antigen binding domain fused to a second antigen binding domain which is in turn fused to an epsilon chain. FIG. 4B shows a modified TCR complex comprising a first antigen binding domain fused to a second antigen binding domain which is in turn fused to a delta chain. FIG. 4C shows a modified TCR complex comprising a first antigen binding domain fused to a second antigen binding domain which is in turn fused to a gamma chain. FIG. 4D shows a modified TCR complex comprising a first antigen binding domain fused to a second antigen binding domain fused to a third antigen binding domain which is in turn fused to an epsilon chain. FIG. 4E shows a modified TCR complex comprising a first antigen binding domain fused to a second antigen binding domain fused to a third antigen binding domain which is in turn fused to an delta chain. FIG. 4F shows a modified TCR complex comprising a first antigen binding domain fused to a second antigen binding domain fused to a third antigen binding domain which is in turn fused to an gamma chain. FIG. 4G shows a modified TCR complex comprising a first antigen binding domain fused to a second antigen binding domain which is in turn fused to an epsilon chain and also comprises a third antigen binding domain fused to a fourth antigen binding domain which is in turn fused to an delta chain. FIG. 4H shows a modified TCR complex comprising a first antigen binding domain fused to a second antigen binding domain which is in turn fused to an epsilon chain and also comprises a third antigen binding domain fused to a fourth antigen binding domain which is in turn fused to a gamma chain. FIG. 4I shows a modified TCR complex comprising a first antigen binding domain fused to a second antigen binding domain which is in turn fused to a delta chain and also comprises a third antigen binding domain fused to a fourth antigen binding domain which is in turn fused to a gamma chain. FIG. 4J shows a modified TCR complex comprising a first antigen binding domain fused to a second antigen binding domain which is in turn fused to an epsilon chain and also comprises a third antigen binding domain fused to a gamma chain. FIG. 4K shows a modified TCR complex comprising a first antigen binding domain fused to a second antigen binding domain fused to a third antigen binding domain which is in turn fused to an epsilon chain and also comprises the fourth antigen binding domain fused to the fifth antigen binding domain fused to the sixth antigen binding domain which is in turn fused to a delta chain. FIG. 4L shows a modified TCR complex comprising a first antigen binding domain fused to a second antigen binding domain fused to a third antigen binding domain which is in turn fused to an epsilon chain and also comprises a fourth antigen binding domain fused to a fifth antigen binding domain fused to a sixth antigen binding domain which is in turn fused to a gamma chain. FIG. 4M shows a modified TCR complex comprising a first antigen binding domain fused to a second antigen binding domain fused to a third antigen binding domain which is in turn fused to a delta chain and also comprises a fourth antigen binding domain fused to a fifth antigen binding domain fused to a sixth antigen binding domain which is in turn fused to a gamma chain.

FIG. 5A shows a modified TCR complex comprising a first antigen binding domain fused to an epsilon chain and a CAR comprising a second antigen binding domain fused to a transmembrane domain and an intracellular signaling domain (e.g., CD3-zeta signaling chain). FIG. 5B shows a modified TCR complex comprising a first antigen binding domain fused to an delta chain and a CAR comprising a second antigen binding domain fused to a transmembrane domain and an intracellular signaling domain (e.g., CD3-zeta signaling chain). FIG. 5C shows a modified TCR complex comprising a first antigen binding domain fused to a gamma chain and a CAR comprising a second antigen binding domain fused to a transmembrane domain and an intracellular signaling domain (e.g., CD3-zeta signaling chain). FIG. 5D shows a modified TCR complex comprising a first antigen binding domain fused to an epsilon chain, a second antigen binding domain fused to a delta chain, and a CAR comprising a third antigen binding domain fused to a transmembrane domain and an intracellular signaling domain (e.g., CD3-zeta signaling chain). FIG. 5E shows a modified TCR complex comprising a first antigen binding domain fused to an epsilon chain, a second antigen binding domain fused to a gamma chain and a CAR comprising a third antigen binding domain fused to a transmembrane domain and an intracellular signaling domain (e.g., CD3-zeta signaling chain). FIG. 5F shows a modified TCR complex comprising a first antigen binding domain fused to a delta chain, a second antigen binding domain fused to a gamma chain, and a CAR comprising a third antigen binding domain fused to a transmembrane domain and an intracellular signaling domain (e.g., CD3-zeta signaling chain). FIG. 5G shows a modified TCR complex comprising a first antigen binding domain fused to a second antigen binding domain which is in turn fused to an epsilon chain and a CAR comprising a third antigen binding domain fused to a transmembrane domain and an intracellular signaling domain (e.g., CD3-zeta signaling chain). FIG. 5H shows a modified TCR complex comprising a first antigen binding domain fused to an epsilon chain and a CAR comprising a second antigen binding domain fused to a third antigen binding domain fused to a transmembrane domain and an intracellular signaling domain (e.g., CD3-zeta signaling chain).

FIG. 6A shows a vector construct of an anti-BCMA epsilon TCR. FIG. 6B shows a vector construct of an anti-BCMA-4-1BB-CD3zeta CAR. FIG. 6C shows a vector construct of an anti-BCMA or CD19-epsilon TCR and anti-CD19 or BCMA-4-1BB-CD3zeta CAR. FIG. 6D shows a vector construct of an anti-BCMA or CD19 gamma or delta TCR and anti-CD19 or BCMA-4-1BB-CD3zeta CAR. FIG. 6E shows a vector construct of a tandem anti-BCMA epsilon TCR. FIG. 6F shows a vector construct of a tandem anti-BCMA epsilon TCR and tandem anti-CD19 or BCMA-4-1BB-CD3zeta CAR.

FIG. 7 shows the CD19 and BCMA expression levels on different tumor cells and engineered cell lines.

FIG. 8A, FIG. 8B and FIG. 8C show the results of cytotoxicity assay, on day 3 or 6 post transduction, where anti-BCMA antibody (BCMA1-12) was fused to epsilon-TCR, at effector-to-target cell ratios (E:T) of 0.5:1, 1.5:1 and 3:1. FIG. 8D, FIG. 8E and FIG. 8F show the amounts of IFNγ in supernatant collected from the cytotoxicity assay of FIG. 8A, FIG. 8B and FIG. 8C by using HTRF.

FIG. 9 shows the result of cytotoxicity assay, on day 6 post transfection, where anti-BCMA system: anti-BCMA3-epsilon-TCR (BCMA3 eTCR), anti-BCMA2-epsilon-TCR (BCMA2 eTCR), anti-BCMA2-anti-BCMA3-epsilon TCR (tandem BCMA2&3 eTCR), and anti-BCMA1-anti-BCMA2-anti-BCMA3-gamma TCR (tandem BCMA1&2&3 gTCR), as well as control untransduced cells were co-cultured with RPMI-8226 cells (BCMA+) at an effector-to-target cell ratios (E:T) of 0.33:1.

FIG. 10 shows the result of cytotoxicity assay, on day 6 post transfection, where anti-BCMA systems, anti-BCMA2-anti-BCMA3 epsilon-TCR (Tandem BCMA 2-3 eTCR), anti-BCMA4-anti-BCMA5 epsilon-TCR (Tandem BCMA 4-5 eTCR), anti-BCMA2-anti-BCMA3-anti-BCMA4 epsilon-TCR (Tandem BCMA 2-3-4 eTCR), as well as control untransduced cells were co-cultured with CHO/BCMA/CD19 cells (BCMA+CD19+) at effector-to-target cell ratios (E:T) of 1.5:1 and 0.5:1.

FIG. 11A shows the result of cytotoxicity assay, on day 11 post transfection, where anti-BCMA and/or anti-CD19 systems: anti-BCMA1 epsilon-TCR (BCMA1 eTCR), anti-BCMA1 4-1BB-CD3zeta-CAR (BCMA1 BBzCAR), anti-CD19 epsilon-TCR (CD19 eTCR), and anti-BCMA1-anti-CD19-epsilon TCR (tandem BCMA1/CD19 eTCR), as well as control untransduced cells were co-cultured with NCI-H929 cells (BCMA+) at effector-to-target cell ratios (E:T) of 10:1 and 5:1. FIG. 11B shows the amount of IFNγ in supernatant collected from the cytotoxicity assay of FIG. 11A by using HTRF.

FIG. 12A shows the result of cytotoxicity assay, on day 6 post transfection, where anti-BCMA and/or anti-CD19 systems: anti-BCMA1-epsilon-TCR (BCMA eTCR), anti-BCMA1-4-1BB-CD3zeta-CAR (BCMA BBzCAR), anti-CD19-4-1BB-CD3zeta CAR (CD19 BBzCAR), anti-CD19-epsilon TCR (CD19 eTCR), anti-CD19-epsilon TCR/anti-BCMA1-4-1BB-CD3zeta CAR (CD19 eTCR/BCMA BBzCAR), and anti-BCMA1-epsilon TCR/anti-CD19-4-1BB-CD3 zeta CAR (BCMA eTCR/CD19 BBzCAR), as well as control untransduced cells were co-cultured with CHO/BCMA/CD19 cells (BCMA+ and CD19+) at effector-to-target cell ratios (E:T) of 20:1, 10:1, and 5:1. FIG. 12B shows the amount of IFNγ in supernatant collected from the cytotoxicity assay of FIG. 12A by using HTRF.

FIG. 13A shows the result of cytotoxicity assay, on day 5 post transfection, where anti-BCMA and/or anti-CD19 systems: anti-CD19 epsilon-TCR(CD19 eTCR), anti-BCMA1-anti-CD19-epsilon TCR (tandem BCMA1/CD19 eTCR), anti-CD19-epsilon TCR/anti-BCMA1-delta TCR (CD19 eTCR/BCMA1 dTCR), anti-CD19-epsilon TCR/anti-BCMA1-4-1BB-CD3zeta CAR (CD19 eTCR/BCMA1 BBzCAR), and anti-BCMA1-epsilon TCR/anti-CD19-4-1BB-CD3zeta CAR (BCMA1 eTCR/CD19 BBzCAR), as well as control untransduced cells were co-cultured with CHO-BCMA-CD19 cells (BCMA+ and CD19+) at effector-to-target cell ratios (E:T) of 10:1 and 5:1. FIG. 13B shows the result of cytotoxicity assay, on day 5 post transfection, where anti-BCMA and/or anti-CD19 systems: anti-BCMA1-epsilon TCR (BCMA1 eTCR), anti-BCMA1-4-1BB-CD3zeta CAR (BCMA1 BBzCAR), anti-BCMA1/anti-CD19-epsilon TCR (tandem BCMA1/CD19 eTCR), anti-CD19-epsilon TCR/anti-BCMA1-4-1BB-CD3 zeta CAR (CD19 eTCR/BCMA1 BBzCAR), and anti-BCMA1-epsilon TCR/anti-CD19-4-1BB-CD3 zeta CAR (BCMA1 eTCR/CD19 BBzCAR), as well as control untransduced cells were co-cultured with NCI-H929 cells (BCMA+) at effector-to-target cell ratios (E:T) of 2.5:1, and 5:1.

FIG. 14A shows the result of cytotoxicity assay, on day 6 post transfection, where anti-BCMA and/or anti-CD19 systems: anti-BCMA1 epsilon-TCR (BCMA1 eTCR), anti-CD19-epsilon TCR/anti-BCMA1-gamma TCR (CD19 eTCR/BCMA1 gTCR), anti-CD19-epsilon TCR/anti-BCMA1-delta TCR (CD19 eTCR/BCMA1 dTCR), anti-CD19-epsilon TCR/anti-BCMA1-4-1BB-CD3zeta CAR (CD19 eTCR/BCMA1 BBzCAR), as well as control untransduced cells were co-cultured with CHO-BCMA1-CD19 cells (BCMA+ and CD19+) at effector-to-target cell ratios (E:T) of 1.3:1. FIG. 14B and FIG. 14C show the amounts of IFNγ and TNFα in supernatant collected from the cytotoxicity assay of FIG. 14A by using HTRF.

FIG. 15A shows the result of cytotoxicity assay, on day 6 post transfection, where anti-BCMA and/or anti-CD19 systems: anti-BCMA epsilon-TCR (BCMA eTCR), anti-CD19-epsilon TCR/anti-BCMA-4-1BB-CD3zeta CAR (CD19 eTCR/BCMA BBzCAR), anti-BCMA and anti-CD19-epsilon-TCR (Tandem BCMA/CD19 dTCR), as well as control untransduced cells were co-cultured with CHO-BCMA-CD19 cells (BCMA+ and CD19+) at effector-to-target cell ratios (E:T) of 1.3:1. FIG. 15B and FIG. 15C show the amounts of IFNγ and TNFα in supernatant collected from the cytotoxicity assay of FIG. 15A by using HTRF.

FIG. 16A shows the result of cytotoxicity assay, on day 4 post transfection, where anti-BCMA system: anti-BCMA2 epsilon-TCR (BCMA2 eTCR), anti-BCMA2-epsilon TCR/anti-BCMA3-4-1BB-CD3zeta CAR (BCMA2 eTCR/BCMA3 BBzCAR), anti-BCMA2-gamma TCR/anti-BCMA3-4-1BB-CD3zeta CAR (BCMA2 gTCR/BCMA3 BBzCAR), anti-BCMA2-delta TCR/anti-BCMA3-4-1BB-CD3zeta CAR (BCMA2 dTCR/BCMA3 BBzCAR), as well as control untransduced cells were co-cultured with RPMI-8226 cells (BCMA+) at effector-to-target cell ratios (E:T) of 0.5:1. FIG. 16B shows the amount of IFNγ in supernatant collected from the cytotoxicity assay of FIG. 16A by using HTRF.

FIG. 17A shows the result of cytotoxicity assay, on day 6 post transfection, where anti-BCMA system: anti-BCMA2-anti-BCMA3 epsilon-TCR/anti-BCMA2-anti-BCMA3 gamma-TCR (tandem BCMA2&3 eTCR/gTCR), anti-BCMA2-anti-BCMA3 gamma-TCR/anti-BCMA2-anti-BCMA3 4-1BB-CD3zeta CAR (tandem BCMA2&3 gTCR/BBzCAR), as well as control untransduced cells are co-cultured with RPMI-8226 cells (BCMA+) at effector-to-target cell ratios (E:T) of 0.33:1. FIG. 17B shows the amount of IFNγ in supernatant collected from the cytotoxicity assay of FIG. 17A by using HTRF.

FIG. 18 shows the in vivo anti-tumor efficacy of tri-specific BCMA CAR-T cells, tri-specific BCMA TCR-T cells and tri-specific BCMA CAR-TCR-T cells evaluated in a NCG mouse model (NOD_Prkdcem26Cd52/NjuCrl) having a multiple myeloma tumor xenograft.

FIG. 19 shows the in vivo anti-tumor efficacy of anti-MSLN/FSHR double CAR-T (MSLN CAR+FSHR CAR), anti-MSLN/FSHR double eTCR-T (MSLN eTCR+FSHR eTCR) and anti-MSLN CAR/FSHR eTCR-T (MSLN CAR+FSHR eTCR) assessed in an OVCAR-8 xenograft model. 10×10⁶ OVCAR-8 cells were implanted subcutaneously on day 0 in NOD scid gamma (NSG) mice. Once tumors were 150-200 mm³, the mice were randomized into treatment groups. 0.33×10⁶ CAR positive T cells in a 200 μl dose were administered intravenously. The mice and tumors of the mice were monitored for about 60 days after tumor cell implantation.

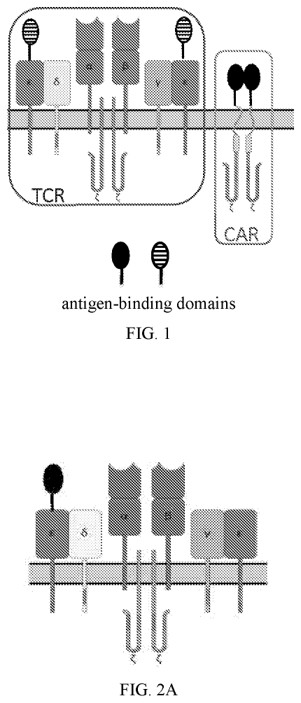

### DETAILED DESCRIPTION

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used in the specification and claims, the singular forms “a,” “an,” and “the” include plural references unless the context clearly dictates otherwise. For example, the term “an antigen binding domain” includes a plurality of antigen binding domains.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

As used herein, the term “T-cell” or “T lymphocyte” refers to a type of lymphocyte that plays a central role in cell-mediated immunity. T cells can be distinguished from other lymphocytes, such as B cells and natural killer cells, by the presence of a T-cell receptor on the cell surface.

As used herein, the term “T-cell receptor” or “TCR” refers to a molecule on the surface of a T cell or T lymphocyte that is responsible for recognizing an antigen. TCR is a heterodimer which is composed of two different protein chains. In some embodiments, the TCR of the present disclosure consists of an alpha (α) chain and a beta (β) chain and is referred as αβ TCR. αβ TCR recognizes antigenic peptides degraded from protein bound to major histocompatibility complex molecules (MHC) at the cell surface. In some embodiments, the TCR of the present disclosure consists of a gamma (γ) and a delta (δ) chain and is referred as γδ TCR. γδ TCR recognizes peptide and non-peptide antigens in a MHC-independent manner. γδ T cells have shown to play a prominent role in recognizing lipid antigens. In particular, the γ chain of TCR includes but is not limited to Vγ2, Vγ3, Vγ4, Vγ5, Vγ8, Vγ9, Vγ10, a functional variant thereof, and a combination thereof; and the δ chain of TCR includes but is not limited to δ1, δ2, δ3, a functional variant thereof, and a combination thereof. In some embodiments, the γδ TCR may be Vγ2/Vδ1TCR, Vγ2/Vδ2 TCR, Vγ2/Vδ3 TCR, Vγ3/Vδ1 TCR, Vγ3/Vδ2 TCR, Vγ3/Vδ3 TCR, Vγ4/Vδ1 TCR, Vγ4/Vδ2 TCR, Vγ4/Vδ3 TCR, Vγ5/Vδ1 TCR, Vγ5/Vδ2 TCR, Vγ5/Vδ3 TCR, Vγ8/Vδ1 TCR, Vγ8/Vδ2 TCR, Vγ8/Vδ3 TCR, Vγ9/Vδ1 TCR, Vγ9/Vδ2 TCR, Vγ9/Vδ3 TCR, Vγ10/Vδ1 TCR, Vγ10/Vδ2 TCR, and/or Vγ10/Vδ3 TCR. In some examples, the γδ TCR may be Vγ9/Vδ2 TCR, Vγ10/Vδ2 TCR, and/or Vγ2/Vδ2 TCR.

As used herein, the term “alpha beta T cell”, “αβ T cell” and “AB T cell” can be used interchangeably and refer to a T cell (T lymphocyte) that comprises αβ TCR, or a variant or fragment thereof, whereas the terms “gamma delta T cell”, “γδ T cell” and “GD T cell” can be used interchangeably and refer to a T cell (T lymphocyte) that comprises γδ TCR, or a variant or fragment thereof, for example, Vγ962 T cells, Vδ1 T cells, Vδ3 T cells or Vδ5 T cells. In some embodiments, the γδ T cells may be Vγ2/Vδ1T cells, Vγ2/Vδ2 T cells, Vγ2/Vδ3 T cells, Vγ3/Vδ1 T cells, Vγ3/Vδ2 T cells, Vγ3/Vδ3 T cells, Vγ4/Vδ1 T cells, Vγ4/Vδ2 T cells, Vγ4/Vδ3 T cells, Vγ5/Vδ1 T cells, Vγ5/Vδ2 T cells, Vγ5/Vδ3 T cells, Vγ8/Vδ1 T cells, Vγ8/Vδ2 T cells, Vγ8/Vδ3 T cells, Vγ9/Vδ1 T cells, Vγ9/Vδ2 T cells, Vγ9/Vδ3 T cells, Vγ10/Vδ1 T cells, Vγ10/Vδ2 T cells, and/or Vγ10/Vδ3 T cells. In some examples, the γδ T cell may be Vγ9/Vδ2 T cell, Vγ10/Vδ2 T cell, and/or Vγ2/Vδ2 T cell.

The term “activation” and its grammatical equivalents as used herein can refer to a process whereby a cell transitions from a resting state to an active state. This process can comprise a response to an antigen, migration, and/or a phenotypic or genetic change to a functionally active state. For example, the term “activation” can refer to the stepwise process of T cell activation. In some cases, a T cell can require at least two signals to become fully activated. The first signal can occur after engagement of a TCR by the antigen-MHC complex, and the second signal can occur by engagement of co-stimulatory molecules. In some cases, anti-CD3 can mimic the first signal and anti-CD28 can mimic the second signal in vitro.

The term “antigen,” as used herein, refers to a molecule or a fragment thereof capable of being bound by a selective binding agent. As an example, an antigen can be a ligand that can be bound by a selective binding agent such as a receptor. In some cases, the receptor may function as the antigen and the ligand may function as the selective binding agent. As another example, an antigen can be an antigenic molecule that can be bound by a selective binding agent such as an immunological protein (e.g., an antibody). In some cases, the immunological protein may serve as the antigen and the antigenic molecule may serve as the selective binding agent. An antigen can also refer to a molecule or fragment thereof capable of being used in an animal to produce antibodies capable of binding to that antigen.

The term “epitope” and its grammatical equivalents, as used herein, can refer to a part of an antigen that can be recognized by an antigen binding domain. Antigen binding domains can comprise, for example, proteins (e.g., antibodies, antibody fragments) present on a surface, for example a cell surface (e.g., B cells, T cells, CAR-T cells, or engineered cells). For example, an epitope can be a cancer epitope that is recognized by a TCR. Multiple epitopes within an antigen can also be recognized. The epitope can also be mutated.

The term “antigen binding molecule” as used herein refers to a molecule that specifically binds to an antigen or epitope. Examples of the antigen binding molecule include but are not limited to antibody and derivatives thereof, e.g. a fragment thereof. By “specifically binding,” it means that the binding is selective for the antigen or epitope, and can be discriminated from unwanted or non-specific interactions.

The term “binding affinity” as used herein refers to strength of the binding interaction between members of a binding pair, for example, an antigen binding molecule and its antigen, or a receptor and its ligand.

The binding affinity of a subject antibody for its partner may be characterized by kₒₙ, k_{off} or K_{D}. The term “kₒₙ”, as used herein, is intended to refer to the rate constant for association of an antibody to an antigen. The term “k_{off}”, as used herein, is intended to refer to the rate constant for dissociation of an antibody from the antibody/antigen complex. The term “K_{D}”, as used herein, is intended to refer to the equilibrium dissociation constant of an antibody-antigen interaction. For purposes of the present disclosure, K_{D} is defined as the ratio of the two kinetic rate constants kₒₙ/k_{off}. The smaller the equilibrium dissociation constant the tighter the subject antibody and its partner bind to each other.

The term “antibody,” as used herein, refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)₂, variable fragment (Fv), single chain variable fragment (scFv), minibodies, diabodies, and single-domain antibodies (“sdAb” or “nanobodies” or “camelids” or V_{H}H). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The term “antigen binding domain,” as used herein, refers to a protein or fragment thereof capable of binding an antigen or an epitope. As an example, an antigen binding domain can be a cellular receptor. As an example, an antigen binding domain can be an engineered cellular receptor. As an example, an antigen binding domain can be a soluble receptor. In some cases, an antigen binding domain can be the ligand which is bound by the cellular receptor, the engineered cellular receptor, and/or the soluble receptor.

The term “autologous” and its grammatical equivalents, as used herein, can refer to origination from the same being. For example, an autologous sample (e.g., cells) can refer to a sample which is removed, processed, and then given back to the same subject (e.g., patient) at a later time. Autologous, with respect to a process, can be distinguished from an allogenic process in which the donor of a sample (e.g., cells) and the recipient of the sample are not the same subject.

The terms “cancer neo-antigen,” “neo-antigen,” and “neo-epitope” and their grammatical equivalents, as used herein, can refer to antigens that are not encoded in a normal, non-mutated host genome. A “neo-antigen” can, in some instances, represent either oncogenic viral proteins or abnormal proteins that arise as a consequence of somatic mutations. For example, a neo-antigen can arise by the disruption of cellular mechanisms through the activity of viral proteins. As another example, a neo-antigen can arise from exposure to a carcinogenic compound, which in some cases can lead to a somatic mutation. This somatic mutation can lead to the formation of a tumor/cancer.

The term “cytotoxicity,” as used herein, refers to an unintended or undesirable alteration in the normal state of a cell. The normal state of a cell may refer to a state that is manifested or exists prior to the cell's exposure to a cytotoxic composition, agent and/or condition. A cell that is in a normal state can be in homeostasis. An unintended or undesirable alteration in the normal state of a cell can be manifested in the form of, for example, cell death (e.g., programmed cell death), a decrease in replicative potential, a decrease in cellular integrity such as membrane integrity, a decrease in metabolic activity, a decrease in developmental capability, or any of the cytotoxic effects disclosed herein.

The phrases “reducing cytotoxicity” and “reduce cytotoxicity,” as used herein, refer to a reduction in degree or frequency of unintended or undesirable alterations in the normal state of a cell upon exposure to a cytotoxic composition, agent and/or condition. The phrase can refer to reducing the degree of cytotoxicity in an individual cell that is exposed to a cytotoxic composition, agent and/or condition, or to reducing the number of cells of a population that exhibit cytotoxicity when the population of cells is exposed to a cytotoxic composition, agent and/or condition.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell.

The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including, but not limited to, a therapeutic benefit and/or a prophylactic benefit. For example, a treatment can comprise administering a system or cell population disclosed herein. A therapeutic benefit can refer to any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

A “therapeutic effect” may occur if there is a change in the condition being treated. The change may be positive or negative. For example, a ‘positive effect’ may correspond to an increase in the number of activated T-cells in a subject. In another example, a ‘negative effect’ may correspond to a decrease in the amount or size of a tumor in a subject. A “change” in the condition being treated, may refer to at least 1%, 2%, 3%, 4%, 5%, 6%, 7%, 8%, 9%, 10%, 25%, 50%, 75%, or 100% change in the condition. The change can be based on improvements in the severity of the treated condition in an individual, or on a difference in the frequency of improved conditions in populations of individuals with and without the administration of a therapy. Similarly, a method of the present disclosure may comprise administering to a subject an amount of cells that is “therapeutically effective”. The term “therapeutically effective” should be understood to have a definition corresponding to ‘having a therapeutic effect’.

The term “effective amount” or “therapeutically effective amount” refers to the quantity of a composition, for example a composition comprising immune cells such as lymphocytes (e.g., T lymphocytes and/or NK cells), that is sufficient to result in a desired activity upon administration to a subject in need thereof. The term “therapeutically effective” can refer to a quantity of a composition that is sufficient to delay the manifestation, arrest the progression, relieve or alleviate at least one symptom of a disorder treated by the methods of the present disclosure.

The term “TIL” or tumor infiltrating lymphocyte and its grammatical equivalents, as used herein, can refer to a cell isolated from a tumor. A TIL can be any cell found within a tumor. For example, a TIL can be a cell that has migrated to a tumor. A TIL can be a cell that has infiltrated a tumor. A TIL can be a T cell, B cell, monocyte, natural killer (NK) cell, or any combination thereof. A TIL can be a mixed population of cells. A population of TILs can comprise cells of different phenotypes, cells of different degrees of differentiation, cells of different lineages, or any combination thereof.

The term “B-cell maturation antigen (BCMA or BCM)”, also known as tumor necrosis factor receptor superfamily member 17 (TNFRSF17), refers to a protein encoded by the TNFRSF17 gene in human. BCMA is preferentially expressed in mature B lymphocytes, and has been proved to have important roles for B cell development and autoimmune response. BCMA is also regarded as a tumor-associated antigen, and the abnormal expression of BCMA has been also linked to a number of cancers, as well as autoimmune disorders and infectious diseases.

In one aspect, provided herein is an antigen binding molecule having a formula of A-X-B-Y-C-Z-D. In some embodiments, the present disclosure provides an antigen binding molecule having the formula A-X-B-Y-C-Z-D, and said A comprises a sequence having at least 80%, 85%, 90%, 95%, 96%, 97%, 98%, 99% or 100% identity to any one selected from the group consisting of SEQ ID NOs: 47-56. In some embodiments, the present disclosure provides an antigen binding molecule having the formula A-X-B-Y-C-Z-D, and said B comprises a sequence having at least 80%, 85%, 90%, 95%, 96%, 97%, 98%, 99% or 100% identity to any one selected from the group consisting of SEQ ID NOs: 57-66. In some embodiments, the present disclosure provides an antigen binding molecule having the formula A-X-B-Y-C-Z-D, and said C comprises a sequence having at least 80%, 85%, 90%, 95%, 96%, 97%, 98%, 99% or 100% identity to any one selected from the group consisting of SEQ ID NOs: 67-76. In some embodiments, the present disclosure provides an antigen binding molecule having the formula A-X-B-Y-C-Z-D, and said D comprises a sequence having at least 80%, 85%, 90%, 95%, 96%, 97%, 98%, 99% or 10000 identity to any one selected from the group consisting of SEQ TD NOs 77-86. In some embodiments, the present disclosure provides an antigen binding molecule having the formula A-X-B-Y-C-Z-D, and said X comprises a sequence having at least 80%, 85%, 90%, 95%, 96%, 97%, 98%, 99% or 100% identity to any one selected from the group consisting of SEQ TD NOs: 87-96. In some embodiments, the present disclosure provides an antigen binding molecule having the formula A-X-B-Y-C-Z-D, and said Y comprises a sequence having at least 80%, 85%, 90%, 95%, 96%, 97%, 98%, 99% or 100% identity to any one selected from the group consisting of SEQ TD NOs:97-106. In some embodiments, the present disclosure provides an antigen binding molecule having the formula A-X-B-Y-C-Z-D, and said Z comprises a sequence having at least 80%, 85%, 90%, 95%, 96%, 97%, 98%, 99% or 100% identity to any one selected from the group consisting of SEQ TD NOs:107-116.

TABLE 1
Sequences of A of the antigen binding molecule

| SEQ ID NO | A |
|---|---|
| 47 | QVQLVESGGGSVQAGGSLRLSCKAS |
| 48 | QVQLEESGGGSVQAGGSLRLSCAYT |
| 49 | QMQLVESGGGSVQAGGSLRLSCTAS |
| 50 | QVHLMESGGGSVQSGGSLRLSCAAS |
| 51 | QVQLVESGGGSVQAGGSLRLSCAAS |
| 52 | QVQLVESGGGSVQAGGSLRLSCKSS |
| 53 | QVQLAESGGGLVQPGGSLRLSCAGS |
| 54 | QVQLVESGGGVVQPGGSLRLSCAAS |
| 55 | QVHLVESGGGSVQAGGSLRLSCKSS |
| 56 | QVHLVESGGGSVQAGGSLRLSCKAS |

TABLE 2
Sequences of B of the antigen binding molecule

| SEQ ID NO | B |
|---|---|
| 57 | WFRQTPGKEREGVA |
| 58 | WFREAPGKARTSVA |
| 59 | WYRQAPGNECELV |
| 60 | WFRQAPGKEREGVA |
| 61 | WFRQAPGKEREDVA |
| 62 | WFRQTPGKGREGVA |
| 63 | WVRQAPGKGLERVS |
| 64 | WGRQAPGQRLEWVS |
| 65 | WFRQTPGKEREGVA |
| 66 | WFRQTPGKEREGVA |

TABLE 3
Sequences of C of the antigen binding molecule

| SEQ ID NO | C |
|---|---|
| 67 | RFTISRDNAKNTMYLQMNSLEPEDTAMYYCAA |
| 68 | RFTISKDNAKNTLYLQMNSLKPEDSAMYRCAA |
| 69 | RFTISQDNAKNTMYLQMNSLKPEDTAVYSCAA |
| 70 | RFTISQDNAKNTLYLQMNSLKPEDTAMYYCGA |
| 71 | RFTISQDTAQNTLYLQMNSLKPEDTAMYYCAA |
| 72 | RFTISRDNAKNTMYLQMNSLKPEDTAMYYCAA |
| 73 | RFTASRDKAKNTLYLQMNSLKTEDTAVYYCAA |
| 74 | RFTISRDNAKNTLYLQLNNLKSEDTAVYYCSE |
| 75 | RFTISRDNAKNTMYLQMSGLRPEDTALYYCAA |
| 76 | RFTISRDNAKNTMYLQMNSLKPEDTAMYYCAA |

TABLE 4
Sequences of D of the antigen binding molecule

| SEQ ID NO | D |
|---|---|
| 77 | WGQGTQVTVSS |
| 78 | WGQGTQVTVSS |
| 79 | WGQGTQVTVSS |
| 80 | WGQGTQVTVSS |
| 81 | WGQGTQVTVSS |
| 82 | WGQGTQVTVSS |
| 83 | WGQGTQVTVSS |
| 84 | WGQGTQVTVSS |
| 85 | WGQGTQVTVSS |
| 86 | WGQGTQVTVSS |

TABLE 5
Sequences of X of the antigen binding molecule

| SEQ ID NO | X |
|---|---|
| 87 | GAIYDTNCMA |
| 88 | YSTYSNYYMG |
| 89 | GYTFDDSAMG |
| 90 | GYTYSSYCMA |
| 91 | GGTRSWNYMA |
| 92 | GAPYSSNCMA |
| 93 | GFTFSSYDMN |
| 94 | GFAFSNYAMT |
| 95 | GATYSSNCMA |
| 96 | GAIYDTNCMA |

TABLE 6
Sequences of Y of the antigen binding molecule

| SEQ ID NO | Y |
|---|---|
| 97 | TIDLGNPITYYADSVKG |
| 98 | IISSDTTITYKDAVKG |
| 99 | SISSDGSTYYSDSVKG |
| 100 | AIASDGSTYYTDSVKG |
| 101 | IIDNVGSTRYADSVKG |
| 102 | TIDLASHDTYYADSVKG |
| 103 | TTFNGDDGTNYADSVLG |
| 104 | TIDSGGGSTTYSDSVKG |
| 105 | TIDLASHGTYYADSVKG |
| 106 | TIDLGNPITYYADSVKG |

TABLE 7
Sequences of Z of the antigen binding molecule

| SEQ ID NO | Z |
|---|---|
| 107 | TSWWPCTTFNAGYAN |
| 108 | WTSDWSVAY |
| 109 | SSGEDGGSWSTPCHFFGY |
| 110 | DPVGCSWPDY |
| 111 | RVSWCEDPPCGFDY |
| 112 | TSWWPCTTFNGGYAN |
| 113 | AVPGVDWYDTTRYKY |
| 114 | NVDCNGDYCYRANY |
| 115 | TSWWPCTTFNGGYAS |
| 116 | TSWWPCPANNVGYAN |

In some embodiments, the present disclosure provides an antigen binding molecule having the formula A-X-B-Y-C-Z-D, wherein A comprises a sequence having at least 80% or 90% identity to any one selected from the group consisting of SEQ ID NOs: 47-56, B comprises a sequence having at least 80% or 90% identity to any one selected from the group consisting of SEQ ID NOs: 57-66, C comprises a sequence having at least 80% or 90% identity to any one selected from the group consisting of SEQ ID NOs: 67-76, D comprises a sequence having at least 80% or 90% identity to any one selected from the group consisting of SEQ ID NOs: 77-86, X comprises a sequence having at least 80% or 90% identity to any one selected from the group consisting of SEQ ID NOs: 87-96, Y comprises a sequence having at least 80% or 90% identity to any one selected from the group consisting of SEQ ID NOs: 97-106, and Z comprises a sequence having at least 80% or 90% identity to any one selected from the group consisting of SEQ ID NOs: 107-116.

In some embodiments, the antigen binding molecule exhibits a binding affinity (K_{D}) for human BCMA. In some embodiments, the K_{D} is less than 100 nm, 90 nm, 80 nm, 70 nm, 60 nm, 50 nm, 40 nm, 30 nm, 20 nm, 10 nm, or 1 nm or less as determined by surface plasmon resonance at 37° C.

In some embodiments, the antigen binding molecule comprises a sequence having at least 80%, 85%, 90%, 95%, 96%, 97%, 98%, 99% or 100% identity to any one selected from the group consisting of SEQ ID NOs: 14-23. In some embodiments, the antigen binding molecule comprises a sequence selected from the group consisting of SEQ ID NOs: 14-23.

In one aspect, the present disclosure provides a modified T cell receptor (TCR) complex comprising an antigen binding domain which exhibits specific binding to an epitope, wherein the antigen binding domain is linked to: (i) at least one TCR chain selected from an alpha chain, a beta chain, a gamma chain and a delta chain of a TCR; (ii) an epsilon chain, a delta chain, and/or a gamma chain of a cluster of differentiation 3 (CD3); or (iii) a CD3 zeta chain.

In some embodiments, the antigen binding domain can comprise one member of an interacting pair. For example, the antigen binding domain may be one member, or a fragment thereof, of an interacting pair comprising a receptor and a ligand. Either the receptor or ligand, or fragments thereof, may be referred to as the antigen binding domain. The other member which is not referred to as the antigen binding domain can comprise the epitope to which the antigen binding domain specifically binds.

Non-limiting examples of the antigen binding domain of the TCR complex include, but are not limited to, a monoclonal antibody, a polyclonal antibody, a recombinant antibody, a human antibody, a humanized antibody, or a functional derivative, variant or fragment thereof, including, but not limited to, a Fab, a Fab′, a F(ab′)₂, an Fv, a single-chain Fv (scFv), minibody, a diabody, and a single-domain antibody such as a heavy chain variable domain (VH), a light chain variable domain (VL) and a variable domain (V_{H}H) of camelid derived Nanobody. In some embodiments, the antigen binding domain of the TCR complex comprises at least one of a Fab, a Fab′, a F(ab′)₂, an Fv, and a scFv. In some embodiments, the antigen binding domain of the TCR complex comprises an antibody mimetic. Antibody mimetics refer to molecules which can bind a target molecule with an affinity comparable to an antibody, and include single-chain binding molecules, cytochrome b562-based binding molecules, fibronectin or fibronectin-like protein scaffolds (e.g., adnectins), lipocalin scaffolds, calixarene scaffolds, A-domains and other scaffolds. In some embodiments, an antigen binding domain comprises a transmembrane receptor, or any derivative, variant, or fragment thereof. For example, an antigen binding domain can comprise at least a ligand binding domain of a transmembrane receptor.

In some embodiments, provided herein is a modified T cell receptor (TCR) complex comprising one or more antigen binding domains, wherein said one or more antigen binding domains are linked to: (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain; and wherein at least one or two of the one or more antigen binding domains comprises an antigen binding molecule described herein.

In some embodiments, at least one antigen binding domain of the modified TCR complex comprises a sequence having at least 80%, 85%, 90%, 95%, 96%, 97%, 98%, 99% or 100% identity to any one selected from the group consisting of SEQ ID NOs: 3-23, and 38-46.

In some embodiments, the antigen binding domain of the TCR complex comprises a single-domain antibody. In some embodiments, said single-domain antibody is an anti-BCMA sdAb disclosed herein. In some embodiments, said anti-BCMA sdAb comprises a sequence having at least 80%, 85%, 90%, 95%, 96%, 97%, 98%, 99% or 100% identity to any one selected from SEQ ID NOs: 3-23. In some embodiments, said anti-BCMA sdAbs comprises a sequence of any one selected from SEQ ID NOs: 3-23.

The antigen binding domain of the modified TCR complex can be linked to any member of the TCR complex. In some embodiments, the antigen binding domain can be linked to at least one of a TCR chain, a CD3 chain, or CD3 zeta chain. In some embodiments, the antigen binding domain can be linked to transmembrane receptor of a TCR, for example, TCR-epsilon, TCR-delta, TCR-gamma, TCR-alpha, or TCR-beta. In some embodiments, the antigen binding domain can be linked to a CD3 chain, for example, CD3-epsilon, CD3-delta, or CD3-gamma. In some embodiments, the antigen binding domain can be linked to CD3 zeta chain.

The modified T cell receptor (TCR) complex of the present disclosure can comprise a second antigen binding domain which exhibits binding to a second epitope. The second antigen binding domain can comprise any protein or molecule that can bind to an epitope. Non-limiting examples of the second antigen binding domain of the TCR complex include, but are not limited to, a monoclonal antibody, a polyclonal antibody, a recombinant antibody, a human antibody, a humanized antibody, or a functional derivative, variant or fragment thereof, including, but not limited to, a Fab, a Fab′, a F(ab′)₂, an Fv, a single-chain Fv (scFv), minibody, a diabody, and a single-domain antibody such as a heavy chain variable domain (VH), a light chain variable domain (VL) and a variable domain (V_{H}H) of camelid derived Nanobody. In some embodiments, the second antigen binding domain of the TCR complex comprises at least one of a Fab, a Fab′, a F(ab′)₂, an Fv, and a scFv. In some embodiments, the second antigen binding domain of the TCR complex comprises an antibody mimetic. Antibody mimetics refer to molecules which can bind a target molecule with an affinity comparable to an antibody, and include single-chain binding molecules, cytochrome b562-based binding molecules, fibronectin or fibronectin-like protein scaffolds (e.g., adnectins), lipocalin scaffolds, calixarene scaffolds, A-domains and other scaffolds. In some embodiments, an antigen binding domain comprises a transmembrane receptor, or any derivative, variant, or fragment thereof. For example, an antigen binding domain can comprise at least a ligand binding domain of a transmembrane receptor.

In some embodiments, the second antigen binding domain of the TCR complex comprises an antigen binding molecule disclosed herein. In some embodiments, the second antigen binding domain of the TCR complex comprises a single-domain antibody. In some embodiments, said single-domain antibody is an anti-BCMA sdAb. In some embodiments, said anti-BCMA sdAb comprises a sequence having at least 80%, 85%, 90%, 95%, 96%, 97%, 98%, 99% or 100% identity to any one selected from SEQ ID NOs: 3-23. In some embodiments, said anti-BCMA sdAb comprises a sequence of any one selected from SEQ ID NOs: 3-23. In some embodiments, the second antigen binding domain of the TCR complex comprises a sequence having at least 80% or 90% identity to any one selected from the group consisting of SEQ ID NOs: 38-46.

The second antigen binding domain can be linked to any member of the TCR complex. In some embodiments, the second antigen binding domain can be linked to at least one of a TCR chain, a cluster of differentiation 3 (CD3) chain, or CD3 zeta chain. The second antigen binding domain can be linked to transmembrane receptor of a TCR, for example, TCR-epsilon, TCR-delta, TCR-gamma, TCR-alpha, or TCR-beta. The second antigen binding domain can be linked to a CD3 chain, for example, CD3-epsilon, CD3-delta, or CD3-gamma. The second antigen binding domain can be linked to CD3 zeta chain.

In some embodiments, the two or more antigen binding domains are linked to separate chains of the TCR complex. In some embodiment, the two or more antigen binding domains are linked to one chain of the TCR complex. Any number of antigen binding domains can be used in the modified TCR complex of the present disclosure, and the number of antigen binding domains is not limited to one, two or three.

In some embodiments, the two or more antigen binding domains can be the same antigen binding domain. For example, the two or more antigen binding domains may be identical molecules capable of binding to the same ligand. In some embodiments, the two or more antigen binding domains can be different antigen binding domains. For example, the two or more antigen binding domains may be different molecules capable of binding to the same ligand or different ligands.

In some embodiment, the two or more antigen binding domains are linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain. In some embodiment, the two or more antigen binding domains are linked in tandem to at least one of an epsilon chain, a delta chain, and/or a gamma chain of a cluster of differentiation 3 (CD3). In some embodiment, the two or more antigen binding domains are linked in tandem to separate chains of the TCR complex. In some embodiment, the two or more antigen binding domains are linked in tandem to one chain of the modified TCR complex. In some embodiment, the two or more antigen binding domains are linked in tandem to two or more chains of the modified TCR complex.

In some embodiments, the modified TCR complex of the present disclosure comprises two or more sdAbs linked to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain. In some embodiments, the modified TCR complex of the present disclosure comprises two or more sdAbs linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain. In some embodiments, the modified TCR complex of the present disclosure comprises two or more sdAbs linked in tandem to one chain of the modified TCR complex. In some embodiments, the modified TCR complex of the present disclosure comprises two or more sdAbs linked in tandem to two or more chains of the modified TCR complex.

In some embodiments, the modified TCR complex of the present disclosure comprises two or more anti-BCMA sdAbs linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain. In some embodiments, the two or more anti-BCMA sdAbs have the same sequence. In some embodiments, the two or more anti-BCMA sdAbs have different sequences. In some embodiments, the modified TCR complex of the present disclosure comprises two or more anti-BCMA sdAbs linked in tandem to one chain of the TCR complex. In some embodiments, the modified TCR complex of the present disclosure comprises two or more anti-BCMA sdAbs linked in tandem to two or more chains of the TCR complex.

In some embodiments, the modified TCR complex of the present disclosure comprises two or more anti-BCMA antigen binding molecules disclosed herein linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain. In some embodiments, the modified TCR complex of the present disclosure comprises two or more anti-BCMA sdAbs linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain, and the anti-BCMA sdAbs comprises a sequence having at least 80%, 85%, 90%, 95%, 96%, 97%, 98%, 99% or 100% identity to any one selected from SEQ ID NOs: 3-23. In some embodiments, the two or more anti-BCMA sdAbs have the same sequence, and the sequence has at least 80%, 85%, 90%, 95%, 96%, 97%, 98%, 99% or 100% identity to any one selected from SEQ ID NOs: 3-23. In some embodiments, the two or more anti-BCMA sdAbs have different sequences, and the sequences have at least 80%, 85%, 90%, 95%, 96%, 97%, 98%, 99% or 100% identity to any one selected from SEQ ID NOs: 3-23. In some embodiments, the modified TCR complex of the present disclosure comprises two or more anti-BCMA sdAbs linked in tandem to one chain of the TCR complex, and the anti-BCMA sdAbs comprises a sequence having at least 80%, 85%, 90%, 95%, 96%, 97%, 98%, 99% or 100% identity to any one selected from SEQ ID NOs: 3-23. In some embodiments, the modified TCR complex of the present disclosure comprises two or more anti-BCMA sdAbs linked in tandem to two or more chains of the TCR complex, and the anti-BCMA sdAbs comprises a sequence having at least 80%, 85%, 90%, 95%, 96%, 97%, 98%, 99% or 100% identity to any one selected from SEQ ID NOs: 3-23.

In some embodiments, the two or more antigen binding domains of the modified TCR complex can bind to epitopes present on different antigens. In some embodiments, the two or more antigen binding domains of the modified TCR complex can bind epitopes present on a common antigen. In some embodiments, the two or more antigen binding domains exhibit specific binding to two or more epitopes. In some embodiments, the two or more antigen binding domains exhibit specific binding to the same epitope.

Accordingly, also provided herein is a modified T cell receptor (TCR) complex comprising two or more antigen binding domains exhibiting specific binding to two or more epitopes, wherein said two or more antigen binding domains are linked to: (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain. In some embodiments, at least one or two of the two or more antigen binding domains are selected from the antigen binding domains or the antigen binding molecules disclosed herein.

In some embodiments, the epitope that the antigen binding domain of the modified TCR complex binds to may be present on one or more cell surface antigens. The one or more cell surface antigens can be tyrosine kinase receptors, serine kinase receptors, histidine kinase receptor, G-protein coupled receptors (GPCR), and the like.

In some embodiments, the epitope that the antigen binding domain of the modified TCR complex binds to may be present on an immune checkpoint receptor or immune checkpoint receptor ligand. In some embodiments, the immune checkpoint receptor or immune checkpoint receptor ligand can be PD-1, PD-L1, PD-L2, CTLA-4, TIM-3, LAG3, TIGIT, BLTA, CD47 or CD40.

In some embodiments, the epitope that the antigen binding domain of the modified TCR complex binds to may be present on a cytokine or a cytokine receptor. A cytokine receptor can be, for example, CCR2b, CXCR2 (CXCL1 receptor), CCR4 (CCL17 receptor), Gro-a, IL-2, IL-7, IL-15, IL-21, IL-12, Heparanase, CD137L, LEM, Bcl-2, CCL17, CCL19 or CCL2.

In some embodiments, the epitope that the antigen binding domain of the modified TCR complex binds to may be present on a tumor-associated antigen. The epitope may be, for instance a tumor epitope. A tumor-associated antigen can be selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFαRII, TGFβRII, TP1, TRAG-3, TRG, TRP-1, TRP-2, TRP-2/INT2, TRP-2-6b, Tyrosinase, VEGF-R2, WT1, α-folate receptor, and κ-light chain. In some embodiments, the epitope that the two or more antigen binding domains of the modified TCR complex binds to can be EGFR, EGFRvIII, GPC3, GPC-2, DLL3, CD19, CD20, CD22, CD123, CLL-1, CD30, CD33, HER2, MSLN, PSMA, CEA, GD2, IL13Rα2, CAIX, L1-CAM, CA125, CD133, FAP, CTAG1B, MUC1, FR-α, CD70, CD171, ROR1, and any combination thereof.

In some embodiments, at least one of the antigen binding domains of the modified TCR complex binds to an epitope present on BCMA. In some embodiments, two or more antigen binding domains of the modified TCR complex bind to an epitope present on BCMA. In some embodiments, two or more antigen binding domains of the modified TCR complex bind to the same epitope of BCMA. In some embodiments, two or more antigen binding domains of the modified TCR complex bind to different epitopes of BCMA.

In some embodiments, two or more antigen binding domains of the modified TCR complex are linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain, and at least one of the binding domains can bind to BCMA. In some embodiments, two or more antigen binding domains of the modified TCR complex are linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain, and the two or more of the antigen binding domains can bind to BCMA. In some embodiments, two or more antigen binding domains of the modified TCR complex are linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain, and the two or more antigen binding domains can bind to the same epitope of BCMA. In some embodiments, two or more antigen binding domains of the TCR complex are linked in tandem to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain, and the two or more antigen binding domains can bind to different epitopes of BCMA.

In some embodiments, the epitope that the antigen binding domain of the modified TCR complex binds to may be present on a neoantigen. For example, the epitope may be a neoepitope.

Neoantigens and neoepitopes generally refer to tumor-specific mutations that in some cases trigger an antitumor T cell response. For example, these endogenous mutations can be identified using a whole-exomic-sequencing approach. Tran E, et al., “Cancer immunotherapy based on mutation-specific CD4+ T cells in a patient with epithelial cancer,” Science 344: 641-644 (2014). An antigen binding domain, for example, that of a subject CAR or a modified TCR complex can exhibit specific binding to a tumor-specific neo-antigen. Neoantigens bound by antigen binding domains the modified TCR complex can be expressed on a target cell, and for example, are neoantigens and neoeptiopes encoded by mutations in any endogenous gene. In some cases, the two or more antigen binding domains bind a neoantigen or neoepitope encoded by a mutated gene. The gene can be selected from the group consisting of: ABL1, ACOl 1997, ACVR2A, AFP, AKT1, ALK, ALPPL2, ANAPC1, APC, ARID1A, AR, AR-v7, ASCL2, β2M, BRAF, BTK, C15ORF40, CDH1, CLDN6, CNOT1, CT45A5, CTAG1B, DCT, DKK4, EEF1B2, EEF1DP3, EGFR, EIF2B3, env, EPHB2, ERBB3, ESR1, ESRP1, FAM11 IB, FGFR3, FRG1B, GAGE1, GAGE 10, GATA3, GBP3, HER2, IDH1, JAK1, KIT, KRAS, LMAN1, MABEB 16, MAGEA1, MAGEA10, MAGEA4, MAGEA8, MAGEB 17, MAGEB4, MAGEC1, MEK, MLANA, MLL2, MMP13, MSH3, MSH6, MYC, NDUFC2, NRAS, NY-ESO, PAGE2, PAGE5, PDGFRa, PIK3CA, PMEL, pol protein, POLE, PTEN, RAC1, RBM27, RNF43, RPL22, RUNX1, SEC31A, SEC63, SF3B 1, SLC35F5, SLC45A2, SMAP1, SMAP1, SPOP, TFAM, TGFBR2, THAP5, TP53, TTK, TYR, UBR5, VHL, and XPOT.

In some embodiments, the epitope that the antigen binding domain of the modified TCR complex binds to may be present on a stroma. Stroma generally refers to tissue which, among other things, provides connective and functional support of a biological cell, tissue, or organ. A stroma can be that of the tumor microenvironment. The epitope may be present on a stromal antigen. Such an antigen can be on the stroma of the tumor microenvironment. Neoantigens and neoepitopes, for example, can be present on tumor endothelial cells, tumor vasculature, tumor fibroblasts, tumor pericytes, tumor stroma, and/or tumor mesenchymal cells. Example antigens include, but are not limited to, CD34, MCSP, FAP, CD31, PCNA, CD117, CD40, MMP4, and Tenascin.

In some embodiments, epitope can be present on an antigen presented by a major histocompatibility complex (MHC). An MHC can be human leukocyte antigen (HLA) class I or class II. An HLA can be HLA-A, HLA-B, HLA-C, HLA-HLA-E, HLA-F, HLA-G, HLA-DP, HLA-DQ, HLA-DR, HLA-DM, or HLA-DO. In some embodiments, the epitope can be present on HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, or HLA-A*24, HLA-B*27, HLA-B*35, HLA-B*48, HLA-B*55, and the like.

In some embodiments, the epitope can be soluble (e.g., not bound to a cell). In some cases, the antigen can be soluble, e.g., a soluble antigen. The epitope may be present on a universal antigen. In some cases, the antigen binding domain of the modified TCR complex can bind to multiple epitopes, e.g., multiple specificities.

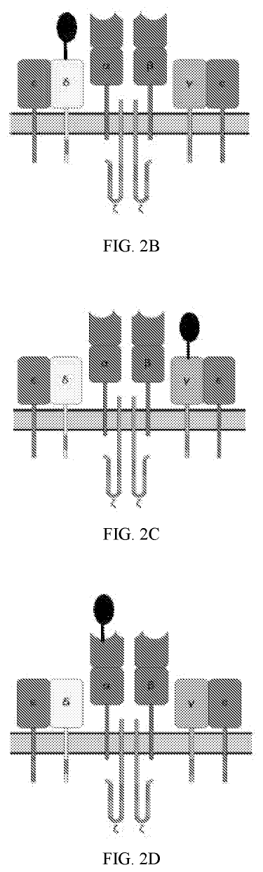

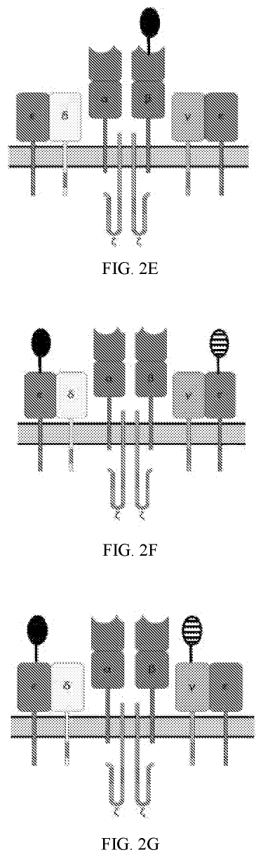

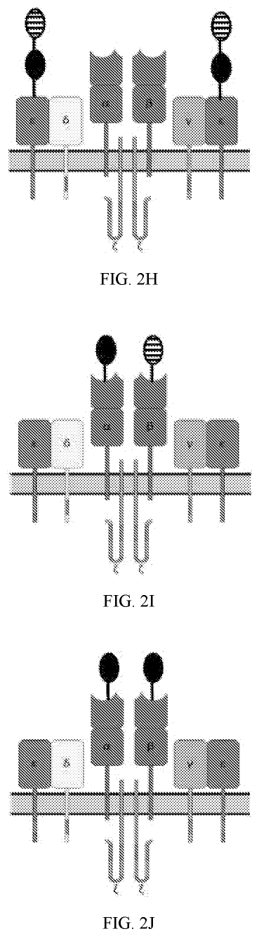

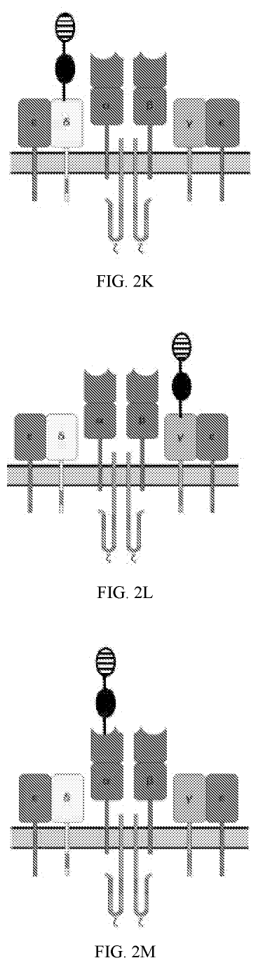

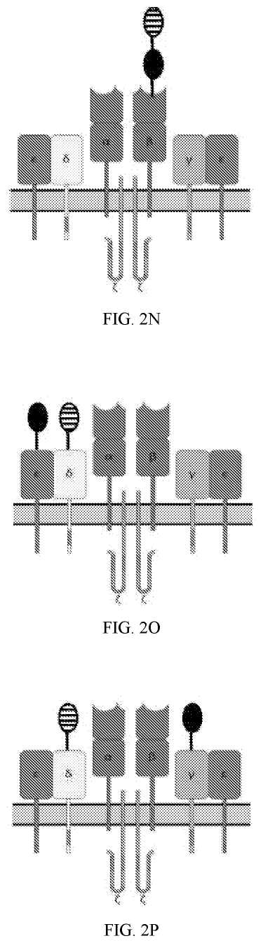

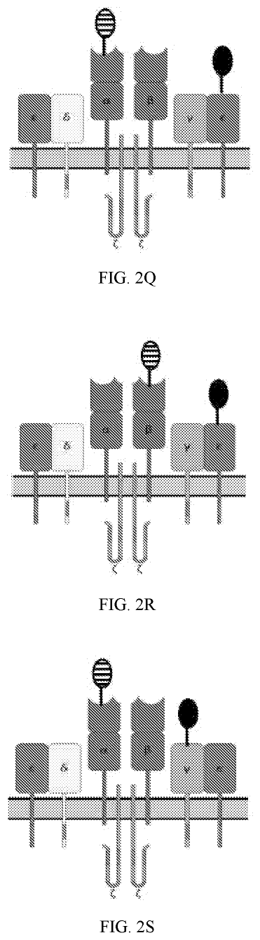

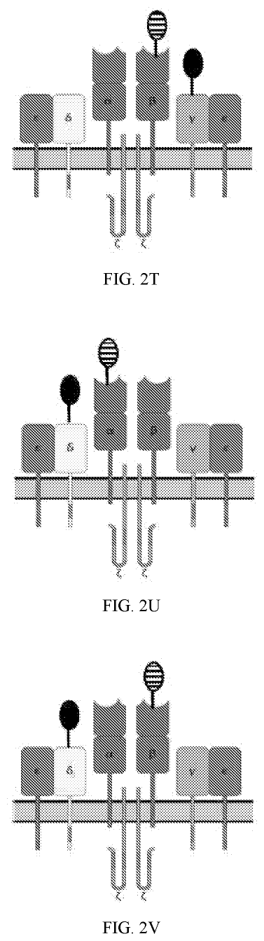

In some embodiments, a modified TCR complex comprises an antigen binding domain fused to CD3-epsilon chain, FIG. 2A. In some embodiments, a modified TCR complex comprises an antigen binding domain fused to a CD3-delta chain, FIG. 2B. In some embodiments, a modified TCR complex comprises an antigen binding domain fused to a CD3-gamma chain, FIG. 2C. In some embodiments, a modified TCR complex comprises an antigen binding domain fused to a TCR-alpha chain, FIG. 2D. In some embodiments, a modified TCR complex comprises an antigen binding domain fused to a TCR-beta chain, FIG. 2E. In some embodiments, a modified TCR complex comprises an antigen binding domain fused to a TCR-gamma chain. In some embodiments, a modified TCR complex comprises an antigen binding domain fused to a TCR-delta chain.

The modified TCR complex disclosed herein can comprise more than one antigen binding domain, for example at least 2 antigen binding domains (e.g., at least 3, 4, 5, 6, 7, 8, 9, or 10 antigen binding domains). In some embodiments, a modified TCR complex of a subject system comprises at least two antigen binding domains. The at least two antigen binding domains can be the same antigen binding domain. For example, the two antigen binding domains may be identical molecules capable of binding to the same ligand. The at least two antigen binding domains can be different antigen binding domains. For example, the two antigen binding domains may be different molecules capable of binding to the same or different ligand. In some cases, a modified TCR comprises a third antigen binding domain linked to (i) the second antigen binding domain, (ii) any of an alpha chain, a beta chain, a gamma chain and a delta chain of a TCR, (iii) an epsilon chain, delta chain, and/or a gamma chain of cluster of differentiation 3 (CD3), or (iv) CD3 zeta chain.

In some embodiments, a first antigen binding domain is fused to a first CD3-epsilon chain and a second antigen binding domain is fused to a second CD3-epsilon chain of a TCR complex, FIG. 2F. In some embodiments, a first antigen binding domain is fused to CD3-epsilon chain and a second antigen binding domain is fused to a CD3-gamma chain, FIG. 2G. In some embodiments, the first and second antigen binding domain are linked to the same chain. For example, a modified TCR complex disclosed herein can comprise a first antigen binding domain fused to a second antigen binding domain which in turn in fused to CD3-epsilon chain, FIG. 2H. In some embodiments, a first antigen binding domain is fused to TCR-alpha chain and a second antigen binding domain is fused to a TCR-beta chain. The first and the second antigen binding domains may be different antigen binding domains, as indicated by the black and black and white striped ovals (FIG. 2I). The first and the second antigen binding domains may be the same antigen binding domain, as indicated by the similarly shaded ovals (FIG. 2J).

In some embodiments, a modified TCR complex disclosed herein comprises a first antigen binding domain fused to a second antigen binding domain which in turn in fused to a CD3-delta chain, FIG. 2K. In some embodiments, a modified TCR complex disclosed herein comprises a first antigen binding domain fused to a second antigen binding domain which in turn in fused to a CD3-gamma chain, FIG. 2L. In some embodiments, a modified TCR complex disclosed herein comprises a first antigen binding domain fused to a second antigen binding domain which in turn in fused to a TCR-alpha chain, FIG. 2M. In some embodiments, a modified TCR complex disclosed herein comprises a first antigen binding domain fused to a second antigen binding domain which in turn in fused to a TCR-beta chain, FIG. 2N. The first and the second antigen binding domains may be different antigen binding domains. The first and the second antigen binding domains may be the same antigen binding domain.

In some embodiments, a modified TCR complex disclosed herein comprises a first antigen binding domain fused to CD3-epsilon chain and a second antigen binding domain fused to a CD3-delta chain, FIG. 2O. In some embodiments, a modified TCR complex disclosed herein comprises a first antigen binding domain fused to a CD3-delta chain and a second antigen binding domain fused to a CD3-gamma chain, FIG. 2P. In some embodiments, a modified TCR complex disclosed herein comprises a first antigen binding domain fused to a TCR-alpha chain and a second antigen binding domain fused to CD3-epsilon chain, FIG. 2Q. In some embodiments, a modified TCR complex disclosed herein comprises a first antigen binding domain fused to a TCR-beta chain and a second antigen binding domain fused to a CD3-epsilon chain, FIG. 2R. In some embodiments, a modified TCR complex disclosed herein comprises a first antigen binding domain fused to an alpha chain and a second antigen binding domain fused to a CD3-gamma chain, FIG. 2S. In some embodiments, a modified TCR complex disclosed herein comprises a first antigen binding domain fused to a TCR-beta chain and a second antigen binding domain fused to a CD3-gamma chain, FIG. 2T. In some embodiments, a modified TCR complex disclosed herein comprises a first antigen binding domain fused to a TCR-alpha chain and a second antigen binding domain fused to a CD3-delta chain, FIG. 2U. In some embodiments, a modified TCR complex disclosed herein comprises a first antigen binding domain fused to a beta chain and a second antigen binding domain fused to a delta chain, FIG. 2V.

In various embodiments of the aspects herein, a modified TCR complex comprises a TCR previously identified. In some cases, the TCR can be identified using whole-exomic sequencing. For example, a TCR can target a neoantigen or neoepitope that is identified by whole-exomic sequencing of a target cell. Alternatively, the TCR can be identified from autologous, allogenic, or xenogeneic repertoires. Autologous and allogeneic identification can entail a multistep process. In both autologous and allogeneic identification, dendritic cells (DCs) can be generated from CD14-selected monocytes and, after maturation, pulsed or transfected with a specific peptide. Peptide-pulsed DCs can be used to stimulate autologous or allogeneic immune cells, such as T cells. Single-cell peptide-specific T cell clones can be isolated from these peptide-pulsed T cell lines by limiting dilution. Subject TCRs of interest can be identified and isolated. Alpha, beta, gamma, and delta chains of a TCR of interest can be cloned, codon optimized, and encoded into a vector, for instance a lentiviral vector. In some embodiments, portions of the TCR can be replaced. For example, constant regions of a human TCR can be replaced with the corresponding murine regions. Replacement of human constant regions with corresponding murine regions can be performed to increase TCR stability. The TCR can also be identified with high or supraphysiologic avidity ex vivo. In some cases, a method of identifying a TCR can include immunizing transgenic mice that express the human leukocyte antigen (HLA) system with human tumor proteins to generate T cells expressing TCRs against human antigens (see e.g., Stanislawski et al., Circumventing tolerance to a human MDM2-derived tumor antigen by TCR gene transfer, Nature Immunology 2, 962-970 (2001)). An alternative approach can be allogeneic TCR gene transfer, in which tumor-specific T cells are isolated from a subject experiencing tumor remission and reactive TCR sequences can be transferred to T cells from another subject that shares the disease but may be non-responsive (de Witte, M. A., et al., Targeting self-antigens through allogeneic TCR gene transfer, Blood 108, 870-877(2006)). In some cases, in vitro technologies can be employed to alter a sequence of a TCR, enhancing their tumor-killing activity by increasing the strength of an interaction (avidity) of a weakly reactive tumor-specific TCR with target antigen (Schmid, D. A., et al., Evidence for a TCR affinity threshold delimiting maximal CD8 T cell function. J. Immunol. 184, 4936-4946 (2010)).

In another aspect, the present disclosure provides a system for inducing activity of an immune cell and/or a target cell. The system comprises (a) a chimeric antigen receptor (CAR) comprising a first antigen binding domain that exhibits specific binding to a first epitope, a transmembrane domain, and an intracellular signaling domain; and (b) a modified T cell receptor (TCR) complex disclosed herein.

In some embodiments, the system comprises (a) a chimeric antigen receptor (CAR) comprising a first antigen binding domain that exhibits specific binding to a first epitope, a transmembrane domain, and an intracellular signaling domain; and (b) a modified T cell receptor (TCR) complex comprising a second antigen binding domain which exhibits specific binding to a second epitope, wherein the second antigen binding domain is linked to at least one of (i) at least one TCR chain selected from an alpha chain, a beta chain, a gamma chain and a delta chain of a TCR; (ii) an epsilon chain, a delta chain, and/or a gamma chain of a cluster of differentiation 3 (CD3); or (iii) a CD3 zeta chain.

A chimeric antigen receptor (CAR) of a subject system can comprise a first antigen binding domain that exhibits specific binding to a first epitope. The first antigen binding domain can comprise any protein or molecule that can bind to an epitope. Non-limiting examples of the first antigen binding domain include, but are not limited to, a monoclonal antibody, a polyclonal antibody, a recombinant antibody, a human antibody, a humanized antibody, a murine antibody, or a functional derivative, variant or fragment thereof, including, but not limited to, a Fab, a Fab′, a F(ab′)₂, an Fv, a single-chain Fv (scFv), minibody, a diabody, and a single-domain antibody such as a heavy chain variable domain (VH), a light chain variable domain (VL) and a variable domain (V_{H}H) of camelid derived nanobody. In some embodiments, the first antigen binding domain comprises at least one of a Fab, a Fab′, a F(ab′)₂, an Fv, and a scFv. In some embodiments, the first antigen binding domain comprises an antibody mimetic. Antibody mimetics refer to molecules which can bind a target molecule with an affinity comparable to an antibody, and include single-chain binding molecules, cytochrome b562-based binding molecules, fibronectin or fibronectin-like protein scaffolds (e.g., adnectins), lipocalin scaffolds, calixarene scaffolds, A-domains and other scaffolds. In some embodiments, an antigen binding domain comprises a transmembrane receptor, or any derivative, variant, or fragment thereof. For example, an antigen binding domain can comprise at least a ligand binding domain of a transmembrane receptor.

In some embodiments, the antigen binding domain can comprise a scFv. A scFv can be derived from an antibody for which the sequences of the variable regions are known. In some embodiments, a scFv can be derived from an antibody sequence obtained from an available mouse hybridoma. A scFv can be obtained from whole-exomic sequencing of a tumor cell or primary cell. In some embodiments, a scFv can be altered. For instance, a scFv may be modified in a variety of ways. In some cases, a scFv can be mutated, so that the scFv may have higher affinity to its target. In some cases, the affinity of the scFv for its target can be optimized for targets expressed at low levels on normal tissues. This optimization can be performed to minimize potential toxicities, such as cytokine release syndrome. In other cases, the cloning of a scFv that has a higher affinity for the membrane bound form of a target can be preferable over its soluble form counterpart. This modification can be performed if some targets can also be detected in soluble form at different levels and their targeting can cause unintended toxicity, such as cytokine release syndrome.

In some embodiments, the first antigen binding domain of a CAR comprises an antigen binding molecules disclosed herein. In some embodiments, the first antigen binding domain comprises a single-domain antibody. In some embodiments, said single-domain antibody is an anti-BCMA sdAb. In some embodiments, the first antigen binding domain comprises a sequence having at least 80%, 85%, 90%, 95%, 96%, 97%, 98%, 99% or 100% identity to any one selected from SEQ ID NOs: 3-23 and 38-46. In some embodiments, said anti-BCMA sdAbs comprises a sequence of any one selected from SEQ ID NOs: 3-23.

In some embodiments, the antigen binding domain can comprise one member of an interacting pair. For example, the antigen binding domain may be one member, or a fragment thereof, of an interacting pair comprising a receptor and a ligand. Either the receptor or ligand, or fragments thereof, may be referred to as the antigen binding domain. The other member which is not referred to as the antigen binding domain can comprise the epitope to which the antigen binding domain specifically binds. In some embodiments, the first antigen binding domain and/or the second antigen binding domain comprises a receptor which specifically binds to a ligand. The receptor can comprise G-protein coupled receptors (GPCRs); integrin receptors; cadherin receptors; catalytic receptors including receptors possessing enzymatic activity and receptors which, rather than possessing intrinsic enzymatic activity, act by stimulating non-covalently associated enzymes (e.g., kinases); death receptors such as members of the tumor necrosis factor receptor (TNFR) superfamily; cytokine receptors; immune receptors; and the like. In some embodiments, the first antigen binding domain and/or the second antigen binding domain comprises a ligand which is bound by a receptor.

An antigen binding domain of a CAR of a subject system can be linked to an intracellular signaling domain via a transmembrane domain. A transmembrane domain can be a membrane spanning segment. A transmembrane domain of a subject CAR can anchor the CAR to the plasma membrane of a cell, for example an immune cell. In some embodiments, the membrane spanning segment comprises a polypeptide. The membrane spanning polypeptide linking the antigen binding domain and the intracellular signaling domain of the CAR can have any suitable polypeptide sequence. In some cases, the membrane spanning polypeptide comprises a polypeptide sequence of a membrane spanning portion of an endogenous or wild-type membrane spanning protein. In some embodiments, the membrane spanning polypeptide comprises a polypeptide sequence having at least 1 (e.g., at least 2, 3, 4, 5, 6, 7, 8, 9, 10 or greater) of an amino acid substitution, deletion, and insertion compared to a membrane spanning portion of an endogenous or wild-type membrane spanning protein. In some embodiments, the membrane spanning polypeptide comprises a non-natural polypeptide sequence, such as the sequence of a polypeptide linker. The polypeptide linker may be flexible or rigid. The polypeptide linker can be structured or unstructured. In some embodiments, the membrane spanning polypeptide transmits a signal from an extracellular region of a cell to an intracellular region, for via the antigen binding domain. A native transmembrane portion of CD28 can be used in a CAR. In other cases, a native transmembrane portion of CD8 alpha can also be used in a CAR.

The intracellular signaling domain of a CAR of a subject system can comprise a signaling domain, or any derivative, variant, or fragment thereof, involved in immune cell signaling. The intracellular signaling domain of a CAR can induce activity of an immune cell comprising the CAR. The intracellular signaling domain can transduce the effector function signal and direct the cell to perform a specialized function. The signaling domain can comprise signaling domains of other molecules. While usually the signaling domain of another molecule can be employed in a CAR, in many cases it is not necessary to use the entire chain. In some cases, a truncated portion of the signaling domain is used in a CAR.

In some embodiments, the intracellular signaling domain comprises multiple signaling domains involved in immune cell signaling, or any derivatives, variants, or fragments thereof. For example, the intracellular signaling domain can comprise at least 2 immune cell signaling domains, e.g., at least 2, 3, 4, 5, 7, 8, 9, or 10 immune cell signaling domains. An immune cell signaling domain can be involved in regulating primary activation of the TCR complex in either a stimulatory way or an inhibitory way. The intracellular signaling domain may be that of a T-cell receptor (TCR) complex. The intracellular signaling domain of a subject CAR can comprise a signaling domain of an Fcγ receptor (FcγR), an Fcε receptor (FcεR), an Fcα receptor (FcαR), neonatal Fc receptor (FcRn), CD3, CD3 ζ, CD3 γ, CD3 δ, CD3 ε, CD4, CD5, CD8, CD21, CD22, CD28, CD32, CD40L (CD154), CD45, CD66d, CD79a, CD79b, CD80, CD86, CD278 (also known as ICOS), CD247 ζ, CD247 η, DAP10, DAP12, FYN, LAT, Lck, MAPK, MHC complex, NFAT, NF-κB, PLC-γ, iC3b, C3dg, C3d, and Zap70. In some embodiments, the signaling domain includes an immunoreceptor tyrosine-based activation motif or ITAM. A signaling domain comprising an ITAM can comprise two repeats of the amino acid sequence YxxL/I separated by 6-8 amino acids, wherein each x is independently any amino acid, producing the conserved motif YxxL/Ix₍₆₋₈₎YxxL/I. A signaling domain comprising an ITAM can be modified, for example, by phosphorylation when the antigen binding domain is bound to an epitope. A phosphorylated ITAM can function as a docking site for other proteins, for example proteins involved in various signaling pathways. In some embodiments, the primary signaling domain comprises a modified ITAM domain, e.g., a mutated, truncated, and/or optimized ITAM domain, which has altered (e.g., increased or decreased) activity compared to the native ITAM domain.

In some embodiments, the intracellular signaling domain of a subject CAR comprises an FcγR signaling domain (e.g., ITAM). The FcγR signaling domain can be selected from FcγRI (CD64), FcγRIIA (CD32), FcγRIIB (CD32), FcγRIIIA (CD16a), and FcγRIIIB (CD16b). In some embodiments, the intracellular signaling domain comprises an FcεR signaling domain (e.g., ITAM). The FcεR signaling domain can be selected from FcεRI and FcεRII (CD23). In some embodiments, the intracellular signaling domain comprises an FcαR signaling domain (e.g., ITAM). The FcαR signaling domain can be selected from FcαRI (CD89) and Fcα/μR. In some embodiments, the intracellular signaling domain comprises a CD3 ζ signaling domain. In some embodiments, the primary signaling domain comprises an ITAM of CD3 ζ.

In some embodiments, an intracellular signaling domain of a subject CAR comprises an immunoreceptor tyrosine-based inhibition motif or ITIM. A signaling domain comprising an ITIM can comprise a conserved sequence of amino acids (S/I/V/LxYxxI/V/L) that is found in the cytoplasmic tails of some inhibitory receptors of the immune system. A primary signaling domain comprising an ITIM can be modified, for example phosphorylated, by enzymes such as a Src kinase family member (e.g., Lck). Following phosphorylation, other proteins, including enzymes, can be recruited to the ITIM. These other proteins include, but are not limited to, enzymes such as the phosphotyrosine phosphatases SUP-1 and SHP-2, the inositol-phosphatase called SHIP, and proteins having one or more SH2 domains (e.g., ZAP70). A intracellular signaling domain can comprise a signaling domain (e.g., ITIM) of BTLA, CD5, CD31, CD66a, CD72, CMRF35H, DCIR, EPO-R, FcγRIIB (CD32), Fc receptor-like protein 2 (FCRL2), Fc receptor-like protein 3 (FCRL3), Fc receptor-like protein 4 (FCRL4), Fc receptor-like protein 5 (FCRL5), Fc receptor-like protein 6 (FCRL6), protein G6b (G6B), interleukin 4 receptor (IL4R), immunoglobulin superfamily receptor translocation-associated 1(IRTA1), immunoglobulin superfamily receptor translocation-associated 2 (IRTA2), killer cell immunoglobulin-like receptor 2DL1 (KIR2DL1), killer cell immunoglobulin-like receptor 2DL2 (KIR2DL2), killer cell immunoglobulin-like receptor 2DL3 (KIR2DL3), killer cell immunoglobulin-like receptor 2DL4 (KIR2DL4), killer cell immunoglobulin-like receptor 2DL5 (KIR2DL5), killer cell immunoglobulin-like receptor 3DL1 (KIR3DL1), killer cell immunoglobulin-like receptor 3DL2 (KIR3DL2), leukocyte immunoglobulin-like receptor subfamily B member 1 (LIR1), leukocyte immunoglobulin-like receptor subfamily B member 2 (LIR2), leukocyte immunoglobulin-like receptor subfamily B member 3 (LIR3), leukocyte immunoglobulin-like receptor subfamily B member 5 (LIR5), leukocyte immunoglobulin-like receptor subfamily B member 8 (LIR8), leukocyte-associated immunoglobulin-like receptor 1 (LAIR-1), mast cell function-associated antigen (MAFA), NKG2A, natural cytotoxicity triggering receptor 2 (NKp44), NTB-A, programmed cell death protein 1 (PD-1), PILR, SIGLECL1, sialic acid binding Ig like lectin 2 (SIGLEC2 or CD22), sialic acid binding Ig like lectin 3 (SIGLEC3 or CD33), sialic acid binding Ig like lectin 5 (SIGLEC5 or CD170), sialic acid binding Ig like lectin 6 (SIGLEC6), sialic acid binding Ig like lectin 7 (SIGLEC7), sialic acid binding Ig like lectin 10 (SIGLEC10), sialic acid binding Ig like lectin 11 (SIGLEC11), sialic acid binding Ig like lectin 4 (SIGLEC4), sialic acid binding Ig like lectin 8 (SIGLEC8), sialic acid binding Ig like lectin 9 (SIGLEC9), platelet and endothelial cell adhesion molecule 1 (PECAM-1), signal regulatory protein (SIRP 2), and signaling threshold regulating transmembrane adaptor 1 (SIT). In some embodiments, the intracellular signaling domain comprises a modified ITIM domain, e.g., a mutated, truncated, and/or optimized ITIM domain, which has altered (e.g., increased or decreased) activity compared to the native ITIM domain.

In some embodiments, the intracellular signaling domain comprises at least 2 ITAM domains (e.g., at least 3, 4, 5, 6, 7, 8, 9, or 10 ITAM domains). In some embodiments, the intracellular signaling domain comprises at least 2 ITIM domains (e.g., at least 3, 4, 5, 6, 7, 8, 9, or 10 ITIM domains) (e.g., at least 2 primary signaling domains). In some embodiments, the intracellular signaling domain comprises both ITAM and ITIM domains.

In some cases, the intracellular signaling domain of a subject CAR can include a co-stimulatory domain. In some embodiments, a co-stimulatory domain, for example from co-stimulatory molecule, can provide co-stimulatory signals for immune cell signaling, such as signaling from ITAM and/or ITIM domains, e.g., for the activation and/or deactivation of immune cell activity. In some embodiments, a costimulatory domain is operable to regulate a proliferative and/or survival signal in the immune cell. In some embodiments, a co-stimulatory signaling domain comprises a signaling domain of a MHC class I protein, MHC class II protein, TNF receptor protein, immunoglobulin-like protein, cytokine receptor, integrin, signaling lymphocytic activation molecule (SLAM protein), activating NK cell receptor, BTLA, or a Toll ligand receptor. In some embodiments, the costimulatory domain comprises a signaling domain of a molecule selected from the group consisting of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD5, CD53, CD58/LFA-3, CD69, CD7, CD8 α, CD8 β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6. In some embodiments, the intracellular signaling domain comprises multiple costimulatory domains, for example at least two, e.g., at least 3, 4, or 5 costimulatory domains. Co-stimulatory signaling regions may provide a signal synergistic with the primary effector activation signal and can complete the requirements for activation of a T cell. In some embodiments, the addition of co-stimulatory domains to the CAR can enhance the efficacy and persistence of the immune cells provided herein.

Examples of costimulatory signaling domains are provided in Table 8.

TABLE 8
Intracellular co-stimulatory signaling domains

| Gene | | | NCBI number | | | Location |
|---|---|---|---|---|---|---|
| Symbol | Abbreviation | Name | (GRCh38.p2) | Start | Stop | in genome |
| CD27 | CD27; T14; | CD27 molecule | 939 | 6444885 | 6451718 | 12p13 |
| | S152; Tp55; | | | | | |
| | TNFRSF7; | | | | | |
| | S152. LPFS2 | | | | | |
| CD28 | Tp44; CD28; | CD28 molecule | 940 | 203706475 | 203738912 | 2q33 |
| | CD28 antigen | | | | | |
| TNFRSF9 | ILA; 4-1BB; | tumor necrosis | 3604 | 7915871 | 7943165 | 1p36 |
| | CD137; | factor receptor | | | | |
| | CDw137 | superfamily, | | | | |
| | | member 9 | | | | |
| TNFRSF4 | OX40; ACT35; | tumor necrosis | 7293 | 1211326 | 1214638 | 1p36 |
| | CD134; IMD16; | factor receptor | | | | |
| | TXGP1L | superfamily, | | | | |
| | | member 4 | | | | |
| TNFRSF8 | CD30; Ki-1; | tumor necrosis | 943 | 12063330 | 12144207 | 1p36 |
| | D1S166E | factor receptor | | | | |
| | | superfamily, | | | | |
| | | member 8 | | | | |
| CD40LG | IGM; IMD3; | CD40 ligand | 959 | 136648177 | 136660390 | Xq26 |
| | TRAP; gp39; | | | | | |
| | CD154; CD40L; | | | | | |
| | HIGM1; T-BAM; | | | | | |
| | TNFSF5; | | | | | |
| | hCD40L | | | | | |
| ICOS | AILIM; CD278; | inducible T-cell | 29851 | 203936731 | 203961579 | 2q33 |
| | CVID1 | co-stimulator | | | | |
| ITGB2 | LAD; CD18; | integrin, beta 2 | 3689 | 44885949 | 44928873 | 21q22.3 |
| | MF17; MF17; | (complement | | | | |
| | LCAMB; LFA-1; | component 3 | | | | |
| | MAC-1 | receptor 3 and | | | | |
| | | 4 subunit) | | | | |
| CD2 | T11; SRBC; | CD2 molecule | 914 | 116754435 | 116769229 | 1p13.1 |
| | LFA-2 | | | | | |
| CD7 | GP40; TP41; | CD7 molecule | 924 | 82314865 | 82317604 | 17q25.2- |
| | Tp40; LEU-9 | | | | | q25.3 |
| KLRC2 | NKG2C; | killer cell lectin- | 3822 | 10430599 | 10435993 | 12p13 |
| | CD159c; | like receptor | | | | |
| | NKG2-C | subfamily C, | | | | |
| | | member 2 | | | | |
| TNFRSF18 | AITR; GITR; | tumor necrosis | 8784 | 1203508 | 1206709 | 1p36.3 |
| | CD357; GITR-D | factor receptor | | | | |
| | | superfamily, | | | | |
| | | member 18 | | | | |
| TNFRSF14 | TR2; ATAR; | tumor necrosis | 8764 | 2556365 | 2565622 | 1p36.32 |
| | HVEA; HVEM; | factor receptor | | | | |
| | CD270; | superfamily, | | | | |
| | LIGHTR | member 14 | | | | |
| HAVCR1 | TIM; KIM1; | hepatitis A | 26762 | 156979480 | 157069527 | 5q33.2 |
| | TIM1; CD365; | virus cellular | | | | |
| | HAVCR; KIM-1; | receptor 1 | | | | |
| | TIM-1; TIMD1; | | | | | |
| | TIMD-1; | | | | | |
| | HAVCR-1 | | | | | |
| LGALS9 | HUAT; | lectin, | 3965 | 27631148 | 27649560 | 17q11.2 |
| | LGALS9A, | galactoside- | | | | |
| | Galectin-9 | binding, | | | | |
| | | soluble, 9 | | | | |
| CD83 | BL11; HB15 | CD83 | 9308 | 14117256 | 14136918 | 6p23 |
| | | molecule | | | | |

As an example, a CAR can comprise a CD3 zeta-chain (sometimes referred to as a 1st generation CAR). As another example, a CAR can comprise a CD-3 zeta-chain and a single co-stimulatory domain (for example, CD28 or 4-1B) (sometimes referred to as a 2nd generation CAR). As another example, a CAR can comprise a CD-3 zeta-chain and two co-stimulatory domains (CD28/OX40 or CD28/4-1BB) (sometimes referred to as a 3rd generation CAR). Together with co-receptors such as CD8, these signaling moieties can produce downstream activation of kinase pathways, which support gene transcription and functional cellular responses.

In some embodiments, a subject CAR can comprise a hinge or a spacer. The hinge or the spacer can refer to a segment between the antigen binding domain and the transmembrane domain. In some embodiments, a hinge can be used to provide flexibility to an antigen binding domain, e.g., scFv. In some embodiments, a hinge can be used to detect the expression of a CAR on the surface of a cell, for example when antibodies to detect the scFv are not functional or available. In some cases, the hinge is derived from an immunoglobulin molecule and may require optimization depending on the location of the first epitope or second epitope on the target. In some cases, a hinge may not belong to an immunoglobulin molecule but instead to another molecule such the native hinge of a CD8 alpha molecule. A CD8 alpha hinge can contain cysteine and proline residues which many play a role in the interaction of a CD8 co-receptor and MHC molecule. In some embodiments, a cysteine and proline residue can influence the performance of a CAR and may therefore be engineered to influence a CAR performance.

A hinge can be of any suitable length. In some embodiments, a CAR's hinge can be size tunable and can compensate to some extent in normalizing the orthogonal synapse distance between a CAR expressing cell and a target cell. This topography of the immunological synapse between the CAR expressing cell and target cell can also define a distance that cannot be functionally bridged by a CAR due to a membrane-distal epitope on a cell-surface target molecule that, even with a short hinge CAR, cannot bring the synapse distance in to an approximation for signaling. Likewise, membrane-proximal CAR target antigen epitopes have been described for which signaling outputs are only observed in the context of a long hinge CAR. A hinge disclosed herein can be tuned according to the single chain variable fragment region that can be used.

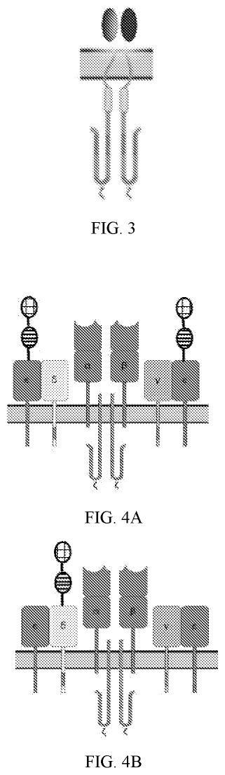

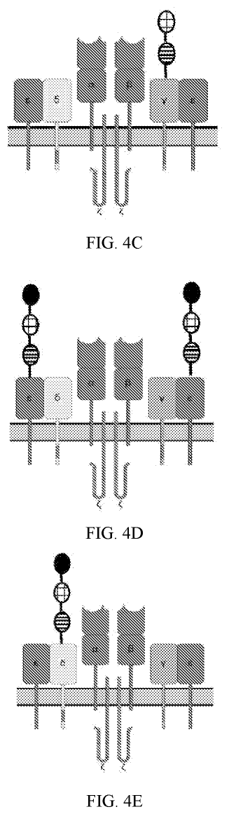

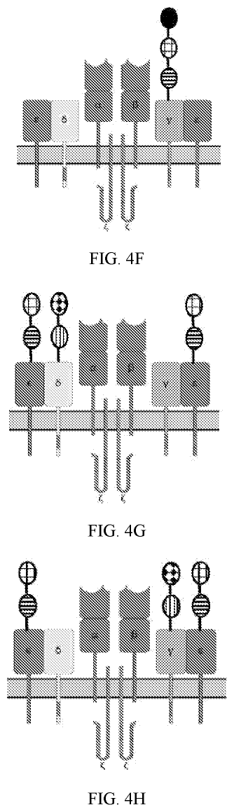

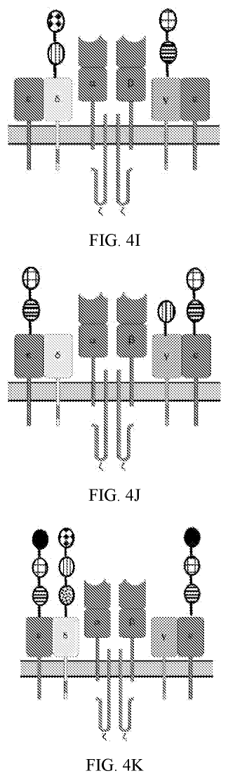

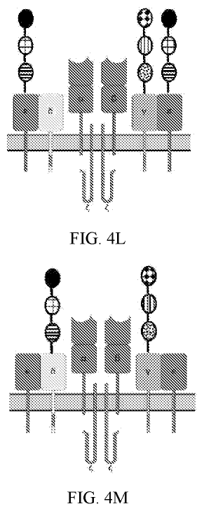

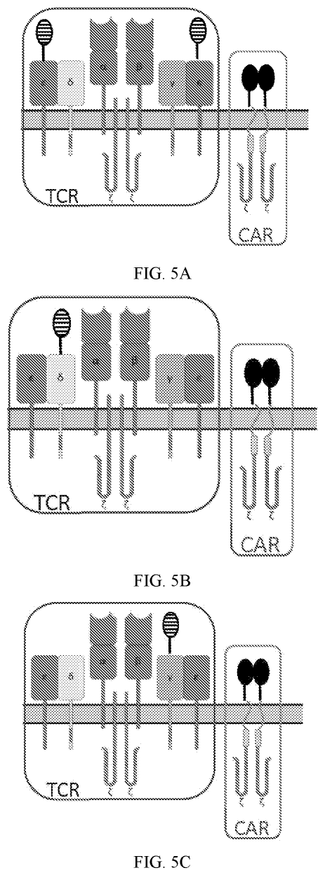

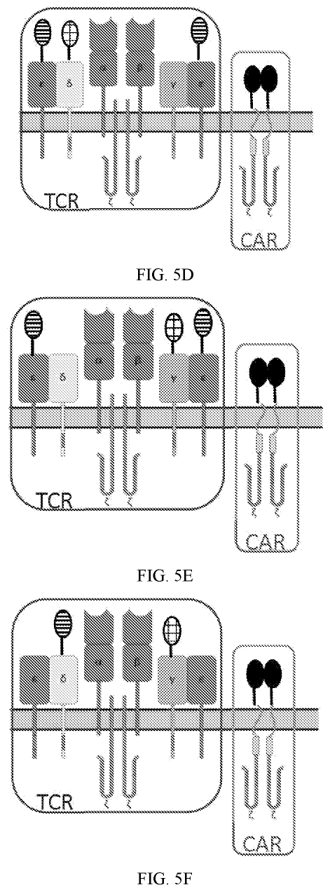

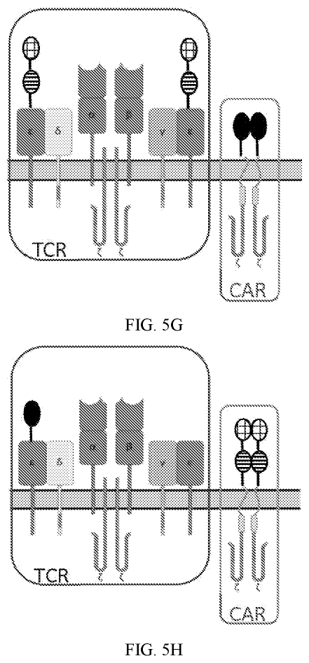

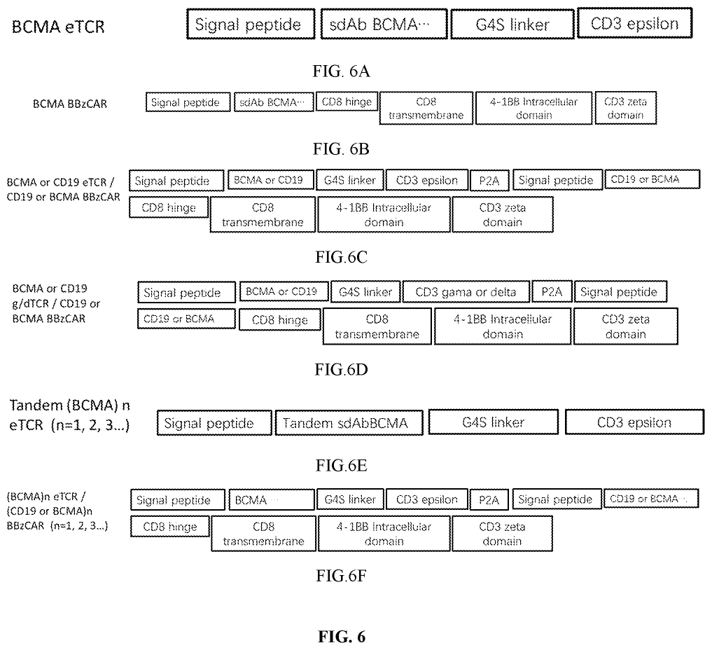

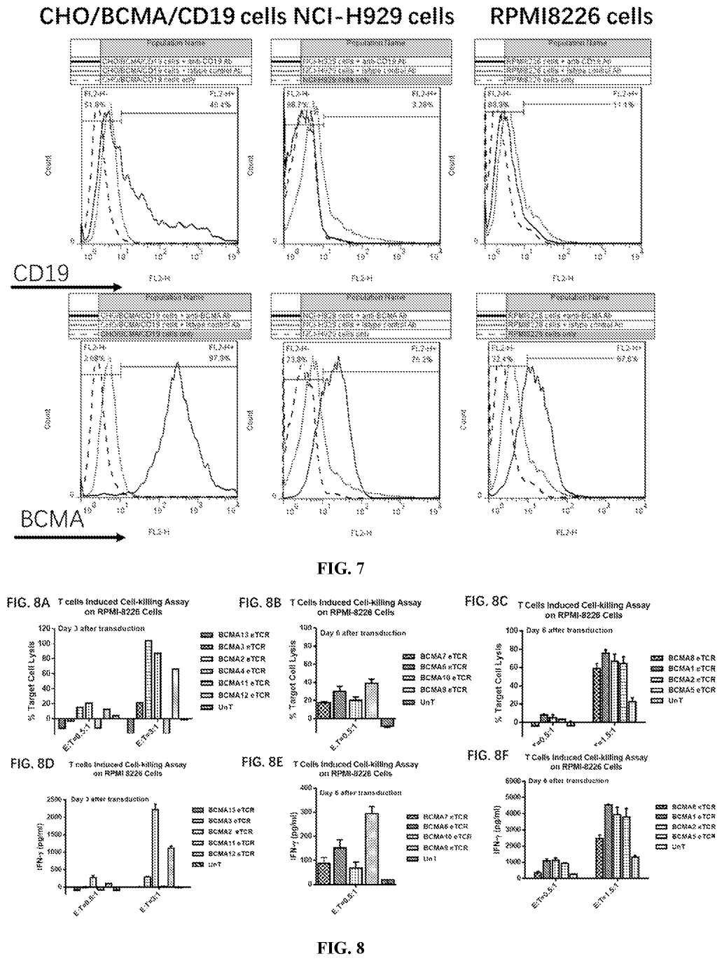

As an example, a CAR can comprise an extracellular antigen binding domain, a transmembrane domain, and an intracellular signaling domain, is illustrated in FIG. 3. A CAR may generally comprise an antigen binding domain derived from single chain antibody, hinge domain (H) or spacer, transmembrane domain (TM) providing anchorage to plasma membrane, and signaling domains responsible of T-cell activation. A CAR can comprise a immune cell signaling domain, such as a CD3ζ-chain. A CAR can comprise an immune cell signaling domains and a first costimulatory domain, such as CD3ζ-chain and 4-1BB. A CAR can comprise an immune cell signaling domain and at least two costimulatory domains, such as CD3ζ-chain, 4-1BB, and OX40. In some embodiments, a universal CAR can also be comprised in a system. A universal CAR can comprise an intracellular signaling domain fused to a protein domain that binds a tag (e.g., fluorescein isothiocyanate or biotin) on a monoclonal antibody. Various combinations of immune cell signaling domains and costimulatory domains may be utilized in a subject CAR. In some embodiments, immune cell signaling domains may be from CD3, CD4, and/or CD8. Costimulatory domains can be from 4-1BB, OX40, CD28, and the like.

In some embodiments, a CAR of a subject system of the present disclosure can comprise one or more additional antigen binding domains exhibit specific binding to one or more additional epitopes. For example, a CAR of a subject system can comprise at least two antigen binding domains (e.g., at least 3, 4, 5, 6, 7, 8, 9, or 10 antigen binding domains). In some embodiments, said at least two antigen binding domains of the CAR are linked in tandem. In some embodiments, said at least two antigen binding domains can be the same antigen binding domain. For example, the at least two antigen binding domains may be identical molecules capable of binding to the same epitope. In some embodiments, said at least two antigen binding domains can be different antigen binding domains. For example, the at least two antigen binding domains may be different molecules capable of binding to different epitopes on one or more antigen.

The antigen binding domain of a subject CAR and a modified TCR complex of a subject system can bind to epitopes that are present on different antigens. In some cases, the antigen binding domains of the CAR and the modified TCR complex of the subject system bind epitopes present on a common antigen. In some embodiments, a first epitope and a second epitope can be the same epitope. In some embodiments, a first epitope and a second epitope can be different epitopes.

The first epitope and/or the second epitope may be present on one or more cell surface antigens. The one or more cell surface antigens can be tyrosine kinase receptors, serine kinase receptors, histidine kinase receptor, G-protein coupled receptors (GPCR), and the like

The first epitope and/or the second epitope may be present on an immune checkpoint receptor or immune checkpoint receptor ligand. In some embodiments, the immune checkpoint receptor or immune checkpoint receptor ligand can be PD-1, PD-L1, PD-L2, CTLA-4, TIM-3, LAG3, TIGIT, BLTA, CD47 or CD40.

The first epitope and/or the second epitope may be present on a cytokine or a cytokine receptor. A cytokine receptor can be, for example, CCR2b, CXCR2 (CXCL1 receptor), CCR4 (CCL17 receptor), Gro-a, IL-2, IL-7, IL-15, IL-21, IL-12, Heparanase, CD137L, LEM, Bcl-2, CCL17, CCL19 or CCL2.

The first epitope and/or the second epitope can be present on a tumor-associated antigen. The epitope may be, for instance a tumor epitope. A tumor-associated antigen can be selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFαRII, TGFβRII, TP1, TRAG-3, TRG, TRP-1, TRP-2, TRP-2/INT2, TRP-2-6b, Tyrosinase, VEGF-R2, WT1, α-folate receptor, and κ-light chain. In some embodiments, a first epitope and/or a second epitope can be EGFR, EGFRvIII, GPC3, GPC-2, DLL3, BCMA, CD19, CD20, CD22, CD123, CLL-1, CD30, CD33, HER2, MSLN, PSMA, CEA, GD2, IL13Rα2, CAIX, L1-CAM, CA125, CD133, FAP, CTAG1B, MUC1, FR-α, CD70, CD171, ROR1, and any combination thereof.

In some embodiments, the first epitope or the second epitope is present on BCMA. In some embodiments, the first epitope and the second epitope are both present on BCMA. In some embodiments, the first epitope and the second epitope are the same epitope of BCMA. In some embodiments, the first epitope and the second epitope are different epitopes of BCMA.

The first epitope and/or the second epitope may be present on a neoantigen. The first epitope and/or the second epitope may be a neoepitope.

Neoantigens and neoepitopes generally refer to tumor-specific mutations that in some cases trigger an antitumor T cell response. For example, these endogenous mutations can be identified using a whole-exomic-sequencing approach. Tran E, et al., “Cancer immunotherapy based on mutation-specific CD4+ T cells in a patient with epithelial cancer,” Science 344: 641-644 (2014). An antigen binding domain, for example, that of a subject CAR or a modified TCR complex can exhibit specific binding to a tumor-specific neo-antigen. Neoantigens bound by antigen binding domains of a CAR or modified TCR complex can be expressed on a target cell, and for example, are neoantigens and neoeptiopes encoded by mutations in any endogenous gene. In some cases, the first and/or second antigen binding domains bind a neoantigen or neoepitope encoded by a mutated gene. The gene can be selected from the group consisting of: ABL1, ACOl 1997, ACVR2A, AFP, AKT1, ALK, ALPPL2, ANAPC1, APC, ARID1A, AR, AR-v7, ASCL2, β2M, BRAF, BTK, C15ORF40, CDH1, CLDN6, CNOT1, CT45A5, CTAG1B, DCT, DKK4, EEF1B2, EEF1DP3, EGFR, EIF2B3, env, EPHB2, ERBB3, ESR1, ESRP1, FAM11 IB, FGFR3, FRG1B, GAGE1, GAGE 10, GATA3, GBP3, HER2, IDH1, JAK1, KIT, KRAS, LMAN1, MABEB 16, MAGEA1, MAGEA10, MAGEA4, MAGEA8, MAGEB 17, MAGEB4, MAGEC1, MEK, MLANA, MLL2, MMP13, MSH3, MSH6, MYC, NDUFC2, NRAS, NY-ESO, PAGE2, PAGE5, PDGFRa, PIK3CA, PMEL, pol protein, POLE, PTEN, RAC1, RBM27, RNF43, RPL22, RUNX1, SEC31A, SEC63, SF3B 1, SLC35F5, SLC45A2, SMAP1, SMAP1, SPOP, TFAM, TGFBR2, THAP5, TP53, TTK, TYR, UBR5, VHL, and XPOT.

In some embodiments, a first epitope and/or a second epitope which can be bound by the first and/or second antigen binding domain can be present on a stroma. Stroma generally refers to tissue which, among other things, provides connective and functional support of a biological cell, tissue, or organ. A stroma can be that of the tumor microenvironment. The first epitope and/or second epitope may be present on a stromal antigen. Such an antigen can be on the stroma of the tumor microenvironment. Neoantigens and neoepitopes, for example, can be present on tumor endothelial cells, tumor vasculature, tumor fibroblasts, tumor pericytes, tumor stroma, and/or tumor mesenchymal cells. Example antigens include, but are not limited to, CD34, MCSP, FAP, CD31, PCNA, CD117, CD40, MMP4, and Tenascin.

In some embodiments, a first epitope and/or a second epitope can be present on an antigen presented by a major histocompatibility complex (MHC). An MHC can be human leukocyte antigen (HLA) class I or class II. An HLA can be HLA-A, HLA-B, HLA-C, HLA-HLA-E, HLA-F, HLA-G, HLA-DP, HLA-DQ, HLA-DR, HLA-DM, or HLA-DO. In some embodiments, a first epitope/and or a second epitope can be present on HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, or HLA-A*24, HLA-B*27, HLA-B*35, HLA-B*48, HLA-B*55, and the like.

In some embodiments, a first epitope and/or a second epitope can be soluble (e.g., not bound to a cell). In some cases, the antigen can be soluble, e.g., a soluble antigen. The first epitope and/or the second epitope may be present on a universal antigen. In some cases, the antigen binding domain of a subject CAR and/or a modified TCR complex each can bind to multiple epitopes, e.g., multiple specificities.

In some embodiments, a first epitope and a second epitope can be the same epitope.

In some embodiments, binding of at least one antigen binding domain to its epitope can activate an immune cell activity of an immune cell expressing the modified TCR complex of the present disclosure. In some embodiments, binding of two or more antigen binding domains to their epitopes can activate an immune cell activity of an immune cell expressing the modified TCR complex of the present disclosure. In some embodiments, binding of the first antigen binding domain to the first epitope or binding of the second antigen binding domain to the second epitope can activate an immune cell activity of an immune cell expressing the subject system. In some cases, binding of the first antigen binding domain to the first epitope and binding of the second antigen binding domain to the second epitope activates an immune cell activity of an immune cell expressing the system.

In some embodiments, a system for inducing activity of an immune cell and/or a target cell can comprise more than two antigen binding domains. For example, a system can comprise a first, second, third, fourth, fifth, sixth, seventh, eighth, ninth, or tenth or even more antigen binding domains. In some embodiments, binding of the third antigen binding domain to a third epitope activates an immune cell activity of an immune cell expressing the system. In some embodiments, binding of the first antigen binding domain to the first epitope, binding of the second antigen binding domain to the second epitope, and binding of the third antigen binding domain to the third epitope activates an immune cell activity of an immune cell expressing the system. Any number of antigen binding domains can be used in systems of the present disclosure, and the number of antigen binding domains is not limited to one, two or three.

In some embodiments, two or more antigen binding domains of the subject system are linked to, optionally in tandem, (i) at least one TCR chain selected from an alpha chain, a beta chain, a gamma chain and a delta chain of a T cell receptor, (ii) an epsilon chain, a delta chain, and/or a gamma chain of cluster of differentiation 3 (CD3), (iii) a CD3 zeta chain, and wherein binding of the two more antigen binding domains to their respective epitopes activates an immune cell activity of an immune cell expressing the system. Where desired, the two or more antigen binding domains are linked to separate chains of the TCR complex. Alternatively, the two or more antigen binding domains are linked to one chain of the TCR complex. In some embodiments of the subject system, the two or more antigen binding domains are linked in tandem on the epsilon chain, the delta chain, and/or the gamma chain of cluster of differentiation 3 (CD3). In some embodiments, two or more antigen binding domains of the subject system are linked to, optionally in tandem to the CAR the subject system.

The immune cell activity that is activated in the immune cell expressing the modified TCR complex and/or the system of the present disclosure can be any of a variety of cellular activities. In some embodiments, the immune cell activity is selected from the group consisting of clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell.

In some embodiments, the immune cell activity comprises clonal expansion of the immune cell. Clonal expansion can comprise the generation of daughter cells arising from the immune cell. In a clonal expansion, progeny of the immune cell can comprise a modified TCR complex and/or a system provided herein. In a clonal expansion, progeny of the immune cell can comprise a CAR provided herein. In a clonal expansion, progeny of the immune cell can comprise a modified TCR complex provided herein. In a clonal expansion, progeny of the immune cell can comprise the CAR and the TCR provided herein. Clonal expansion of an immune cell comprising a modified TCR complex and/or a system provided herein can be greater than that of a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. Clonal expansion of an immune cell comprising a modified TCR complex and/or a system provided herein can be about 5 fold to about 10 fold, about 10 fold to about 20 fold, about 20 fold to about 30 fold, about 30 fold to about 40 fold, about 40 fold to about 50 fold, about 50 fold to about 60 fold, about 60 fold to about 70 fold, about 70 fold to about 80 fold, about 80 fold to about 90 fold, about 90 fold to about 100 fold, about 100 fold to about 200 fold, about 200 fold to about 300 fold, about 300 fold to about 400 fold, about 400 fold to about 500 fold, about 500 fold to about 600 fold, about 600 fold to about 700 fold greater than a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, clonal expansion can comprise quantifying the number of immune cells. Quantifying a number of immune cells can comprise, flow cytometry, Trypan Blue exclusion, and/or hemocytometry.

In some embodiments, the immune cell activity comprises cytokine release by the immune cell. In some embodiments, the immune cell activity comprises release of intercellular molecules, metabolites, chemical compounds or combinations thereof. Cytokine release by the immune cell can comprise the release of IL-1, IL-2, IL-4, IL-5, IL-6, IL-13, IL-17, IL-21, IL-22, IFNγ, TNFα, CSF, TGFβ, granzyme, and the like. In some embodiments, cytokine release may be quantified using HTRF, flow cytometry, western blot, and the like. Cytokine release by an immune cell comprising a modified TCR complex and/or a system provided herein can be greater than that of a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. An immune cell comprising a modified TCR complex and/or a system provided herein can generate from about 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 20 fold, 30 fold, 40 fold, 50 fold, 60 fold, 70 fold, 80 fold, 90 fold, 100 fold, 150 fold, 200 fold, 250 fold, or over 300 fold greater cytokine release as compared to a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, cytokine release can be quantified, in vitro or in vivo.

In some embodiments, the immune cell activity comprises cytotoxicity of the immune cell. In some examples, the modified TCR complex, the subject systems and compositions of the present disclosure, when expressed in an immune cell, can be used for killing a target cell. An immune cell or population of immune cells expressing a modified TCR complex and/or a subject system can induce death of a target cell. Killing of a target cell can be useful for a variety of applications, including, but not limited to, treating a disease or disorder in which a cell population is desired to be eliminated or its proliferation desired to be inhibited. Cytotoxicity can refer to the killing of the target cell. Cytotoxicity can also refer to the release of cytotoxic cytokines, for example IFNγ or granzyme, by the immune cell. In some cases, a modified TCR complex and/or a subject system expressed in immune cells can alter the (i) release of cytotoxins such as perforin, granzymes, and granulysin and/or (ii) induction of apoptosis via Fas-Fas ligand interaction between the T cells and target cells, thereby triggering the destruction of target cells. In some embodiments, cytotoxicity can be quantified by a cytotoxicity assay including, a co-culture assay, ELISPOT, chromium release cytotoxicity assay, and the like. Cytotoxicity of an immune cell comprising a modified TCR complex and/or a system provided herein can be greater than that of a comparable immune cell lacking the modified TCR complex and/or the system, a comparable immune cell lacking one or more components of the modified TCR complex and/or the system (e.g., CAR, modified TCR complex), and/or a comparable immune cell in which one only one of the first and second antigen binding domains is bound to their respective epitopes. An immune cell comprising a modified TCR complex and/or a system provided herein can be about 5% 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% more cytotoxic to target cells as compared to a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. An immune cell comprising a modified TCR complex and/or a system provided herein can induce death of target cells that is at least 5% 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater than that of a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, an immune cell expressing a modified TCR complex and/or a subject system can induce apoptosis in target cells displaying target epitopes on their surface. In some embodiments, cytotoxicity can be determined in vitro or in vivo. In some embodiments, determining cytotoxicity can comprise determining a level of disease after administration of cells comprising a modified TCR complex and/or a system provided herein as compared to a level of disease prior to the administration. In some embodiments, determining cytotoxicity can comprise determining a level of disease after administration of cells comprising a modified TCR complex and/or a system provided herein and a level of disease after administration of comparable immune cells lacking the modified TCR complex and/or the system, comparable immune cells lacking one or more components of the modified TCR complex and/or the system (e.g., CAR, modified TCR complex), and/or comparable immune cells in which only one of the first and second antigen binding domains is bound to their respective epitopes. In some embodiments, a level of disease on a target lesion can be measured as a Complete Response (CR); Disappearance of target lesions, Partial Response (PR); at least a 30% decrease in the sum of the longest diameter (LD) of target lesions taking as reference the baseline sum LD, Progression (PD); at least a 20% increase in the sum of LD of target lesions taking as reference the smallest sum LD recorded since the treatment started or the appearance of one or more new lesions, Stable Disease (SD); or, neither sufficient shrinkage to qualify for PR nor sufficient increase to qualify for PD taking as references the smallest sum LD. In some embodiments, a non-target lesion can be measured. A level of disease of a non-target lesion can be Complete Response (CR); disappearance of all non-target lesions and normalization of tumor marker level, Non-Complete Response; persistence of one or more non-target lesions, Progression (PD); or appearance of one or more new lesions.

In some embodiments, immune cell activity is proliferation of the immune cell. Proliferation of the immune cell can refer to expansion of the immune cell. Proliferation of the immune cell can refer to phenotypic changes of the immune cell. Proliferation of an immune cell comprising a modified TCR complex and/or a system provided herein can be greater than that of a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. Proliferation of an immune cell comprising a modified TCR complex and/or a system provided herein can be about 5 fold to about 10 fold, about 10 fold to about 20 fold, about 20 fold to about 30 fold, about 30 fold to about 40 fold, about 40 fold to about 50 fold, about 50 fold to about 60 fold, about 60 fold to about 70 fold, about 70 fold to about 80 fold, about 80 fold to about 90 fold, about 90 fold to about 100 fold, about 100 fold to about 200 fold, from about 200 fold to about 300 fold, from about 300 fold to about 400 fold, from about 400 fold to about 500 fold, from about 500 fold to about 600 fold, from about 600 fold to about 700 fold greater than the proliferation of a comparable immune cell lacking the modified TCR complex and/or the system provided herein, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, proliferation can comprise quantifying the number of immune cells. Quantifying a number of immune cells can comprise flow cytometry, Trypan Blue exclusion, and/or hemocytometry. Proliferation can also be determined by phenotypic analysis of the immune cells. For example, clumping of immune cells in culture can signify proliferation of immune cells as compared to comparable immune cells lacking the modified TCR complex and/or the system.

In some embodiments, immune cell activity can be differentiation, dedifferentiation, or transdifferentiation. Differentiation, dedifferentiation, or transdifferentation of an immune cell can be determined by evaluating phenotypic expression of markers of differentiation, dedifferentiation, or transdifferentation on a cell surface by flow cytometry. In some embodiments, an immune cell comprising a modified TCR complex and/or a system provided herein has increased differentiation ability as compared to a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, an immune cell comprising a modified TCR complex and/or a system provided herein has increased dedifferentiation ability as compared to a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, an immune cell comprising a modified TCR complex and/or a system provided herein has greater transdifferentiation ability as compared to a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes.

In some embodiments, immune cell activity can be movement and/or trafficking of the immune cell comprising the modified TCR complex and/or the system. In some embodiments, movement can be determined by quantifying localization of the immune cell to a target site. For example, immune cells comprising a modified TCR complex and/or a subject system can be quantified at a target site after administration, for example at a site that is not the target site. Quantification can be performed by isolating a lesion and quantifying a number of immune cells, for example tumor infiltrating lymphocytes, comprising the modified TCR complex and/or the system. Movement and/or trafficking of an immune cell comprising a modified TCR complex and/or a system provided herein can be greater than that of a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, the number of immune cells comprising the modified TCR complex and/or the system at a target site, for example a tumor lesion, can be about 5×, 10×, 15×, 20×, 25×, 30×, 35×, or 40× that of the number of comparable immune cells lacking the modified TCR complex and/or the system, comparable immune cells lacking one or more components of the modified TCR complex and/or the system (e.g., CAR, modified TCR complex), and/or comparable immune cells in which only one of the first and second antigen binding domains is bound to their respective epitopes. Trafficking can also be determined in vitro utilizing a transwell migration assay. In some embodiments, the number of immune cells comprising the modified TCR complex and/or the system at a target site, for example in a transwell migration assay, can be about 5×, 10×, 15×, 20×, 25×, 30×, 35×, or 40× that of the number of comparable immune cells lacking the modified TCR complex and/or the system, comparable immune cells lacking one or more components of the modified TCR complex and/or the system (e.g., CAR, modified TCR complex), and/or comparable immune cells in which only one of the first and second antigen binding domains is bound to their respective epitopes.

In some embodiments, immune cell activity can be exhaustion and/or activation of the immune cell. Exhaustion and/or activation of an immune cell can be determined by phenotypic analysis by flow cytometry or microscopic analysis. For example, expression levels of markers of exhaustion, for instance programmed cell death protein 1 (PD1), lymphocyte activation gene 3 protein (LAG3), 2B4, CD160, Tim3, and T cell immunoreceptor with immunoglobulin and ITIM domains (TIGIT), can be determined quantitatively and/or qualitatively. In some cases, immune cells, such as T cells, can lose effector functions in a hierarchical manner and become exhausted. As a result of exhaustion, functions such as TL-2 production and cytokine expression, as well as high proliferative capacity, can be lost. Exhaustion can also be followed by defects in the production of IFNγ, TNFα and chemokines, as well as in degranulation. Exhaustion or activation of an immune cell comprising a modified TCR complex and/or a system provided herein can be greater than that of a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, the immune cell comprising the modified TCR complex and/or the system provided herein can undergo at least about a 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 20 fold, 30 fold, 40 fold, 50 fold, 60 fold, 70 fold, 80 fold, 90 fold, 100 fold, 150 fold, 200 fold, 250 fold, or over 300 increase in exhaustion or activation as compared to a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes. In some embodiments, the immune cell comprising the modified TCR complex and/or the system provided herein can undergo at least about a 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 20 fold, 30 fold, 40 fold, 50 fold, 60 fold, 70 fold, 80 fold, 90 fold, 100 fold, 150 fold, 200 fold, 250 fold, or over 300 decrease in exhaustion or activation as compared to a comparable immune cell lacking the modified TCR complex and/or the system, a comparable immune cell lacking one or more components of the modified TCR complex and/or the system (e.g., CAR, modified TCR complex), and/or a comparable immune cell in which only one of the first and second antigen binding domains is bound to their respective epitopes.

In some embodiments, binding of the first antigen binding domain to the first epitope and binding of the second antigen domain to the second epitope activates cytotoxicity of a subject immune cell expressing the system. Cytotoxicity can be enhanced as compared to (i) binding of the first antigen binding domain to the first epitope alone, or (ii) binding of the second antigen binding domain to the second epitope alone. Cytotoxicity can be enhanced, as measured by percent killing in a cytotoxicity assay, as compared to (i) binding of the first antigen binding domain to the first epitope alone, or (ii) binding of the second antigen binding domain to the second epitope alone. A percent killing can be from about 5%, 10%, 15%, 20%, 25%, 30%, 35%, 40%, 45%, 50%, 55%, 60%, 65%, 70%, 75%, 80%, 85%, 90%, 95%, or up to about 100% of target cells after contacting as compared to (i) binding of the first antigen binding domain to the first epitope alone, or (ii) binding of the second antigen binding domain to the second epitope alone.

In some embodiments, binding of the first antigen binding domain to the first epitope and binding of the second antigen binding domain to the second epitope activates cytotoxicity of an immune cell expressing the system and reduces a side effect associated with the cytotoxicity as compared to (i) binding of the first antigen binding domain to the first epitope alone, or (ii) binding of the second antigen binding domain to the second epitope alone. In some embodiments, the side effect associated with the cytotoxicity is cytokine release syndrome. A reduction of a side effect, such as a decrease in cytokine release syndrome, can be from about 5%, 10%, 15%, 20%, 25%, 30%, 35%, 40%, 45%, 50%, 55%, 60%, 65%, 70%, 75%, 80%, 85%, 90%, 95%, or up to about 100% reduction as compared to (i) binding of the first antigen binding domain to the first epitope alone, or (ii) binding of the second antigen binding domain to the second epitope alone.

In some embodiments, binding of the first antigen binding domain to the first epitope and binding of the second antigen binding domain to the second epitope activates cytotoxicity of an immune cell expressing the system and increases persistence of cytotoxicity as compared to binding of the first antigen binding domain to the first epitope alone, or binding of the second antigen binding domain to the second epitope alone. Binding of the first antigen binding domain to the first epitope and binding of the second antigen binding domain to the second epitope can activate cytotoxicity of an immune cell expressing the system and increases persistence of said cytotoxicity as compared to binding of the first antigen binding domain to the first epitope alone, or binding of the second antigen binding domain to the second epitope alone when said system is expressed in an immune cell in a subject. An increase in persistence can be determined by quantifying a level of immune cells comprising the system after an administration. An increase in persistence can refer to the presence of immune cells comprising a system provided herein from 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 35 days, 40 days, 2 months, 3 months, 4 months, 5 months, 6 months, 7 months, 8 months, 9 months, 10 months, 1 year or more after administering as compared to comparable immune cells lacking the system, comparable immune cells lacking one or more components of the system (e.g., CAR, modified TCR complex), and/or a comparable immune cell in which only one of the first and second antigen binding domains are bound to their respective epitopes.

In an aspect, the present disclosure provides an isolated host cell expressing any modified TCR complex and/or system of the various embodiments herein (e.g., CAR, modified TCR complex). The isolated host cell can comprise a population of host cells. A host cell can be any suitable cell for expressing a modified TCR complex and/or a subject system. In some cases, the host cell is an immune cell. The immune cell can be a lymphocyte such as a T cell. Non-limiting examples of T cells include CD8+ T cells and CD4+ T cells, αβ T cells, γδ T cells, Vγ962 T cells, V61 T cells, Vδ3 T cells and Vδ5 T cells. In some cases, the lymphocyte expressing a modified TCR complex and/or a subject system is a natural killer (NK) cell, effector T cells, memory T cells, cytotoxic T cells, NKT and/or T helper cells. In some cases, the lymphocyte expressing a modified TCR complex and/or a subject system is a KHYG cell such as KHYG-1 cell or a derivative thereof.

In an aspect, the present disclosure provides an antigen-specific immune cell comprising at least two exogenously introduced antigen binding domains, one of which is linked to a T cell receptor (TCR) complex and another that is linked to a chimeric antigen receptor (CAR). The antigen-specific immune cell can bind specifically to a target cell expressing one or more antigens recognized by the at least two exogenously introduced antigen binding domains. The immune cell can be a lymphocyte such as a T cell. Non-limiting examples of T cells include CD8+ T cells and CD4+ T cells, αβ T cells, γδ T cells, Vγ9δ2 T cells, Vδ1 T cells, Vδ3 T cells and Vδ5 T cells. In some cases, the lymphocyte expressing a modified TCR complex and/or a subject system is a natural killer (NK) cell, effector T cells, memory T cells, cytotoxic T cells, NKT and/or T helper cells. In some cases, the lymphocyte expressing a subject system is a KHYG cell such as KHYG-1 cell or a derivative thereof.

In an aspect, the present disclosure provides a population of immune cells, individual immune cells expressing any modified TCR complex and/or system of the various embodiments herein, and wherein the population of immune cells is characterized in that: upon exposing the population of immune cells to a target cell population in a subject, the population of immune cells induces death of the target cells. The population of immune cells can induce death of at least 5%, 10%, 15%, 20%, 25%, 30%, 35%, 40%, 45%, 50%, 55%, 60%, 65%, 70%, 75%, 80%, 85%, 90%, 95%, or up to about 100% of the target cells and within about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 35 days, 40 days, 2 months, 3 months, 4 months, 5 months, 6 months, 7 months, 8 months, 9 months, 10 months, 1 year or more after the exposing.

The population of immune cell can comprise any of a variety of immune cells. In some cases, the population of immune cells comprises lymphocytes. The lymphocytes can be T cells. Non-limiting examples of T cells include CD8+ T cells and CD4+ T cells, αβ T cells, γδ T cells, Vγ9δ2 T cells, Vδ1 T cells, Vδ3 T cells and Vδ5 T cells. In some cases, the lymphocyte is a natural killer (NK) cell, effector T cells, memory T cells, cytotoxic T cells, NKT and/or T helper cells. In some embodiments, the lymphocyte expressing a modified TCR complex and/or a subject system is a KHYG cell such as KHYG-1 cell or a derivative thereof.

The population of immune cells can comprise any suitable number of cells. The number of immune cells can be determined as the number of cells used in an in vitro assay. The number of immune cells can be determined as the number of cells administered to a subject. The number of immune cells can be determined as the number of cells prior to activation of any immune cell activity, such as proliferation and/or expansion. The population of immune cells can comprise at least about 1×10⁶ cells, at least about 2×10⁶ cells, at least about 3×10⁶ cells, at least about 4×10⁶ cells, at least about 5×10⁶ cells, at least about 6×10⁶ cells, at least about 7×10⁶ cells, at least about 8×10⁶ cells, at least about 9×10⁶ cells, 1×10⁷ cells, at least about 2×10⁷ cells, at least about 3×10⁷ cells, at least about 4×10⁷ cells, at least about 5×10⁷ cells, at least about 6×10⁷ cells, at least about 7×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 7×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 7×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 7×10¹⁰ cells, at least about 8×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 6×10¹¹ cells, at least about 7×10¹¹ cells, at least about 8×10¹¹ cells, at least about 9×10¹¹ cells, or at least about 1×10¹² cells are administered to a subject. In some embodiments, the population of immune cells can comprise at most about 5×10¹⁰ cells, at most about 4×10¹⁰ cells, at most about 3×10¹⁰ cells, at most about 2×10¹⁰ cells, at most about 1×10¹⁰ cells, at most about 9×10⁹ cells, at most about 8×10⁹ cells, at most about 7×10⁹ cells, at most about 6×10⁹ cells, at most about 5×10⁹ cells, at most about 4×10⁹ cells, at most about 3×10⁹ cells, at most about 2×10⁹ cells, at most about 1×10⁹ cells, at most about 9×10⁸ cells, at most about 8×10⁸ cells, at most about 7×10⁸ cells, at most about 6×10⁸ cells, at most about 5×10⁸ cells, at most about 4×10⁸ cells, at most about 3×10⁸ cells, at most about 2×10⁸ cells, at most about 1×10⁸ cells, at most about 9×10⁷ cells, at most about 8×10⁷ cells, at most about 7×10⁷ cells, at most about 6×10⁷ cells, at most about 5×10⁷ cells, at most about 4×10⁷ cells, at most about 3×10⁷ cells, at most about 2×10⁷ cells, at most about 1×10⁷ cells, at most about 9×10⁶ cells, at most about 8×10⁶ cells, at most about 7×10⁶ cells, at most about 6×10⁶ cells, at most about 5×10⁶ cells, at most about 4×10⁶ cells, at most about 3×10⁶ cells, at most about 2×10⁶ cells, at most about 1×10⁶ cells, at most about 9×10⁵ cells, at most about 8×10⁵ cells, at most about 7×10⁵ cells, at most about 6×10⁵ cells, at most about 5×10⁵ cells, at most about 4×10⁵ cells, at most about 3×10⁵ cells, at most about 2×10⁵ cells, or at most about 1×10⁵ cells. The population of immune cells can be administered to a subject in need thereof. For example, about 5×10¹⁰ cells may be administered to a subject. In some cases, a population of cells can be expanded to sufficient numbers for therapy. For example, 5×10⁷ cells can undergo rapid expansion to generate sufficient numbers for therapeutic use. Any number of cells can be administered to a subject, for example by infusion, for therapeutic use. A patient may be infused, for example, with a number of cells between about 1×10⁶ to 5×10¹², inclusive. A patient may be infused with as many cells that can be generated for them.

In any of the cells of the various aspects herein, the cell may exhibit specific binding to two antigens simultaneously present in a target cell. The antigen may be present on the target cell surface or, in some cases, can be an intracellular protein of a target cell that is displayed by another cell, such as in the context of MHC.

In various embodiments of the aspects herein, the antigen binding domain linked to the CAR may primarily mediate interaction between the immune cell and the target cell and the antigen binding domain linked to the modified TCR complex may primarily mediate an immune cell activity when the interaction between the immune cell and the target cell takes place. Immune cell activity, as previously described herein, can include clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell.

In an aspect, provided herein is a method of inducing activity of an immune cell and/or a target cell, comprising (a) expressing a modified TCR complex and/or a system disclosed herein in an immune cell; and (b) contacting a target cell with the immune cell under conditions that induce activity of the immune cell and/or the target cell. In some embodiments, the system expressed in the immune cell comprises a modified T cell receptor (TCR) complex comprising two or more antigen binding domains, optionally in tandem, linked to (i) at least one TCR chain selected from an alpha chain, a beta chain, a gamma chain and a delta chain of a TCR; (ii) an epsilon chain, a delta chain, and/or a gamma chain of a cluster of differentiation 3 (CD3); or (iii) a CD3 zeta chain. In some embodiments, the system expressed in the immune cell comprises a chimeric antigen receptor (CAR) comprising a first antigen binding domain having binding specificity for a first epitope, a transmembrane domain, and an intracellular signaling domain; and a modified T cell receptor (TCR) complex comprising a second antigen binding domain linked to at least one of (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain.

Upon contacting the target cell with the immune cell expressing the system, the first antigen binding domain and/or the second antigen binding domain may bind to their respective epitopes. These epitopes, for example, are present on the target cell. The binding of the first antigen binding domain and/or the second antigen binding domain to their respective epitopes can activate cytotoxicity of the immune cell. In some cases, the cytotoxicity activated in the immune cell when both the first antigen binding domain and the second antigen binding domain is enhanced as compared to a comparable immune cell lacking the system, a comparable immune cell lacking one or more components of the system (e.g., CAR, modified TCR complex), and/or a comparable immune cell expressing the system and wherein only one of the first antigen binding domain and the second antigen binding domain is bound to the respective epitope. The binding of the first antigen binding domain and/or binding of the second antigen binding domain to their respective epitopes can activate cytotoxicity of the immune cell and reduce a side effect associated with the cytotoxicity. In some cases, the reduction in the side effect associated with cytotoxicity is greater as compared to a comparable immune cell lacking the system, a comparable immune cell lacking one or more components of the system (e.g., CAR, modified TCR complex), and/or a comparable immune cell expressing the system and wherein only one of the first antigen binding domain and the second antigen binding domain is bound to the respective epitope. In some cases, the side effect which is reduced is cytokine release syndrome. The binding of the first antigen binding domain and/or binding of the second antigen binding domain to their respective epitopes can activate cytotoxicity of the immune cell and increase persistence of the cytotoxicity. In some cases, the persistence of cytotoxicity is increased as compared to a comparable immune cell lacking the system, a comparable immune cell lacking one or more components of the system (e.g., CAR, modified TCR complex), and/or a comparable immune cell expressing the system and wherein only one of the first antigen binding domain and the second antigen binding domain is bound to the respective epitope. In some cases, cytotoxicity of the immune cell induces death of a target cell.

In various embodiments of a method of inducing activity of the immune cell and/or target cell, the immune cell can be any of a variety of immune cells. In some cases, the immune cell comprises a lymphocyte. The lymphocyte can be T cell. Non-limiting examples of T cells include CD8+ T cells and CD4+ T cells, αβ T cells, γδ T cells, Vγ9δ2 T cells, Vδ1 T cells, Vδ3 T cells and Vδ5 T cells. In some cases, the lymphocyte is a natural killer (NK) cell, effector T cells, memory T cells, cytotoxic T cells, NKT and/or T helper cells. In some cases, the lymphocyte expressing a modified TCR complex and/or a subject system is a KHYG cell such as KHYG-1 cell or a derivative thereof.

In various embodiments of a method of inducing activity of the immune cell and/or target cell, the target cell can be any of a variety of cell types. The target cell can be, for example, a cancer cell, a hematopoietic cell, or a solid tumor cell. The target cell can, in some cases, be a cell identified in one or more of heart, blood vessels, salivary gland, esophagus, stomach, liver, gallbladder, pancreas, intestine, colon, rectum, anus, endocrine gland, adrenal gland, kidney, ureter, bladder, lymph node, tonsils, adenoid, thymus, spleen, skin, muscle, brain, spinal cord, nerve, ovary, fallopian tube, uterus, vagina, mammary gland, testes, prostate, penis, pharynx, larynx, trachea, bronchi, lung, diaphragm, cartilage, ligaments, and tendon. The target cell can be a diseased cell.

In an aspect, the present disclosure provides a method of treating a cancer of a subject. In some embodiments, the method comprises administering to a subject an antigen-specific immune cell comprising a modified TCR complex or a system disclosed herein. In some embodiments, the antigen-specific immune cell comprises a modified T cell receptor (TCR) complex comprising two or more antigen binding domains, optionally in tandem, linked to (i) at least one TCR chain selected from an alpha chain, a beta chain, a gamma chain and a delta chain of a TCR; (ii) an epsilon chain, a delta chain, and/or a gamma chain of a cluster of differentiation 3 (CD3); or (iii) a CD3 zeta chain. In some embodiments, the antigen-specific immune cell comprises a chimeric antigen receptor (CAR) comprising a first antigen binding domain and a modified T cell receptor (TCR) complex comprising a second antigen binding domain. In some embodiments, the method comprises (a) administering to a subject an antigen-specific immune cell comprising a chimeric antigen receptor (CAR) comprising a first antigen binding domain and a modified T cell receptor (TCR) complex comprising a second antigen binding domain, wherein a target cell of a cancer of the subject expresses one or more antigens recognized by the first and/or second antigen binding domain, and wherein the immune cell binds specifically to the target cell, and (b) contacting the target cell with the antigen-specific immune cell via the first and/or second antigen binding domains under conditions that induces an immune cell activity of the immune cell against the target cell, thereby inducing death of the target cell of the cancer.

In an aspect, the present disclosure provides a method of treating a cancer of a subject, comprising (a) administering to a subject an antigen-specific immune cell, wherein the antigen-specific immune cell is a genetically modified immune cell expressing any modified TCR complex and/or system of the embodiments provided herein; and (b) contacting the target cell with the antigen-specific immune cell under conditions that induces an immune cell activity of the immune cell against a target cell of a cancer of the subject, thereby inducing death of the target cell of the cancer.

In some embodiments, a method of treating a cancer of a subject comprises genetically modifying an immune cell to yield the antigen-specific immune cell.

Upon contacting the target cell with the antigen-specific immune cell, immune cell activity against a target cell of a cancer of the subject can induce death of the target cell. An immune cell activity can be selected from the group consisting of: clonal expansion of the immune cell; cytokine release by the immune cell; cytotoxicity of the immune cell; proliferation of the immune cell; differentiation, dedifferentiation or transdifferentiation of the immune cell; movement and/or trafficking of the immune cell; exhaustion and/or reactivation of the immune cell; and release of other intercellular molecules, metabolites, chemical compounds, or combinations thereof by the immune cell. In some cases, the immune cell activity is cytotoxicity of the immune cell. Cytotoxicity of an immune cell against a target cell can yield at least about 5%, 10%, 15%, 20%, 25%, 30%, 35%, 40%, 45%, 50%, 55%, 60%, 65%, 70%, 75%, 80%, 85%, 90%, 95%, or up to about 100% reduction in a cancer of a subject. In some embodiments, an immune cell activity can be cytokine release by an immune cell. In some cases, cytokine is released by the immune cell. The amount of cytokine released by the immune cell can be at least 3%, 5%, 10%, 15%, 20%, 25%, 30%, 35%, 40%, 45%, 50%, 55%, 60%, 65%, 70%, 75%, 80%, 85%, 90%, 95%, or up to about 100% less than that of comparable immune cell lacking the modified TCR complex and/or the system, a comparable immune cell lacking one or more components of the modified TCR complex and/or the system (e.g., CAR, modified TCR), and/or a comparable immune cell in which only one of the first and second antigen binding domains is bound to their respective epitopes. In some cases, persistence of the immune cell activity is greater when both the first and second antigen binding domain bind their respective epitopes, as compared to binding of only the first antigen binding domain alone, or binding of the second antigen binding domain alone.

In various embodiments of a method of treating a cancer of a subject, the immune cell can be any of a variety of immune cells. In some cases, the immune cell comprises a lymphocyte. The lymphocyte can be T cell. Non-limiting examples of T cells include CD8+ T cells and CD4+ T cells. In some cases, the lymphocyte is a natural killer (NK) cell. In some cases, the lymphocyte expressing a modified TCR complex and/or a subject system is a KHYG cell such as KHYG-1 cell or a derivative thereof.

In various embodiments of a method of treating a cancer of a subject, the cancer can be any one of a variety of cancers. The cancer is, for example, bladder cancer, bone cancer, brain cancer, breast cancer, cervical cancer, colon cancer, esophageal cancer, gastric cancer, glioma, head and neck cancer, kidney cancer, leukemia, acute myeloid leukemia (AML), multiple myeloma, liver cancer, lung cancer, lymphoma, melanoma, mesothelioma, medulloblastoma, ovarian cancer, pancreatic cancer, prostate cancer, rectal cancer, skin cancer, testicular cancer, tracheal cancer, or vulvar cancer. In an aspect, the present disclosure provides a composition. In some embodiments, the composition comprises a modified T cell receptor (TCR) complex and/or a system disclosed herein. In some embodiments, the composition comprises a modified T cell receptor (TCR) complex comprising two or more binding domain which exhibit specific binding to two or more epitopes, wherein said antigen binding domains are, optionally in tandem, linked to (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain. In some embodiments, the composition comprises one or more polynucleotides that encodes (a) a chimeric antigen receptor (CAR) comprising a first antigen binding domain having binding specificity for a first epitope, a transmembrane domain, and an intracellular signaling domain; and (b) a modified T cell receptor (TCR) complex comprising a second antigen binding domain which exhibits specific binding to a second epitope, wherein said second antigen binding domain is linked to: at least one TCR chain selected from an alpha chain, a beta chain, a gamma chain and a delta chain of a T cell receptor; an epsilon chain, a delta chain, and/or a gamma chain of cluster of differentiation 3 (CD3); or a CD3 zeta chain. The composition can comprise one or more one or more polynucleotides that encodes (a) a chimeric antigen receptor (CAR) comprising a first antigen binding domain having binding specificity for a first epitope, a transmembrane domain, and an intracellular signaling domain; and (b) a second antigen binding domain linked to: an alpha chain, a beta chain, a gamma chain, and/or a delta chain of a T cell receptor; an epsilon chain, a delta chain, and/or a gamma chain of cluster of differentiation 3 (CD3); or a CD3 zeta chain. In some embodiments, one or more polynucleotides comprises a promoter operably linked thereto. The one or more polynucleotides can comprise deoxyribonucleic acid (DNA) and/or ribonucleic acid (RNA). In some embodiments, one or more of the components of the modified T cell receptor (TCR) complex or system encoded by the one or more polynucleotides is joined by a linker that separates two or more nucleic acid coding regions. A linker can be a 2A sequence, a furin-V5-SGSGF2A, and the like.

In an aspect, the present disclosure provides a method of producing a modified immune cell, comprising genetically modifying the immune cell by expressing a composition provided herein in the immune cell, thereby producing said modified immune cell.

In various embodiments of the aspects herein, immune cells comprising a modified TCR complex and/or a system provided herein can be used to induce death of a target cell. A variety of target cells can be killed using the modified TCR complex and/or the systems, and methods of the disclosure. A target cell to which this method can be applied includes a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can be in a living organism. A target cell can be a genetically modified cell. A

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and/or an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested, for example in in vitro experiments, from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

A target call can be identified in one or more of heart, blood vessels, salivary gland, esophagus, stomach, liver, gallbladder, pancreas, intestine, colon, rectum, anus, endocrine gland, adrenal gland, kidney, ureter, bladder, lymph node, tonsils, adenoid, thymus, spleen, skin, muscle, brain, spinal cord, nerve, ovary, fallopian tube, uterus, vagina, mammary gland, testes, prostate, penis, pharynx, larynx, trachea, bronchi, lung, diaphragm, cartilage, ligaments, and tendon.

Non-limiting examples of cells which can be target cells include, but are not limited to, hematopoietic cells, lymphoid cells, such as B cell, T cell (Cytotoxic T cell, Natural Killer T cell, Regulatory T cell, T helper cell), Tumor infiltrating lymphocyte (TIL), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

Of particular interest are cancer cells. In some embodiments, the target cell is a cancer cell. A cancer can be a solid tumor or a hematological tumor. A cancer can be metastatic. A cancer can be a relapsed cancer. Non-limiting examples of cancer cells include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The first and/or second antigen binding domains can bind to epitopes present on antigens of cancer cells.

In some embodiments, the target cells can form a tumor. A tumor treated with the methods herein can result in stabilized tumor growth (e.g., one or more tumors do not increase more than 1%, 5%, 10%, 15%, or 20% in size, and/or do not metastasize). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more as a result of treatment according to methods provided herein. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

Death of target cells can be determined by any suitable method, including, but not limited to, counting cells before and after treatment, or measuring the level of a marker associated with live or dead cells (e.g. live or dead target cells).

Degree of cell death can be determined by any suitable method. In some embodiments, degree of cell death is determined with respect to a starting condition. For example, an individual can have a known starting amount of target cells, such as a starting cell mass of known size or circulating target cells at a known concentration. In such cases, degree of cell death can be expressed as a ratio of surviving cells after treatment to the starting cell population. In some embodiments, degree of cell death can be determined by a suitable cell death assay. A variety of cell death assays are available, and can utilize a variety of detection methodologies. Examples of detection methodologies include, without limitation, the use of cell staining, microscopy, flow cytometry, cell sorting, and combinations of these.

When a tumor is subject to surgical resection following completion of a therapeutic period, the efficacy of treatment in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%). In some embodiments, the necrosis percentage of the resected tissue is 100%, that is, no living tumor tissue is present or detectable.

In various embodiments of the aspects provided herein, exposing a target cell to or contacting a target cell with an immune cell or population of immune cells can be conducted either in vitro or in vivo. Exposing a target cell to an immune cell or population of immune cells generally refers to bringing the target cell in contact with the immune cell and/or in sufficient proximity such that an antigen (e.g., comprising an epitope) of a target cell (e.g., membrane bound or non-membrane bound) can bind to the antigen binding domain of the first antigen binding domain and/or the second antigen binding domain. Exposing a target cell to an immune cell or population of immune cells in vitro can be accomplished by co-culturing the target cells and the immune cells. Target cells and immune cells can be co-cultured, for example, as adherent cells or alternatively in suspension. Target cells and immune cells can be co-cultured in various suitable types of cell culture media, for example with supplements, growth factors, ions, etc. Exposing a target cell to an immune cell or population of immune cells in vivo can be accomplished, in some cases, by administering the immune cells to a subject, for example a human subject, and allowing the immune cells to localize to the target cell via the circulatory system. In some cases, an immune cell can be delivered to the immediate area where a target cell is localized, for example, by direct injection.

Exposing or contacting can be performed for any suitable length of time, for example at least 1 minute, at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, at least 12 hours, at least 16 hours, at least 20 hours, at least 24 hours, at least 2 days, at least 3 days, at least 4 days, at least 5 days, at least 6 days, at least 1 week, at least 2 weeks, at least 3 weeks, at least 1 month or longer.

In various embodiments of the aspects herein, a modified TCR complex and/or a system provided herein is expressed in a host cell (e.g., an immune cell, e.g., an antigen-specific immune cell). The host cell can be a human cell. The host cell can be a non-human cell. A host cell can be autologous or allogeneic to a subject in need thereof. In some cases, a host cell can be xenogeneic. A host cell can be an immune cell such as a lymphocyte or myeloid cell. A host cell can be a T cell, B cell, NK cell, and the like. In some embodiments, the host cell can be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin− cell, a CD25+, a CD25− cell, a CD27+, a CD27-cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor G1 positive cell, a lectin-like receptor G1 negative cell, or an differentiated or de-differentiated cell thereof. In some embodiments, the host cell may be positive for two or more factors. For example, the host cell may be CD4+ and CD8+. In some embodiments, the host cell may be negative for two or more factors. For example, the host cell may be CD25−, CD44−, and CD69−. In some embodiments, the host cell may be positive for one or more factors, and negative for one or more factors. For example, the cell may be CD4+ and CD8−. In some embodiments, host cells may be selected for having or not having one or more given factors (e.g., cells may be separated based on the presence or absence of one or more markers described herein).

In some embodiments, host cells that are selected may also be expanded in vitro Selected and/or expanded host cells may be administered to a subject in need thereof. It should be understood that cells used in any of the methods disclosed herein may be a mixture (e.g., two or more different cells) of any of the cells disclosed herein. For example, a composition may comprise a mixture of different cells, for example T cells and B cells. The mixture can include, for example, a stem memory T_{SCM} cell comprising CD45RO (−), CCR7(+), CD45RA (+), CD62L+ (L-selectin), CD27+, CD28+ and IL-7Rα+, stem memory cells can also express CD95, IL-2Rβ, CXCR3, and LFA-1, and show numerous functional attributes distinctive of stem memory cells. The mixture can include, for example, central memory T_{CM} cells comprising L-selectin and CCR7, where the central memory cells can secrete, for example, IL-2, but not IFNγ or IL-4. The mixture can include, for example, effector memory T_{EM} cells comprising L-selectin or CCR7 and produce, for example, effector cytokines such as IFNγ and IL-4.

A host cell can be obtained from a subject. In some cases, a host cell can be a population of T cells, NK cell, B cells, and the like obtained from a subject. T cells can be obtained from a number of sources, including PBMCs, bone marrow, lymph node tissue, cord blood, thymus tissue, and tissue from a site of infection, ascites, pleural effusion, spleen tissue, and tumors. In some embodiments, T cells can be obtained from a unit of blood collected from a subject using any number of techniques, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps.

In some embodiments, a population of immune cells provided herein can be heterogeneous. In some embodiments, cells used can be composed of a heterogeneous mixture of CD4 and CD8 T cells. Said CD4 and CD8 cells can have phenotypic characteristics of circulating effector T cells. Said CD4 and CD8 cells can also have a phenotypic characteristic of effector-memory cells. In some embodiment, cells can be central-memory cells.

In some embodiments, host cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem cell or a non-pluripotent stem cell. In some cases, the cell can be any immune cell, including any T-cell such as tumor infiltrating cells (TILs), such as CD3+ T-cells, CD4+ T-cells, CD8+ T-cells, or any other type of T-cell. The T cell can also include memory T cells, memory stem T cells, or effector T cells. The T cells can also be selected from a bulk population, for example, selecting T cells from whole blood. The T cells can also be expanded from a bulk population. The T cells can also be skewed towards particular populations and phenotypes. For example, the T cells can be skewed to phenotypically comprise, CD45RO (−), CCR7 (+), CD45RA (+), CD62L (+), CD27 (+), CD28 (+) and/or IL-7Rα (+). Suitable cells can be selected that comprise one of more markers selected from a list comprising: CD45RO (−), CCR7 (+), CD45RA (+), CD62L (+), CD27 (+), CD28 (+) and/or IL-7Rα (+). Host cells also include stem cells such as, by way of example, embryonic stem cells, induced pluripotent stem cells, hematopoietic stem cells, neuronal stem cells and mesenchymal stem cells. Host cells can comprise any number of primary cells, such as human cells, non-human cells, and/or mouse cells. Host cells can be progenitor cells. Host cells can be derived from the subject to be treated (e.g., patient). Host cells can be derived from a human donor. Host cells can be stem memory TSCM cells comprised of CD45RO (−), CCR7(+), CD45RA (+), CD62L+(L-selectin), CD27+, CD28+ and IL-7Rα+, said stem memory cells can also express CD95, IL-2Rβ, CXCR3, and LFA-1, and show numerous functional attributes distinctive of said stem memory cells. Host cells can be central memory TCM cells comprising L-selectin and CCR7, said central memory cells can secrete, for example, IL-2, but not IFNγ or IL-4. Host cells can also be effector memory TEM cells comprising L-selectin or CCR7 and produce, for example, effector cytokines such as IFNγ and IL-4.

A number of viral based systems have been developed for gene transfer into mammalian cells. For example, retroviruses, lentiviruses, and adenoviruses provide a convenient platform for gene delivery systems. A subject system can be inserted into a vector and packaged in retroviral particles using techniques known in the art. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells. They also have the added advantage of low immunogenicity.

In an aspect, a nucleic acid encoding a system comprising a modified TCR complex and/or CAR can be delivered virally or non-virally. Viral delivery systems (e.g., viruses comprising the pharmaceutical compositions of the disclosure) can be administered by direct injection, stereotaxic injection, intracerebroventricularly, by minipump infusion systems, by convection, catheters, intravenous, parenteral, intraperitoneal, and/or subcutaneous injection, to a cell, tissue, or organ of a subject in need. In some instances, cells can be transduced in vitro or ex vivo with viral delivery systems. The transduced cells can be administered to a subject having a disease. For example, a stem cell can be transduced with a viral delivery system comprising a pharmaceutical composition and the stem cell can be implanted in the patient to treat a disease. In some instances, the dose of transduced cells given to a subject can be about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single dose.

A packaging cell line can be used to generate viral particles comprising a modified TCR complex and/or a subject system provided herein. A packaging cell line can also be utilized to perform methods provided herein. Packaging cells that can be used include, but are not limited to, HEK 293 cells, HeLa cells, and Vero cells to name a few. In some cases, supernatant of the packaging cell line is treated by PEG precipitation for concentrating viral particles. In other cases, a centrifugation step can be used to concentrate viral particles. For example a column can be used to concentration a virus during a centrifugation. In some cases, a precipitation occurs at no more than about 4° C. (for example about 3° C., about 2° C., about 1° C., or about 1° C.) for at least about 2 hours, at least about 3 hours, at least about 4 hours, at least about 6 hours, at least about 9 hours, at least about 12 hours, or at least about 24 hours. In some cases, viral particles can be isolated from the PEG-precipitated supernatant by low-speed centrifugation followed by CsCl gradient. The low-speed centrifugation can be about 4000 rpm, about 4500 rpm, about 5000 rpm, or about 6000 rpm for about 20 minutes, about 30 minutes, about 40 minutes, about 50 minutes or about 60 minutes. In some cases, viral particles are isolated from PEG-precipitated supernatant by centrifugation at about 5000 rpm for about 30 minutes followed by CsCl gradient.

A virus (e.g., lentivirus) can be introduced to a subject cell or to a population of subject cells at about, from about, at least about, or at most about 1-3 hrs., 3-6 hrs., 6-9 hrs., 9-12 hrs., 12-15 hrs., 15-18 hrs., 18-21 hrs., 21-23 hrs., 23-26 hrs., 26-29 hrs., 29-31 hrs., 31-33 hrs., 33-35 hrs., 35-37 hrs., 37-39 hrs., 39-41 hrs., 2 days, 3 days, 4 days, 5 days, 6 days, 7 days, 8 days, 9 days, 10 days, 14 days, 16 days, 20 days, or longer than 20 days after a stimulation or activation step, for instance anti-CD3, anti-CD28, or a combination thereof. In some cases, a viral vector encodes for a modified TCR complex and/or a system, for example a CAR-T, modified TCR complex, or a combination thereof. In some cases, a viral vector encodes for a CAR-T. In some cases, a viral vector encodes for a modified TCR complex. An immune cell can be transduced with viral particles encoding for both a CAR and a modified TCR complex. An immune cell can be transduced with viral particles encoding for a CAR. An immune cell can be transduced with viral particles encoding for a modified TCR complex. A nucleic acid encoding a modified TCR complex and/or a subject system can be inserted randomly into the genome of a cell. A nucleic acid encoding a modified TCR complex and/or a system can encode its own promoter or can be inserted into a position where it is under the control of an endogenous promoter of a cell. Alternatively, a nucleic acid encoding a modified TCR complex and/or a system can be inserted into a gene, such as an intron of a gene, an exon of a gene, a promoter, or a non-coding region. Expression of a modified TCR complex and/or a system can be verified by an expression assay, for example, qPCR or by measuring levels of RNA in transduced cells. Expression level can be indicative also of copy number. For example, if expression levels are high, this can indicate that more than one copy of a nucleic acid encoding a modified TCR complex and/or a system was integrated in a genome of a cell. Alternatively, high expression can indicate that a nucleic acid encoding a modified TCR complex and/or a system was integrated in a highly transcribed area, for example, near a highly expressed promoter. Expression can also be verified by measuring protein levels, such as through Western blotting.

Cell viability of a subject cell or subject population of cells can be measured by fluorescence-activated cell sorting (FACS). In some cases, cell viability is measured after a viral or a non-viral vector comprising a nucleic acid encoding a modified TCR complex and/or a subject system is introduced to a cell or to a population of cells. In some cases, at least about, or at most about, or about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.8%, or 100% of the cells in a population of cells are viable after a viral vector is introduced to the cell or to the population of cells. In some cases, cell viability is measured at about, at least about, or at most about 4 hours, 6 hours, 8 hours, 10 hours, 12 hours, 18 hours, 20 hours, 24 hours, 30 hours, 36 hours, 40 hours, 48 hours, 54 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, or longer than 240 hours after a viral vector is introduced to a cell and/or to a population of cells. In some cases, cell viability is measured at about, at least about, or at most about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 45 days, 50 days, 60 days, 70 days, 90 days, or longer than 90 days after a viral vector is introduced to a cell or population of cells. In some cases, cellular toxicity is measured at about, at least about, or at most about 4 hours, 6 hours, 8 hours, 12 hours, 18 hours, 24 hours, 30 hours, 36 hours, 42 hours, 48 hours, 54 hours, 60 hours, 66 hours, 72 hours, 78 hours, 84 hours, 90 hours, 96 hours, 102 hours, 108 hours, 114 hours, 120 hours, 126 hours, 132 hours, 138 hours, 144 hours, 150 hours, 156 hours, 168 hours, 180 hours, 192 hours, 204 hours, 216 hours, 228 hours, 240 hours, or longer than 240 hours after a viral vector is introduced to a cell or to a population of cells.

In some embodiments, one or more nucleic acids encoding a modified TCR complex and/or a system comprising a modified TCR complex and/or CAR can be delivered by viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

In some embodiments, immune cells expressing a modified TCR complex and/or a system provided herein are administered. Immune cells can be administered before, during, or after the occurrence of a disease or condition, and the timing of administering the immune cells can vary. For example, immune cells expressing a modified TCR complex and/or a subject system can be used as a prophylactic and can be administered continuously to subjects with a propensity to conditions or diseases in order to prevent the occurrence of the disease or condition. The immune cells can be administered to a subject during or as soon as possible after the onset of the symptoms. The administration can be initiated within the first 48 hours of the onset of the symptoms, within the first 24 hours of the onset of the symptoms, within the first 6 hours of the onset of the symptoms, or within 3 hours of the onset of the symptoms. The initial administration can be via any suitable route, such as by any route described herein using any formulation described herein. Immune cells can be administered as soon as is practicable after the onset of a disease or condition is detected or suspected, and for a length of time necessary for the treatment of the disease, such as, for example, from about 1 month to about 3 months. The length of treatment can vary for each subject.

The compositions provided herein comprising immune cells expressing the modified TCR complex and/or the subject system may be administered to a subject using known modes and techniques. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection of infusion of the formulations can be used to effect such administration. Formulations comprising the subject compositions can be administered to a subject in an amount that is effective for treating and/or prophylaxis of the specific indication or disease. A physician can determine appropriate dosages to be used. Compositions comprising immune cells expressing a modified TCR complex and/or a subject system may be independently administered 4, 3, 2, or once daily, every other day, every third day, every fourth day, every fifth day, every sixth day, once weekly, every eight days, every nine days, every ten days, bi-weekly, monthly and bi-monthly.

Compositions and methods provided herein can be combined with secondary therapies including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine. Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including α and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; CAR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Immune cells comprising any modified TCR complex and/or system provided herein can be administered to a subject in conjunction with (e.g., before, simultaneously, or following) any number of relevant treatment modalities, including but not limited to treatment with agents such as antiviral therapy, cidofovir and interleukin-2, or Cytarabine (also known as ARA-C). In some cases, the subject immune cells can be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. The engineered cell composition can also be administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, the subject immune cell compositions can be administered following B-cell ablative therapy such as agents that react with CD20, e.g., Rituxan. For example, subjects can undergo standard treatment with high dose chemotherapy followed by peripheral blood stem cell transplantation. In certain embodiments, following the transplant, subjects can receive an infusion of immune cells, e.g., expanded immune cells comprising a modified TCR complex and/or a subject system. Additionally, expanded immune cells can be administered before or following surgery.

In some cases, for example, in the compositions, formulations and methods of treating cancer, the unit dosage of the composition or formulation administered can be 5, 10, 15, 20, 25, 30, 35, 40, 45, 50, 55, 60, 65, 70, 75, 80, 85, 90, 95 or 100 mg. In some cases, the total amount of the composition or formulation administered can be 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 40, 50, 60, 70, 80, 90, or 100 g.

### EXAMPLES

The examples below are intended to be purely exemplary of the invention and should therefore not be considered to limit the invention in any way. The following examples and detailed description are offered by way of illustration and not by way of limitation.

Various aspects of the disclosure are further illustrated by the following non-limiting examples.

### Example 1: Generation of Anti-BCMA sdAbs

### Immunization

Two camels were immunized with recombinant BCMA ECD protein (ACRO Biosystems, Cat. #: BCA-H522y, SEQ ID NO: 1) under all current animal welfare regulations. For immunization, the antigen was formulated as an emulsion with CFA (primary immunization) or IFA (boost immunization). The antigen was administered intramuscularly by double-spot injections at the neck. Each animal received two injections of the emulsion containing 100 μg of BCMA ECD, and 4 subsequent injections containing 50 μg of antigen at weekly intervals. At different time points during immunization, 10 ml blood samples were collected from the animal and sera were prepared. The induction of an antigen specific humoral immune response was verified using the serum samples in an ELISA-based experiment with immobilized BCMA ECD protein. Five days after the last immunization, 150 ml blood sample was collected from each animal. Peripheral blood lymphocytes (PBLs), as the genetic source of the camel heavy chain immunoglobulins (HCAbs), were isolated from the 300 ml blood sample using a Ficoll-Paque gradient (Amersham Biosciences), yielding ˜1×10⁹ PBLs. The maximal diversity of antibodies is expected to be equal to the number of sampled B-lymphocytes, which is about 10% of the number of PBLs (1×10⁸). The fraction of heavy-chain antibodies in camel is up to 20% of the number of B-lymphocytes. Therefore, the maximal diversity of HCAbs in the 300 ml blood sample is estimated to be approximately 2×10⁷ different molecules.

### Library Construction

RNA extracted from PBLs was used as starting material for RT-PCR to amplify sdAb encoding gene fragments. These fragments were cloned into an in-house phagemid vector. In frame with the sdAb coding sequence, the vector also codes a C-terminal (His)₆ tag. The library size is more than 1×10⁹. The library phage was prepared according to a standard protocol and stored after filter sterilization at 4° C. for further use.

### Binder Isolation and High-Throughput Screening

Binders were isolated with the above libraries using solid-phase panning as well as cell-based panning. One round of panning was performed for both conditions. Each selection output was analyzed for the number of total output clones, percentage of BCMA positive clones (by ELISA) and sequence diversity of BCMA-specific binders. Based on these parameters the best panning output was selected for high-throughput screening. To this end, the selected output phage was used to infect exponentially growing E. coli cells. The double-strand DNA pool of the output was extracted, the sdAb insert was cut from the phagemid vector and inserted into a soluble expression vector for high-throughput screening. In frame with the sdAb coding sequence, the vector also codes a C-terminal (His)₆ tag. Colonies were picked and grown in 96 deep well plates containing 1 ml 2YT medium. The expression of sdAbs was induced by adding 1 mM IPTG in the supernatant.

The sdAbs in the supernatant were analyzed for their abilities to bind to BCMA ECD protein by ELISA, and to BCMA stable cell lines by FACS. All binders were sequenced and some were subjected to further characterization including affinity ranking by surface plasmon resonance (SPR) on a BIAcore T200 instrument. The experiment was carried out as follows: The crude sdAbs proteins were captured through an affinity tag onto the sensorchip. The amount of antibody captured was dependent on the concentration of the crude protein in the supernatant. High-concentration (100 nM) of antigen proteins, i.e. His-tagged human BCMA (ACRO Biosystems, Cat. #: BCA-H522y) and Fc-fused Cynomolgus BCMA (ACRO Biosystems, Cat. #: BCA-C5253, SEQ ID NO: 2), were flowed over the sensorchip surface, and allowed to bind the sdAbs. On-rate (kₒₙ) and off-rate (k_{off}) were roughly calculated based on the association and dissociation at the antigen concentration of 100 nM, and used to estimate the equilibrium dissociation constant (K_{D}).

TABLE 9
Estimated binding affinity of anti-BCMA sdAbs by SPR affinity ranking.

| | Human BCMA, His-tagged | | | Cynomolgus BCMA, Fc fusion | | |
|---|---|---|---|---|---|---|
| clone ID | ka (1/M · s) | kd (1/s) | K_{D} (M) | ka (1/M · s) | kd (1/s) | K_{D} (M) |
| BCMA 1 | 2.9E+07 | 2.9E−01 | 1.00E−08 | 6.7E+05 | 4.3E−04 | 6.40E−10 |
| BCMA 5 | 1.2E+07 | 8.1E−03 | 6.60E−10 | — | — | — |
| BCMA 6 | 5.4E+06 | 2.6E−02 | 4.90E−09 | 1.1E+06 | 9.1E−04 | 8.30E−10 |
| BCMA 7 | 2.3E+06 | 1.0E−01 | 4.60E−08 | 1.3E+06 | 5.6E−02 | 4.30E−08 |
| BCMA 8 | 7.8E+06 | 3.7E−01 | 4.80E−08 | 4.4E+10 | 5.6E+02 | 1.30E−08 |
| BCMA 9 | 5.00E+07 | 4.20E−01 | 8.50E−09 | 6.50E+05 | 1.50E−03 | 2.40E−09 |
| BCMA 10 | 2.80E+06 | 1.30E−01 | 4.70E−08 | 7.90E+05 | 4.00E−02 | 5.00E−08 |
| BCMA 11 | 1.7E+05 | 9.0E−03 | 5.30E−08 | 4.5E+05 | 7.0E−03 | 1.60E−08 |
| BCMA 12 | 1.1E+06 | 2.5E−02 | 2.30E−08 | 9.4E+05 | 4.0E−03 | 4.30E−09 |
| BCMA 13 | 6.9E+05 | 5.3E−02 | 7.70E−08 | 1.4E+06 | 1.8E−02 | 1.30E−08 |

### Example 2: Viral Transfection and Viral Particle Generation

To generate viral particles comprising polynucleic acids encoding any of the systems disclosed herein, lentivirus packaging plasmid mixture including pMDLg/pRRE (Addgene #12251), pRSV-Rev (Addgene #12253), and pMD2.G (Addgene #12259) was properly pre-mixed with a PLVX-EF1A (including target system) vector at a pre-optimized ratio, together with polyetherimide (PEI), and incubated at room temperature for 5 minutes. The transfection mixture was added dropwise to 293-T cells and the then mixed with the cells gently. Transfected 293-T cells were incubated overnight at 37° C. and 5% CO2. 24 hours post transfection, supernatants were collected and centrifuged at 4° C., 500 g for 10 min to remove any cellular debris, followed by an ultracentrifugation step. Centrifuged supernatants were filtered through a 0.45 m PES filter to concentrate the viral supernatants post ultracentrifugation. After centrifugation, the supernatants were carefully discarded and the virus pellets were rinsed with pre-chilled DPBS. The concentration of virus was measured. Virus was aliquoted and stored at −80° C. Viral titer was determined by functional transduction on a T cell line.

Briefly, the lentiviral vector was modified using pLVX-Puro (Clontech #632164) by replacing the original promoter with human elongation factor 1α promoter (hEF1α) and by removing the puromycin resistance gene with EcoRI and BamHI by GenScript. PLVX-EF1A, was further subjected to the lentivirus packaging procedure as described above.

### Example 3: Immune Cell Preparation

Leukocytes were collected in R10 medium, and then mixed with 0.9% NaCl solution at a 1:1 (v/v) ratio. 3 mL lymphoprep medium was added to a 15 mL centrifuge tube. The lymphoprep was slowly layered to form 6 mL diluted lymphocyte mix. The lymphocyte mix was centrifuged at 800 g for 30 minutes without brakes at 20° C. Lymphocyte buffy coat was then collected with a 200 μL pipette. The harvested fraction was diluted at least 6 fold with 0.9% NaCl or R10 to reduce the density of the solution. The harvested fraction was then centrifuged at 250 g for 10 minutes at 20° C. The supernatant was aspirated completely, and 10 mL of R10 was added to the cell pellet. The mixture was further centrifuged at 250 g for 10 minutes at 20° C. The supernatant was then aspirated. 2 mL 37° C. pre-warmed R10 with 100 IU/mL IL-2 was added to the cell pellet, and the cell pellet was re-suspended softly. Cells were quantified and the PBMC sample was ready for experimentation. Human T cells were purified from PBMCs using Miltenyi Pan T cell isolation kit (Cat #130-096-535).

The prepared T cells were subsequently pre-activated for 48 hours with human T cell Activation/Expansion kit (Milteny #130-091-441) by using one loaded anti-Biotin MACSiBead Particle per two cells (bead-to-cell ratio 1:2).

### Example 4: T Cell Transfection

The pre-activated T cells were collected and suspended and re-suspended in 1640 medium containing 300 IU/mL IL-2. A lentiviral vector encoding the system was diluted to MOI=5 with the same medium and infected with 1E+06 activated T cells. The pre-activated T cells were transduced with lentivirus stock in the presence of 8 μg/ml polybrene by centrifugation at 1000 g, 32° C. for 1 h. The transduced cells were then transferred to the cell culture incubator for transgene expression under suitable conditions. The next day, the transduced cells were centrifuged and replaced with fresh media, the cells concentration was measured every 2 days, and fresh media were added to continue the expansion.

### Example 5: Quantification of Receptor Expression

On day 3 and onwards (typically day 3 to day 8) post transduction, cells were evaluated for expression of the system by flow cytometry. An aliquot of cells is collected from the culture, washed, pelleted, and re-suspended in 100 ul PBS, supplemented with 0.5% FBS and diluted binding antibody or antigen protein 1/100. Re-suspended cells are in about 100 ul of Ab. Cells were incubated at 4° C. for 30 minutes. Viability dye eFluor780 or SYTOX Blue viability stain was also added according to manufacturer's instructions. Post incubation, cells were washed twice in PBS and re-suspended in 100 to 200 ul PBS for analysis. The mean fluorescence of the system was quantified by flow cytometry.

For anti-BCMA staining, cells were stained with polyclonal biotin-labeled goat-anti-human BCMA antibodies (R&D, catalog number BAF 193) followed by streptavidin (BD). Flow cytometry analysis for all experiments was performed by using FlowJo (Tree Star, Inc.).

### Example 6: Cytotoxicity Assay

### BCMA Antibody Screening on Epsilon TCR Platform

Anti-BCMA antibody (BCMA1-12) was fused to epsilon-TCR individually for evaluating the cytotoxicity effect with RPMI-8226 cells. On day 3 or 6 post transduction, the effector cells were co-cultured at different effector to target ratios (0.5:1, 1.5:1 and 3:1) at 37° C. for 20 h in 96 well plate. Other wells contained assay buffer only (1640 phenol red free medium plus 2% hiFBS), target cell only (T), effector cell only (E) and max release of target cell (target cells with 1% solution of triton-X 100). Each condition was performed in triplicate, and the cytotoxicity of effector cells was detected by LDH assay kit (Roche). After 20 hr co-culture, the assay plate was centrifuged, and the supernatant was collected and transferred to in a new 96-w plate. The supernatant plate was diluted with an equal volume of LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that effector cells expressing different BCMA sdAb have different cell-killing effects, and antibodies such as BCMA 1, BCMA 2, BCMA 5, BCMA 6, BCMA 8, BCMA 9 and BCMA 12 showed better cell-killing effects, as seen in FIG. 8A, FIG. 8B and FIG. 8C.

IFN-γ expression was assayed by HTRF, as seen in FIG. 8D, FIG. 8E and FIG. 8F. 384-well low volume white plates were used in the assay for IFN-γ detection (human IFNγ kit, Gisbio). The amount of IFN-γ secreted in cytotoxicity assay showed similar trends as the cell-killing effects.

### Multiple Component Systems

Cytotoxicity of anti-BCMA3-epsilon-TCR (BCMA3 eTCR), anti-BCMA2-epsilon-TCR (BCMA2 eTCR), anti-BCMA2-anti-BCMA3-epsilon TCR (tandem BCMA 2&3 eTCR), and anti-BCMA1-anti-BCMA2-anti-BCMA3-gammaTCR (tandem BCMA 1&2&3 gTCR), as well as control untransduced cells was determined in a 20 h co-culture assay, where RPMI-8226 cells (BCMA+) were co-cultured at an effector-to-target cell ratio (E:T) of 0.33:1. Each condition was performed in triplicate, and the cytotoxicity of effector cells was detected by LDH assay kit (Roche). After 20 hr co-culture, the assay plate was centrifuged, and the supernatant was collected and transferred to a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that tandem BCMA antibodies on TCR subunit provided significantly better cell-killing effects than single antibody fused eTCR, suggesting tandem BCMA antibodies on TCR a better choice for cell-killing effect (as shown in FIG. 9).

Cytotoxicity of anti-BCMA1-anti-BCMA2-anti-BCMA3 epsilon-TCR (Tandem BCMA 1-2-3 eTCR), anti-BCMA2-anti-BCMA3 epsilon-TCR (Tandem BCMA 2-3 eTCR), anti-BCMA4-anti-BCMA5 epsilon-TCR (Tandem BCMA 4-5 eTCR), anti-BCMA2-anti-BCMA3-anti-BCMA4 epsilon-TCR (Tandem BCMA 2-3-4 eTCR), anti-BCMA1-anti-BCMA4-anti-BCMA5 epsilon-TCR (Tandem BCMA 1-4-5 eTCR), as well as control untransduced cells was determined in a 20 h co-culture assay, where CHO/BCMA/CD19 cells (BCMA+CD19+) were co-cultured at effector-to-target cell ratios (E:T) of 0.5:1 and 1.5:1. Each condition was performed in triplicate, and the cytotoxicity of effector cells was detected by LDH assay kit (Roche). After 20 hr co-culture, the assay plate was centrifuged, and supernatant was collected and transferred to a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

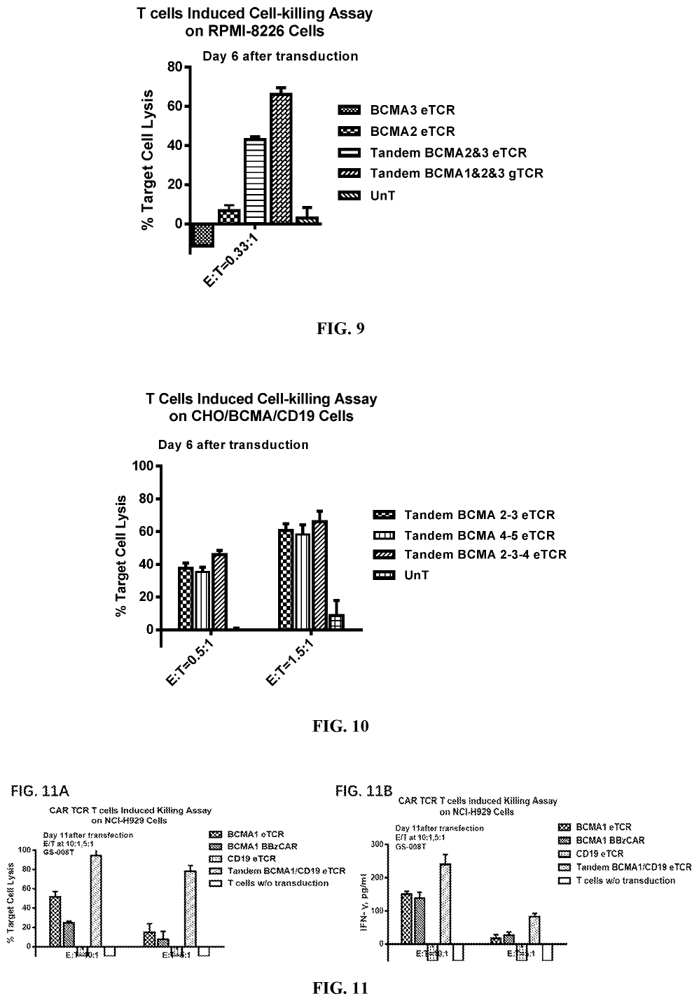

Results showed that constructs with five selected BCMA antibodies linked to eTCR in tandem combination all have superior in vitro cell-killing effects (as shown in FIG. 10).

Cytotoxicity of anti-BCMA1 epsilon-TCR (BCMA1 eTCR), anti-BCMA1 4-1BB-CD3zeta-CAR (BCMA1 BBzCAR), anti-CD19 epsilon-TCR (CD19 eTCR), and anti-BCMA1-anti-CD19-epsilon TCR (tandem BCMA1/CD19 eTCR), as well as untransduced control immune cells was determined in a 20 h co-culture assay. In the experiments, the effector cells were centrifugally collected, then diluted to the desired concentrations with 1640 phenol red free medium (Invitrogen) supplemented with 2% heat inactivated FBS (Invitrogen). The target cells, NCI-H929, exhibited strong expression of target antigen BCMA. The effector cells were co-cultured at different effector to target ratios (E:T=5:1 and 10:1 in) at 37° C. for 20 h in 96 well plate. Other wells contained assay buffer only (1640 phenol red free medium plus 2% hiFBS), target cell only (T), effector cell only (E) and max release of target cell (target cells with 1% solution of triton-X 100). Each condition was performed in triplicate, and the cytotoxicity of effector cells was detected by LDH assay kit (Roche). After 20 hr co-culture, the assay plate was centrifuged, and supernatant was collected and transferred to a new 96-w plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that effector cells expressing BCMA binding domain (e.g., anti-BCMA1), such as BCMA1 eTCR, BCMA1 BBzCAR, and tandem BCMA1/CD19 eTCR had greater cell killing activity as compared to the untransduced cell control and CD19 eTCR. Tandem BCMA1/CD19 eTCR showed greater cell killing activity as compared to BCMA1 eTCR or BCMA1 BBzCAR (as shown in FIG. 11A, day 11 after transfection).

IFN-7 expression was assayed by HTRF, as shown in FIG. 11B. 384-well low volume white plates were used in the assay for the IFN-γ detection (human IFNγ kit, Gisbio).

In a second multiple component cytotoxicity assay, anti-BCMA1-epsilon-TCR (BCMA1 eTCR), anti-BCMA1-4-1BB-CD3zeta-CAR (BCMA1 BBzCAR), anti-CD19-4-1BB-CD3zeta CAR (CD19 BBzCAR), anti-CD19-epsilon TCR (CD19 eTCR), anti-CD19-epsilon TCR/anti-BCMA1-4-1BB-CD3zeta CAR (CD19 eTCR/BCMA1 BBzCAR), and anti-BCMA1-epsilon TCR/anti-CD19-4-1BB-CD3 zeta CAR (BCMA1 eTCR/CD19 BBzCAR), as well as control untransduced cells were co-cultured with CHO-BCMA-CD19 cells (BCMA+ and CD19+) at effector-to-target cell ratios of 5:1, 10:1, and 20:1. Each condition was performed in triplicate, and the cytotoxicity of effector cells was detected by LDH assay kit (Roche). After 20 hr co-culture, the assay plate was centrifuged, and the supernatant was collected and transferred to a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that anti-BCMA and/or anti-CD-19 systems: anti-BCMA1 epsilon-TCR (BCMA eTCR), anti-BCMA1-4-1BB-CD3zeta-CAR (BCMA BBzCAR), anti-CD19-4-1BB-CD3zeta CAR (CD19 BBzCAR), anti-CD19-epsilon TCR (CD19 eTCR), anti-CD19-epsilon TCR-anti-BCMA1-4-1BB-CD3zeta CAR (CD19 eTCR/BCMA BBzCAR), anti-BCMA1-epsilon TCR/anti-CD19-4-1BB-CD3 zeta CAR (BCMA eTCR/CD19 BBzCAR) had greater cell killing activity as compared to untransduced control cells at an E:T ratio of 20:1. While in lower E:T ratio, especially 5:1, anti-CD19-epsilon TCR/anti-BCMA1-4-1BB-CD3zeta CAR (CD19 eTCR/BCMA BBzCAR), anti-BCMA1-epsilon TCR/anti-CD19-4-1BB-CD3 zeta CAR (BCMA eTCR/CD19 BBzCAR) showed greater cell-killing activity compared to single antibody fused CAR or TCR (as shown in FIG. 12A).

IFN-γ expression was assayed by HTRF, FIG. 12B. 384-well low volume white plates were used in the assay for the IFN-γ detection (human IFNγ kit, Gisbio).

In a third multiple component cytotoxicity assay, anti-CD19 epsilon-TCR(CD19 eTCR), anti-BCMA1-anti-CD19-epsilon TCR (tandem BCMA1/CD19 eTCR), anti-CD19-epsilon TCR/anti-BCMA1-delta TCR (CD19 eTCR/BCMA1 dTCR), anti-CD19-epsilon TCR/anti-BCMA1-4-1BB-CD3zeta CAR (CD19 eTCR/BCMA1 BBzCAR), and anti-BCMA1-epsilon TCR/anti-CD19-4-1BB-CD3zeta CAR (BCMA1 eTCR/CD19 BBzCAR), as well as control untransduced cells were co-cultured with CHO-BCMA-CD19 cells (BCMA+ and CD19+) at effector-to-target ratios of 10:1 and 5:1. Each condition was performed in triplicate, and the cytotoxicity of effector cells was detected by LDH assay kit (Roche). After 20 hr co-culture, the assay plate was centrifuged, and supernatant was collected and transferred to a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that anti-BCMA and anti-CD19 systems: anti-CD19 epsilon-TCR (CD19 eTCR), anti-BCMA1-anti-CD19-epsilon TCR (tandem BCMA1/CD19 eTCR), anti-CD19-epsilon TCR/anti-BCMA1-delta TCR (CD19 eTCR/BCMA1 dTCR), anti-CD19-epsilon TCR/anti-BCMA1-4-1BB-CD3zeta CAR (CD19 eTCR/BCMA1 BBzCAR), anti-BCMA1-epsilon TCR/anti-CD19-4-1BB-CD3zeta CAR (BCMA1 eTCR/CD19 BBzCAR) had anti-tumor activity as compared to the untransduced control cells. At higher E:T ratio (10:1), anti-BCMA1-anti-CD19-epsilon TCR (tandem BCMA1/CD19 eTCR), anti-BCMA1-epsilon TCR/anti-CD19-4-1BB-CD3zeta CAR (BCMA1 eTCR/CD19 BBzCAR) showed similar cell-killing activity, greater than anti-CD19 epsilon-TCR (CD19 eTCR) and anti-CD19-epsilon TCR/anti-BCMA1-delta TCR (CD19 eTCR/BCMA1 dTCR). At lower E:T ratio (5:1), anti-CD19 epsilon-TCR (CD19 eTCR) and anti-CD19-epsilon TCR/anti-BCMA1-delta TCR (CD19 eTCR/BCMA1 dTCR) showed almost no cell-killing activity, while anti-BCMA1-anti-CD19-epsilon TCR (tandem BCMA1/CD19 eTCR), anti-BCMA1-epsilon TCR/anti-CD19-4-1BB-CD3zeta CAR (BCMA1 eTCR/CD19 BBzCAR) still showed cell-killing effect with lysis of about 40% target cell (as shown in FIG. 13A).

In a fourth multiple component cytotoxicity assay, anti-BCMA and/or anti-CD19 systems: anti-BCMA1-epsilon TCR (BCMA1 eTCR), anti-BCMA1-4-1BB-CD3zeta CAR (BCMA1 BBzCAR), anti-BCMA1-anti-CD19-epsilon TCR (tandem BCMA1/CD19 eTCR), anti-CD19-epsilon TCR/anti-BCMA1-4-1BB-CD3zeta CAR (CD19 eTCR/BCMA1 BBzCAR), anti-BCMA1-epsilon TCR/anti-CD19-4-1BB-CD3zeta CAR (BCMA1 eTCR/CD19 BBzCAR) were co-cultured with NCI-H929 cells (BCMA+) at effector-to-target ratios of 2.5:1 and 5:1. Each condition was performed in triplicate, and the cytotoxicity of effector cells was detected by LDH assay kit (Roche). After 20 hr co-culture, the assay plate was centrifuged, and the supernatant was collected and transferred to a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that anti-BCMA and anti-CD19 systems: anti-BCMA1-epsilon TCR (BCMA1 eTCR), anti-BCMA1-4-1BB-CD3zeta CAR (BCMA1 BBzCAR), anti-BCMA1-anti-CD19-epsilon TCR (tandem BCMA1/CD19 eTCR), anti-CD19-epsilon TCR/anti-BCMA1-4-1BB-CD3zeta CAR (CD19 eTCR/BCMA1 BBzCAR), anti-BCMA1-epsilon TCR/anti-CD19-4-1BB-CD3 zeta CAR (BCMA1 eTCR/CD19 BBzCAR) had greater cell killing activity as compared to the untransduced controls (as shown in FIG. 13B).

In a fifth multiple component cytotoxicity assay, anti-BCMA and anti-CD19 systems: anti-BCMA1 epsilon-TCR (BCMA1 eTCR), anti-CD19-epsilon TCR/anti-BCMA1-gamma TCR (CD19 eTCR/BCMA1 gTCR), anti-CD19-epsilon TCR/anti-BCMA1-delta TCR (CD19 eTCR/BCMA1 dTCR), anti-CD19-epsilon TCR/anti-BCMA1-4-1BB-CD3zeta CAR (CD19 eTCR/BCMA1 BBzCAR), as well as control untransduced cells were co-cultured with CHO-BCMA-CD19 cells (BCMA+CD19+) at effector-to-target ratios of 1.3:1. Each condition was performed in triplicate, and the cytotoxicity of effector cells was detected by LDH assay kit (Roche). After 20 hr co-culture, the assay plate was centrifuged, and the supernatant was collected and transferred to a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that anti-BCMA1 epsilon-TCR (BCMA1 eTCR), anti-CD19-epsilon TCR/anti-BCMA1-gamma TCR (CD19 eTCR/BCMA1 gTCR), anti-CD19-epsilon TCR/anti-BCMA1-delta TCR (CD19 eTCR/BCMA1 dTCR), anti-CD19-epsilon TCR/anti-BCMA1-4-1BB-CD3zeta CAR (CD19 eTCR/BCMA1 BBzCAR) had greater cell killing activity as compared to the untransduced controls. Anti-CD19-epsilon TCR/anti-BCMA1-gamma TCR (CD19 eTCR/BCMA1 gTCR), anti-CD19-epsilon TCR/anti-BCMA1-delta TCR (CD19 eTCR/BCMA1 dTCR), anti-CD19-epsilon TCR/anti-BCMA1-4-1BB-CD3zeta CAR (CD19 eTCR/BCMA1 BBzCAR) had greater cell killing activity as compared to the anti-BCMA1 epsilon-TCR (BCMA1 eTCR). Anti-CD19-epsilon TCR/anti-BCMA1-4-1BB-CD3zeta CAR (CD19 eTCR/BCMA1 BBzCAR) had greater cell killing activity as compared to anti-CD19-epsilon TCR/anti-BCMA1-gamma TCR (CD19 eTCR/BCMA1 gTCR), anti-CD19-epsilon TCR/anti-BCMA1-delta TCR (CD19 eTCR/BCMA1 dTCR) (as shown in FIG. 14A). The results of FIG. 14B and FIG. 14C showed that the amount of IFNγ and TNFα secreted from T cells in the co-culture system had similar trends as the cell-killing effects.

In a sixth multiple component cytotoxicity assay, anti-BCMA and anti-CD19 systems: anti-BCMA1 epsilon-TCR (BCMA1 eTCR), anti-CD19-epsilon TCR/anti-BCMA1-4-1BB-CD3zeta CAR (CD19 eTCR/BCMA1 BBzCAR), anti-BCMA1 and anti-CD19-epsilon-TCR (Tandem BCMA1/CD19 dTCR), as well as control untransduced cells were co-cultured with CHO-BCMA-CD19 cells (BCMA+ and CD19+) at effector-to-target ratios of 1.3:1. Each condition was performed in triplicate, and the cytotoxicity of effector cells was detected by LDH assay kit (Roche). After 20 hr co-culture, the assay plate was centrifuged, and the supernatant was collected and transferred to a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that anti-BCMA1 epsilon-TCR (BCMA1 eTCR), anti-CD19-epsilon TCR/anti-BCMA1-4-1BB-CD3zeta CAR (CD19 eTCR/BCMA1 BBzCAR), and anti-BCMA1-anti-CD19-epsilon-TCR (Tandem BCMA1/CD19 dTCR) had greater cell killing activity as compared to the untransduced controls. Anti-CD19-epsilon TCR/anti-BCMA1-4-1BB-CD3zeta CAR (CD19 eTCR/BCMA1 BBzCAR), and anti-BCMA1-anti-CD19-epsilon-TCR (Tandem BCMA1/CD19 dTCR) had greater cell killing activity as compared to the anti-BCMA1 epsilon-TCR (BCMA1 eTCR) (as shown in FIG. 15A). The results of FIG. 15B and FIG. 15C showed that the amount of IFNγ and TNFα secreted from T cells in the co-culture system had similar trends as the cell-killing effects.

In a seventh multiple component cytotoxicity assay, anti-BCMA systems: anti-BCMA2 epsilon-TCR (BCMA eTCR), anti-BCMA2-epsilon TCR/anti-BCMA3-4-1BB-CD3zeta CAR (BCMA2 eTCR/BCMA3 BBzCAR), anti-BCMA2-gamma TCR/anti-BCMA3-4-1BB-CD3zeta CAR (BCMA2 gTCR/BCMA3 BBzCAR), anti-BCMA2-delta TCR/anti-BCMA3-4-1BB-CD3zeta CAR (BCMA2 dTCR/BCMA3 BBzCAR), as well as control untransduced cells were co-cultured with RPMI-8226 cells (BCMA+) at effector-to-target ratios of 0.5:1. Each condition was performed in triplicate, and the cytotoxicity of effector cells was detected by LDH assay kit (Roche). After 20 hr co-culture, the assay plate was centrifuged, and supernatant collected in a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

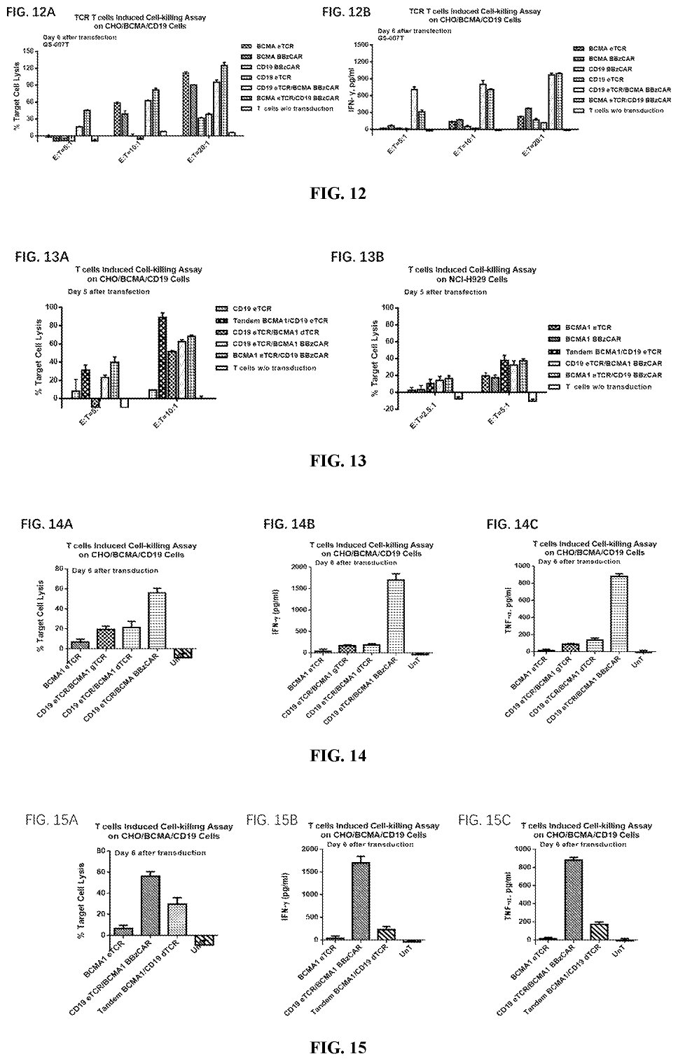

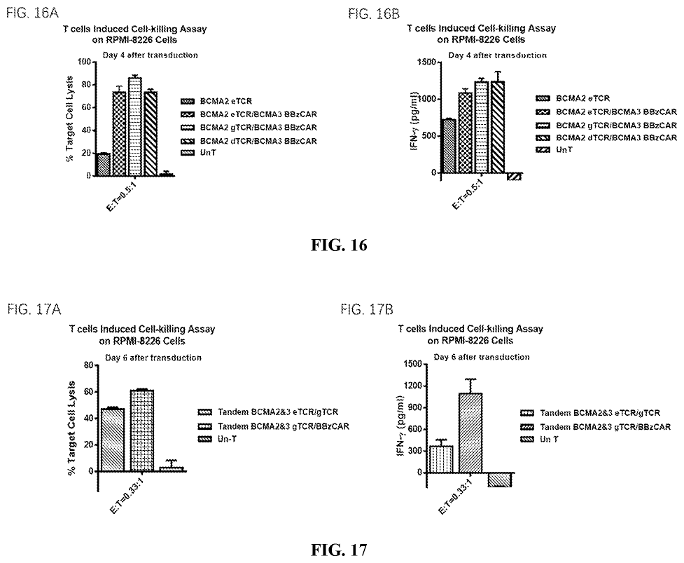

Results showed that anti-BCMA2 epsilon-TCR (BCMA2 eTCR), anti-BCMA2-epsilon TCR/anti-BCMA3-4-1BB-CD3zeta CAR (BCMA2 eTCR/BCMA3 BBzCAR), anti-BCMA2-gamma TCR/anti-BCMA3-4-1BB-CD3zeta CAR (BCMA2 gTCR/BCMA3 BBzCAR), anti-BCMA2-delta TCR/anti-BCMA3-4-1BB-CD3zeta CAR (BCMA2 dTCR/BCMA3 BBzCAR) had greater cell killing activity as compared to the untransduced controls. Anti-BCMA2-epsilon TCR/anti-BCMA3-4-1BB-CD3zeta CAR (BCMA2 eTCR/BCMA3 BBzCAR), anti-BCMA2-gamma TCR/anti-BCMA3-4-1BB-CD3zeta CAR (BCMA2 gTCR/BCMA3 BBzCAR), anti-BCMA2-delta TCR/anti-BCMA3-4-1BB-CD3zeta CAR (BCMA2 dTCR/BCMA3 BBzCAR) had significantly greater cell killing activity as compared to the anti-BCMA2 epsilon-TCR (BCMA2 eTCR), as shown in FIG. 16A. FIG. 16B shows the amount of IFNγ secreted from T cells in the co-culture system.

In an eighth multiple component cytotoxicity assay, anti-BCMA systems: anti-BCMA2-anti-BCMA3 epsilon-TCR/anti-BCMA2-anti-BCMA3 gamma-TCR (tandem BCMA2&3 eTCR/gTCR), anti-BCMA2-anti-BCMA3 gamma-TCR/anti-BCMA2-anti-BCMA3 4-1BB-CD3zeta CAR (tandem BCMA2&3 gTCR/BBzCAR), as well as control untransduced cells were co-cultured with RPMI-8226 cells (BCMA+) at effector-to-target ratios of 0.33:1. Each condition was performed in triplicate, and the cytotoxicity of effector cells was detected by LDH assay kit (Roche). After 20 hr co-culture, the assay plate was centrifuged, and supernatant was collected and transferred to a new 96-well plate. The supernatant plate was diluted with an equal volume of the LDH assay reagent according to the manufacture's manual. The assay plate was incubated for about 30 min at 15° C.˜25° C. The absorbance of the plate was measured at 492 nm and 650 nm using Flexstation reader (Molecular Devices) and calculated.

Results showed that anti-BCMA2-anti-BCMA3 epsilon-TCR/anti-BCMA2-anti-BCMA3 gamma-TCR (tandem BCMA2&3 eTCR/gTCR), anti-BCMA2-anti-BCMA3 gamma-TCR/anti-BCMA2-anti-BCMA3 4-1BB-CD3zeta CAR (tandem BCMA2&3 gTCR/BBzCAR) had greater cell killing activity as compared to the untransduced controls. Anti-BCMA2-anti-BCMA3 gamma-TCR/anti-BCMA2-anti-BCMA3 4-1BB-CD3zeta CAR (tandem BCMA2&3 gTCR/BBzCAR) had significantly greater cell killing activity as compared to the anti-BCMA2-anti-BCMA3 epsilon-TCR/anti-BCMA2-anti-BCMA3 gamma-TCR (tandem BCMA2&3 eTCR/gTCR), as shown in FIG. 17A. FIG. 17B shows the amount of IFNγ secreted from T cells in the co-culture system.

### Example 7: Cell Cytotoxicity Assay (Luciferase Assay)

To evaluate the cytotoxicity of modified immune cells expressing any of the systems provided herein, CAR-T cells, TCR-T cells, and un-transfected T cells (UnT) were centrifugally collected and diluted to desired concentrations utilizing 1640 phenol red free medium (Invitrogen) supplemented with 2% heat inactivated FBS (Invitrogen). Tumor cells exhibiting strong expression of BCMA and luciferase were used as target cells. The TCR-T cells or CAR-T cells and target cells were co-cultured at different effector to target ratios (E:T) at 37° C. for 20 h in a 96 well plate. Other wells contained controls conditions: target cell only (T) and max release of target cell (1% solution of triton-X 100). Each condition was performed in triplicate, and the cytotoxicity of CAR-T cells was detected utilizing the One-Glo assay kit (Promega).

After 20 hour co-culture, the assay plate was centrifuged and an equal volume of the One-Glo assay reagent was added according to the manufacturer's instructions. The plate was incubated for about 3 min at room temperature. Post incubation, the luciferase signal was measured using a PheraStarplus reader (BMG labtech). The percentage of tumor cell lysis was calculated using the formula:

% Target cell lysis=(1−(RLUE:T-RLUMax release)/(RLUT−RLUMax release))*100.

### Example 8: Cytokine Release Detection (IFNγ& TNFα)

The supernatant of the cytotoxicity assay plate was collected for cytokine release analysis (Human IFN gamma kit, Cisbio, Cat #62HIFNGPEH, Human TNF alpha kit, Cisbio, Cat #62HTNFAPEH), Human IL6 kit, Cisbio, Cat #62HIL06PEG, and Human IL2 kit, Cisbio, Cat #62HIL02PEH). The cell supernatant and a standard were dispensed directly into the assay plate for the cytokine detection utilizing HTRF® reagents. The antibodies labeled with the HTRF donor and acceptor were pre-mixed and added in a single dispensing step. The HTRF standard curve was generated using the 4 Parameter Logistic (4PL) curve. The standard curve regression enabled the accurate measurement of an unknown sample concentration across a wider range of concentrations than linear analysis, making it suitable for analysis of biological systems such as cytokine release. Applicable assay kits include Human IFN gamma kit, Cisbio, Cat #62HIFNGPEH; Human TNF alpha kit, Cisbio, Cat #62HTNFAPEH; Human IL6 kit, Cisbio, Cat #62HIL06PEG; and Human IL2 kit, Cisbio, Cat #62HIL02PEH.

### Example 9: In Vivo Efficacy

### In Vivo Efficacy of BCMA CAR-TCR-T by a Multiple Myeloma Tumor Xenograft

In vivo anti-tumor efficacy of CAR-TCR-T cells was evaluated in a NCG mouse model (NOD_Prkdcem26Cd52/NjuCrl) having a multiple myeloma tumor xenograft.

The NCG mouse model was generated by sequential CRISPR/Cas9 editing of the Prkdc and I12rg loci in the NOD/Nju mouse, providing a mouse coisogenic to the NOD/Nju. The NOD/Nju mouse carries a mutation in the Sirpa (SIRPα) gene that allows for engrafting of foreign hematopoietic stem cells. The Prkdc knockout generates a SCID-like phenotype lacking proper T-cell and B-cell formation. The knockout of the I12rg gene further exacerbates the SCID like phenotype while additionally resulting in a decrease of NK cell production. Thus, the NCG mouse is a “triple-immunodeficient” mouse strain that is more immunocompromised than commonly used immunodeficient mouse strains including SCID and nude mice. Prkdc and I12rg are part of the SCID (severe combined immunodeficiency) family of genes affecting maturation and formation of T cells, B cells, NK cells and, to a lesser degree, dendritic cells. Prkdc encodes the catalytic subunit of the DNA-dependent protein kinase enzyme, which is required for V(D)J recombination, a necessary process to propagate antibody diversity in maturing T and B cells. I12rg encodes the common gamma subunit found in IL-2 and multiple IL receptors (IL-4, IL-7, IL-9, IL-15 and IL-21), which are required to induce cytokine-mediated signaling for maturation of immature lymphocytes (e.g. T, B and NK cells) and other leukocytes. BCMA CAR-T cells were prepared using T cells from various donors to screen for T cell source yielding CAR-T with the highest efficacy of killing RPMI8226-Luc cells in vitro. CAR-T cells were prepared using T cells of the selected donor for in vivo animal assays. To create the tumor xenograft, NCG mice were injected intravenously with RPMI8226-Luc cells. Fourteen days later, tumor engrafted mice were treated with the BCMA CAR-T cells (1.5e5 positive cells) or un-transduced T cells, followed by in vivo bioluminescence imaging (BLI).

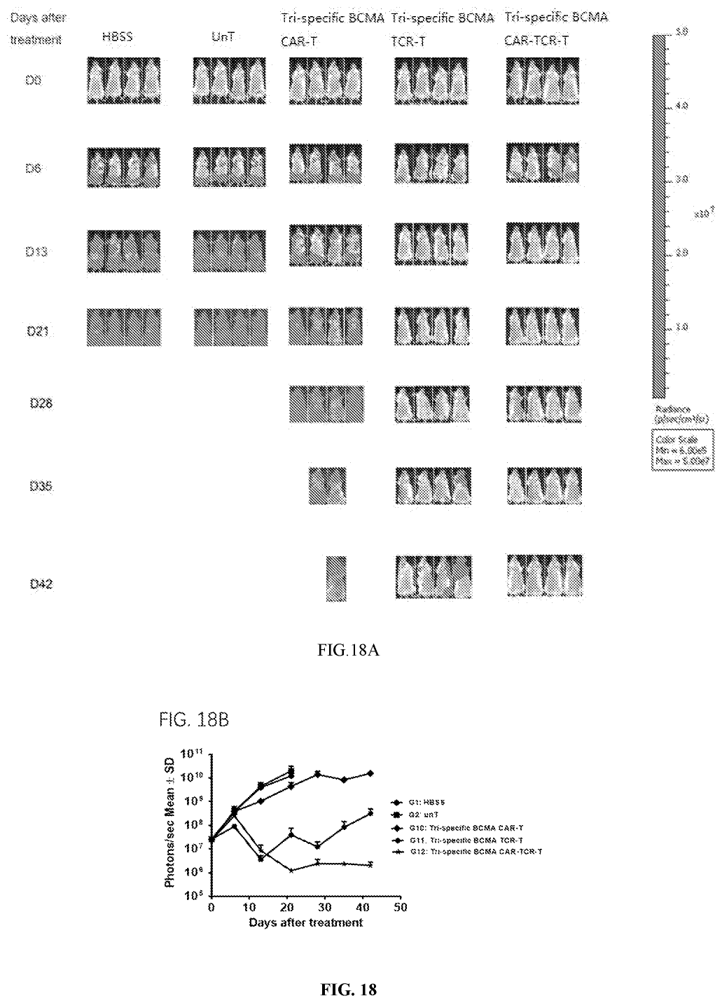

Anti-BCMA1-anti-BCMA2-anti-BCMA3 BBzCAR (tri-specific BCMA CAR-T) cells, anti-BCMA1-anti-BCMA2-anti-BCMA3 eTCR (tri-specific BCMA TCR-T) cells and anti-BCMA2 eTCR/anti-BCMA1-anti-BCMA3 BBzCAR (tri-specific BCMA CAR-TCR-T) cells were evaluated in a NCG mouse model (NOD_Prkdc^{em26Cd52}/NjuCrl) having a multiple myeloma tumor xenograft. Anti-BCMA2 eTCR/anti-BCMA1-anti-BCMA3 BBzCAR (tri-specific BCMA CAR-TCR-T) showed great anti-tumor activity in low dose, as shown in FIG. 18.

### In Vivo Efficacy of MSLN FSHR CAR-TCR-T by OVCAR-8 Xenograft Model in NSG Mice

Anti-tumor activity of anti-mesothelin CAR-T was assessed in vivo in an OVCAR-8 xenograft model. 10×10⁶ OVCAR-8 cells were implanted subcutaneously on day 0 in NOD scid gamma (NSG) mice. Once tumors were 150-200 mm³, the mice were randomized into treatment groups. 1e5 CAR positive T cells in a 200 μl dose was administered intravenously. Mice and tumors were monitored for about 60 days after tumor cell implantation.

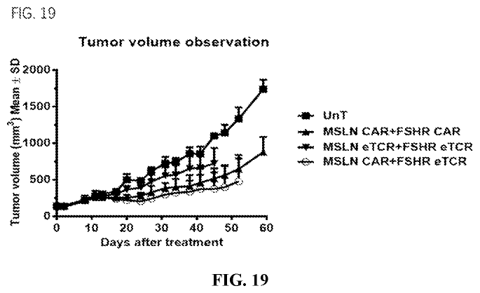

Results showed that anti-MSLN/FSHR double CAR-T (MSLN CAR+FSHR CAR), anti-MSLN/FSHR double eTCR-T (MSLN eTCR+FSHR eTCR) and anti-FSHR eTCR/MSLN CAR-T (FSHR eTCR+MSLN CAR) had different anti-tumor activities in vivo, and anti-FSHR eTCR/MSLN CAR-T (FSHR eTCR+MSLN CAR) showed greater anti-tumor activity compared to anti-MSLN/FSHR double CAR-T (MSLN CAR+FSHR CAR), anti-MSLN/FSHR double eTCR-T (MSLN eTCR+FSHR eTCR) in low dose (as shown in FIG. 19).

### Example 10: Rapid Expansion Protocol

In order to generate a large number of transduced cells, T cells were induced to proliferate by using a rapid expansion protocol (REP). Prior to use in REPs, T cells were cultured with anti-CD3, anti-CD28 and IL-2 at the beginning and transduced on the second day. The cells were cultured in a 75 cm² flask at 37° C. and 5% C02. The cells were counted and suspended at a concentration of 0.5×10⁶ cells/mL in fresh T cell medium supplemented with 300 IU/mL of IL-2 every two days, and for the remainder of the time, they would be kept in culture.

A wide variety of antigen binding domain sequences are applicable for constructing the vectors constructs and systems disclosed herein, see e.g., WO2017/025038, which is incorporated herein in its entirety (BCMA2 to BCMA4, BCMA 14 to BCMA 21).

Non-limiting exemplary sequences are shown in the Tables 10 and 11 as follows:

TABLE 10
Exemplary Sequences

| SEQ ID | | |
|---|---|---|
| NO | Ab code | Sequence(the CDRs of new anti-BCMA sdAbs are underlined) |
| 1 | human BCMA | MLQMAGQCSQNEYFDSLLHACIPCQLRCSSNTPPLTCQRYCNA |
| | extracellular | SVTNSVKGTNA |
| | domain (ECD) | |
| 2 | cynomolgus | MLQMARQCSQNEYFDSLLHDCKPCQLRCSSTPPLTCQRYCNAS |
| | BCMA ECD | MTNSVKGMNA |
| 3 | BCMA2 | EVQLVESGGGLVQAGGSLRLSCAASGRTFTMGWFRQAPGKER |
| | | EFVAAISLSPTLAYYAESVKGRFTISRDNAKNTVVLQMNSLKPE |
| | | DTALYYCAADRKSVMSIRPDYWGQGTQVTVSS |
| 4 | BCMA3 | QVKLEESGGGLVQAGRSLRLSCAASEHTFSSHVMGWFRQAPG |
| | | KERESVAVIGWRDISTSYADSVKGRFTISRDNAKKTLYLQMNSL |
| | | KPEDTAVYYCAARRIDAADFDSWGQGTQVTVSS |
| 5 | BCMA4 | AVQLVESGGGLVQAGDSLRLTCTASGRAFSTYFMAWFRQAPG |
| | | KEREFVAGIAWSGGSTAYADSVKGRFTISRDNAKNTVYLQMNS |
| | | LKSEDTAVYYCASRGIEVEEFGAWGQGTQVTVSS |
| 6 | BCMA14 | QVQLVESGGGLVQPGGSLRLSCEASGFTLDYYAIGWFRQAPGK |
| | | EREGVICISRSDGSTYYADSVKGRFTISRDNAKKTVYLQMISLKP |
| | | EDTAAYYCAAGADCSGYLRDYEFRGQGTQVTVSS |
| 7 | BCMA15 | QVKLEESGGRLVQPRGSLRLSCAGSGRTFSTYGMAWFRQAPGK |
| | | EREFVASKASMNYSGRTYYADSVKGRFTIARDNAKNMVFLQM |
| | | NNLKPEDTAVYYCAAGTGCSTYGCFDAQIIDYWGKGTLVTVSS |
| 8 | BCMA16 | AVQLVDSGGGLVQPGGSLRLSCVASGGIFVINAMGWYRQAPG |
| | | KQRELVASIRGLGRTNYDDSVKGRFTISRDNANNTVYLQMNSL |
| | | EPEDTAVYYCTVYVTLLGGVNRDYWGQGTQVTVSS |
| 9 | BCMA17 | EVQLVESGGGLVQAGGSLRLSCAASGRTFSSIVMGWFRQAPGK |
| | | EREFVGAIMWNDGITYLQDSVKGRFTIFRDNAKNTVYLQMNSL |
| | | KLEDTAVYYCAASKGRYSEYEYWGQGTQVTVSS |
| 10 | BCMA18 | EVQLVESGGGVVQAGGSLTVSCTASGFTFDRAVIVWFRQAPGK |
| | | GREGVSFIKPSDGTIYYIDSLKGRFTISSDIAKNTVYLQMKSLESE |
| | | DSAVYYCAASPEDWYTDWIDWSIYRWQHWGQGTQVTVSS |
| 11 | BCMA19 | EVQLVESGGGMVQAGDSLRLSCVQSTYTVNSDVMGWFRQAP |
| | | GKEREFVGAIMWNDGITYLQDSVKGRFTIFRDNAKNTVYLQM |
| | | NSLKLEDTAVYYCAASKGRYSEYEYWGQGTQVTVSS |
| 12 | BCMA20 | AVQLVESGGGLVQAGDSLRLSCTASGATLTNDHMAWFRQAPG |
| | | KGREFVAAIDWSGRTTNYADPVEGRFTISRNNAKNTVYLEMNS |
| | | LKLEDTAVYYCAVLRAWISYDNDYWGQGTQVTVSS |
| 13 | BCMA21 | QVQLVESGGGLVQAGGSLRLSCAASGGTLSKNTVAWFRQAPG |
| | | KERGFVASITWDGRTTYYADSVKGRFTISRDNAKNTVYLQMNS |
| | | LKPEDTAVYVCADLGKWPAGPADYWGQGTQVTVSS |
| 14 | BCMA1 | QVQLVESGGGSVQAGGSLRLSCKASGAIYDTNCMAWFRQTPG |
| | | KEREGVATIDLGNPITYYADSVKGRFTISRDNAKNTMYLQMNS |
| | | LEPEDTAMYYCAATSWWPCTTFNAGYANWGQGTQVTVSS |
| 15 | BCMA5 | QVQLEESGGGSVQAGGSLRLSCAYTYSTYSNYYMGWFREAPG |
| | | KARTSVAIISSDTTITYKDAVKGRFTISKDNAKNTLYLQMNSLK |
| | | PEDSAMYRCAAWTSDWSVAYWGQGTQVTVSS |
| 16 | BCMA6 | QMQLVESGGGSVQAGGSLRLSCTASGYTFDDSAMGWYRQAPG |
| | | NECELVSSISSDGSTYYSDSVKGRFTISQDNAKNTMYLQMNSLK |
| | | PEDTAVYSCAASSGEDGGSWSTPCHFFGYWGQGTQVTVSS |
| 17 | BCMA7 | QVHLMESGGGSVQSGGSLRLSCAASGYTYSSYCMAWFRQAPG |
| | | KEREGVAAIASDGSTYYTDSVKGRFTISQDNAKNTLYLQMNSL |
| | | KPEDTAMYYCGADPVGCSWPDYWGQGTQVTVSS |
| 18 | BCMA8 | QVQLVESGGGSVQAGGSLRLSCAASGGTRSWNYMAWFRQAP |
| | | GKEREDVAIIDNVGSTRYADSVKGRFTISQDTAQNTLYLQMNS |
| | | LKPEDTAMYYCAARVSWCEDPPCGFDYWGQGTQVTVSS |
| 19 | BCMA9 | QVQLVESGGGSVQAGGSLRLSCKSSGAPYSSNCMAWFRQTPG |
| | | KGREGVATIDLASHDTYYADSVKGRFTISRDNAKNTMYLQMN |
| | | SLKPEDTAMYYCAATSWWPCTTFNGGYANWGQGTQVTVSS |
| 20 | BCMA10 | QVQLAESGGGLVQPGGSLRLSCAGSGFTFSSYDMNWVRQAPG |
| | | KGLERVSTTFNGDDGTNYADSVLGRFTASRDKAKNTLYLQMN |
| | | SLKTEDTAVYYCAAAVPGVDWYDTTRYKYWGQGTQVTVSS |
| 21 | BCMA11 | QVQLVESGGGVVQPGGSLRLSCAASGFAFSNYAMTWGRQAPG |
| | | QRLEWVSTIDSGGGSTTYSDSVKGRFTISRDNAKNTLYLQLNNL |
| | | KSEDTAVYYCSENVDCNGDYCYRANYWGQGTQVTVSS |
| 22 | BCMA12 | QVHLVESGGGSVQAGGSLRLSCKSSGATYSSNCMAWFRQTPG |
| | | KEREGVATIDLASHGTYYADSVKGRFTISRDNAKNTMYLQMSG |
| | | LRPEDTALYYCAATSWWPCTTFNGGYASWGQGTQVTVSS |
| 23 | BCMA13 | QVHLVESGGGSVQAGGSLRLSCKASGAIYDTNCMAWFRQTPG |
| | | KEREGVATIDLGNPITYYADSVKGRFTISRDNAKNTMYLQMNS |
| | | LKPEDTAMYYCAATSWWPCPANNVGYANWGQGTQVTVSS |
| 24 | CD8α signal | MALPVTALLLPLALLLHAARP |
| | peptide amino | |
| | acid sequence | |
| 25 | CD8α hinge | TTTPAPRPPTPAPTIASQPLSLRPEACRPAAGGAVHTRGLDFACD |
| | amino acid | |
| | sequence | |
| SEQ ID | CD8α | IYIWAPLAGTCGVLLLSLVITLYC |
| NO. 26 | transmembrane | |
| | domain amino | |
| | acid sequence | |
| 27 | 4-1BB | KRGRKKLLYIFKQPFMRPVQTTQEEDGCSCRFPEEEEGGCEL |
| | intracellular | |
| | domain amino | |
| | acid sequence | |
| 28 | P2A element | GSGATNFSLLKQAGDVEENPGP |
| | amino acid | |
| | sequence | |
| 29 | CD3ζ | RVKFSRSADAPAYKQGQNQLYNELNLGRREEYDVLDKRRGRD |
| | intracellular | PEMGGKPRRKNPQEGLYNELQKDKMAEAYSEIGMKGERRRGK |
| | domain amino | GHDGLYQGLSTATKDTYDALHMQALPPR |
| | acid sequence | |
| 30 | CD3ϵ signal | MQSGTHWRVLGLCLLSVGVWGQ |
| | peptide amino | |
| | acid sequence | |
| 31 | CD3ϵ | DGNEEMGGITQTPYKVSISGTTVILTCPQYPGSEILWQHNDKNIG |
| | extracellular | GDEDDKNIGSDEDHLSLKEFSELEQSGYYVCYPRGSKPEDANF |
| | domain (ECD), | YLYLRARVCENCMEMDVMSVATIVIVDICITGGLLLLVYYWSK |
| | transmembrane | NRKAKAKPVTRGAGAGGRQRGQNKERPPPVPNPDYEPIRKGQ |
| | domain and | RDLYSGLNQRRI |
| | intracellular | |
| | domain amino | |
| | acid sequence | |
| 32 | CD3γ signal | MEQGKGLAVLILAIILLQGTLA |
| | peptide amino | |
| | acid sequence | |
| 33 | CD3γ | QSIKGNHLVKVYDYQEDGSVLLTCDAEAKNITWFKDGKMIGFL |
| | extracellular | TEDKKKWNLGSNAKDPRGMYQCKGSQNKSKPLQVYYRMCQN |
| | domain (ECD). | CIELNAATISGFLFAEIVSIFVLAVGVYFIAGQDGVRQSRASDKQ |
| | transmembrane | TLLPNDQLYQPLKDREDDQYSHLQGNQLRRN |
| | domain and | |
| | intracellular | |
| | domain amino | |
| | acid sequence | |
| 34 | CD3δ signal | MEHSTFLSGLVLATLLSQVSP |
| | peptide amino | |
| | acid sequence | |
| 35 | CD3δ | FKIPIEELEDRVFVNCNTSITWVEGTVGTLLSDITRLDLGKRILDP |
| | extracellular | RGIYRCNGTDIYKDKESTVQVHYRMCQSCVELDPATVAGIIVT |
| | domain (ECD), | DVIATLLLALGVFCFAGHETGRLSGAADTQALLRNDQVYQPLR |
| | transmembrane | DRDDAQYSHLGGNWARNK |
| | domain and | |
| | intracellular | |
| | domain amino | |
| | acid sequence | |
| 36 | Linker amino acid | GGGGS |
| | sequence (short) | |
| 37 | Linker amino acid | GGGGSGGGGSGGGGS |
| | sequence (long) | |

TABLE 11
Sequences of anti-CD19 V_{H}H

| SEQ ID NO | Sequence |
|---|---|
| 38 | QVKLEESGGELVQPGGPLRLSCAASGNIFSINRMGWYRQAPGKQRAFVAS |
| | ITVRGITNYADSVKGRFTISVDKSKNTIYLQMNALKPEDTAVYYCNAVSSN |
| | RDPDYWGQGTQVTVSS |
| 39 | QVKLEESGGGLVQAGESLRLSCAASGHTLSAYTMGWFRQAPEREREFVA |
| | AITRSGGRTSYGDSVKGRFTISRDTAKNTVYLQMNSLKPEDTAVYYCAAD |
| | LRYRTVVNGLADYWGQGTQVTVSS |
| 40 | QVKLEESGGGLVQAGGSLRLSCAASGRSFSNYDMGWFRQAPGKEREFVA |
| | RISRRGDSTYYADSVKGRFIISRDNAKNTVYLQMNSLKPEDTAVYYCAAR |
| | WRGSREIDYWGQGTQVTVSS |

TABLE 12
Sequences of anti-MSLN scFv and FSH β 33-53

| SEQ ID | | |
|---|---|---|
| NO | Ab code | Sequence |
| 41 | anti- | EVQLVESGGGLVQPGGSLRLSCAASGFNLYYYSIHWVRQAPGK |
| | MSLN | GLEWVAYISSSSSYTYYADSVKGRFTISADTSKNTAYLQMNSLR |
| | scFv | AEDTAVYYCARYYPYYGMDYWGQGTLVTVSSGGGGSGGGGS |
| | | GGGGSDIQMTQSPSSLSASVGDRVTITCRASQSVSSAVAWYQQ |
| | | KPGKAPKLLIYSASSLYSGVPSRFSGSRSGTDFTLTISSLQPEDFA |
| | | TYYCQQGFSYYPITFGQGTKVEIK |
| 42 | anti- | EVQLVESGGGLVQPGGSLRLSCAASGFNIYYSSMHWVRQAPGK |
| | MSLN | GLEWVAYIYPYYSYTYYADSVKGRFTISADTSKNTAYLQMNSL |
| | scFv | RAEDTAVYYCARGYALDYWGQGTLVTVSSGGGGSGGGGSGG |
| | | GGSDIQMTQSPSSLSASVGDRVTITCRASQSVSSAVAWYQQKP |
| | | GKAPKLLIYSASSLYSGVPSRFSGSRSGTDFTLTISSLQPEDFATY |
| | | YCQQASSGYHYLITFGQGTKVEIK |
| 43 | anti- | EVQLVESGGGLVQPGGSLRLSCAASGFNIYSSSIHWVRQAPGKG |
| | MSLN | LEWVASISSYSSYTSYADSVKGRFTISADTSKNTAYLQMNSLRA |
| | scFv | EDTAVYYCARYYAMDYWGQGTLVTVSSGGGGSGGGGSGGGG |
| | | SDIQMTQSPSSLSASVGDRVTITCRASQSVSSAVAWYQQKPGK |
| | | APKLLIYSASSLYSGVPSRFSGSRSGTDFTLTISSLQPEDFATYYC |
| | | QQGPYYHPITFGQGTKVEIK |
| 44 | anti- | EVQLVESGGGLVQPGGSLRLSCAASGFNLSYSSIHWVRQAPGK |
| | MSLN | GLEWVASIYSYSGSTYYADSVKGRFTISADTSKNTAYLQMNSL |
| | scFv | RAEDTAVYYCARYWGMDYWGQGTLVTVSSGGGGSGGGGSG |
| | | GGGSDIQMTQSPSSLSASVGDRVTITCRASQSVSSAVAWYQQK |
| | | PGKAPKLLIYSASSLYSGVPSRFSGSRSGTDFTLTISSLQPEDFAT |
| | | YYCQQYYWYYPITFGQGTKVEIK |
| 45 | anti- | EVQLVESGGGLVQPGGSLRLSCAASGFNLYSYYMHWVRQAPG |
| | MSLN | KGLEWVASIYSYSSYTSYADSVKGRFTISADTSKNTAYLQMNS |
| | scFv | LRAEDTAVYYCARPFGWGYAGMDYWGQGTLVTVSSGGGGSG |
| | | GGGSGGGGSDIQMTQSPSSLSASVGDRVTITCRASQSVSSAVA |
| | | WYQQKPGKAPKLLIYSASSLYSGVPSRFSGSRSGTDFTLTISSLQ |
| | | PEDFATYYCQQGYAPITFGQGTKVEIK |
| 46 | FSHβ | YTRDLVYKDPARPKIQKTCTF |
| | 33-53 | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for inducing activity of an immune cell, comprising:
(a) a chimeric antigen receptor (CAR) comprising a first antigen binding domain which exhibits specific binding to a first epitope, a transmembrane domain, and an intracellular signaling domain; and
(b) a modified T cell receptor (TCR) complex comprising a second antigen binding domain which exhibits specific binding to a second epitope, wherein said second antigen binding domain is linked to:
(i) at least one TCR chain selected from an alpha chain, a beta chain, a gamma chain and a delta chain of a T cell receptor,
(ii) an epsilon chain, a delta chain, and/or a gamma chain of cluster of differentiation 3 (CD3), or
(iii) a CD3 zeta chain.

2. The system of claim 1, wherein binding of the first antigen binding domain to the first epitope, and binding of the second antigen binding domain to the second epitope activates an immune cell activity of an immune cell expressing the system.

3-124. (canceled)

125. The system of claim 1, wherein binding of the first antigen binding domain to the first epitope, or binding of the second antigen binding domain to the second epitope activates an immune cell activity of an immune cell expressing the system.

126. The system of claim 1, wherein said modified TCR comprises one or more additional antigen binding domains that are linked to (i) at least one TCR chain selected from an alpha chain, a beta chain, a gamma chain and a delta chain of a T cell receptor, (ii) an epsilon chain, a delta chain, or a gamma chain of cluster of differentiation 3 (CD3), or (iii) a CD3 zeta chain, wherein binding of said one or more additional antigen binding domains to their respective epitopes activates an immune cell activity of an immune cell expressing the system.

127. The system of claim 126, wherein said second antigen binding domain and said one or more additional antigen binding domains are linked in tandem.

128. The system of claim 1, wherein said CAR comprises one or more additional antigen binding domains, wherein said one or more additional antigen binding domains exhibit specific binding to one or more additional epitopes, and wherein said one or more additional epitopes are the same as the first or second epitope or different from the first and second epitope.

129. The system of claim 128, wherein said one or more additional antigen binding domains and the first antigen binding domain are linked in tandem.

130. The system of claim 1, wherein said intracellular signaling domain of said CAR comprises an immunoreceptor tyrosine-based activation motif (ITAM), an immunoreceptor tyrosine-based inhibition motif (ITIM), or an signaling domain of an Fcγ receptor (FcγR), an Fcε receptor (FcεR), an Fcα receptor (FcαR), neonatal Fc receptor (FcεRn), CD3, CD3ζ, CD3γ, CD3δ, CD3ε, CD4, CD5, CD8, CD21, CD22, CD28, CD32, CD40L (CD154), CD45, CD66d, CD79a, CD79b, CD80, CD86, CD278 (also known as ICOS), CD247ζ, CD247η, DAP10, DAP12, FYN, LAT, Lck, MAPK, MHC complex, NFAT, NF-κB, PLC-γ, iC3b, C3dg, C3d, or Zap70.

131. The system of claim 1, wherein said CAR further comprises a co-stimulatory domain, wherein the co-stimulatory domain comprises a signaling domain of a MHC class I molecule, a TNF receptor protein, an immunoglobulin-like protein, a cytokine receptor, an integrin, a signaling lymphocytic activation molecule (SLAM protein), an activating NK cell receptor, a Toll ligand receptor, or a molecule selected from the group consisting of 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF-R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8a, CD80, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2Rβ, IL2Rγ, IL7Rα, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6.

132. The system of claim 1, wherein said first antigen binding domain or said second antigen binding domain comprises:
i) a Fab, a Fab′, a F(ab′)₂, an Fv, a single-chain Fv (scFv), minibody, a diabody, a single-domain antibody, a light chain variable domain (VL), or a variable domain (V_{H}H) of camelid antibody; or
ii) a receptor or a ligand for a receptor.

133. The system of claim 1, wherein said first epitope and said second epitope are present on different antigens, or on a common antigen.

134. The system of claim 1, wherein said first epitope or said second epitope i) is present on a universal antigen, ii) is present on one or more cell surface antigens, iii) is present on a neoantigen, iv) is present on a tumor-associated antigen, an immune checkpoint receptor or immune checkpoint receptor ligand, v) is present on a cytokine or a cytokine receptor, or vi) is present on an antigen presented by a major histocompatibility complex (MHC).

135. The system of claim 134, wherein the tumor-associated antigen is selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD4, CD23, CD24, CD30, CD33, CD38, CD44v7/8, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFαRII, TGFβRII, TP1, TRAG-3, TRG, TRP-1, TRP-2, TRP-2/INT2, TRP-2-6b, Tyrosinase, VEGF-R2, WT1, α-folate receptor, and κ-light chain,
wherein the immune checkpoint receptor or immune checkpoint receptor ligand is PD-1, PD-L1, PD-L2, CTLA-4, TIM-3, LAG3, TIGIT, BLTA, CD47 or CD40,
wherein the cytokine or cytokine receptor is CCR2b, CXCR2 (CXCL1 receptor), CCR4 (CCL17 receptor), Gro-a, IL-2, IL-7, IL-15, IL-21, IL-12, Heparanase, CD137L, LEM, Bcl-2, CCL17, CCL19 or CCL2,
wherein the MHC is HLA class 1 or HLA class 2.

136. The system of claim 1, wherein said first antigen binding domain or the second antigen binding domain comprises
i) the amino acid sequence selected from the group consisting of SEQ ID NOs: 3-23, and 38 to 46, or
ii) the formula: A-X-B-Y-C-Z-D,
wherein:
X comprises an amino acid sequence having at least 80% or at least 90% identity to any one selected from the group consisting of SEQ ID NOs: 87-96;
Y comprises an amino acid sequence having at least 80% or at least 90% identity to any one selected from the group consisting of SEQ ID NOs: 97-106; and
Z comprises an amino acid sequence having at least 80% or at least 90% identity to any one selected from the group consisting of SEQ ID NOs: 107-116.

137. A method of inducing activity of an immune cell against a target cell, comprising:
(a) expressing a system according to claim 1 in an immune cell; and
(b) contacting a target cell with the immune cell under conditions that induce said activity of the immune cell against the target cell.

138. The method of claim 137, wherein said activity is cytotoxicity, and said cytotoxicity of the immune cell induces death of the target cell, wherein target cell is selected from a cancer cell, a hematopoietic cell, a solid tumor cell, and a cell identified in one or more of heart, blood vessels, salivary gland, esophagus, stomach, liver, gallbladder, pancreas, intestine, colon, rectum, anus, endocrine gland, adrenal gland, kidney, ureter, bladder, lymph node, tonsils, adenoid, thymus, spleen, skin, muscle, brain, spinal cord, nerve, ovary, fallopian tube, uterus, vagina, mammary gland, testes, prostate, penis, pharynx, larynx, trachea, bronchi, lung, diaphragm, cartilage, ligaments, and tendon.

139. A genetically modified immune cell expressing the system of claim 1.

140. The genetically modified immune cell according to claim 139, wherein the immune cell is a lymphocyte.

141. The genetically modified immune cell according to claim 140, wherein the lymphocyte is a natural killer (NK) cell, an effector T cell, a memory T cell, a cytotoxic T cell, a NKT cell, a T helper cell, a KHYG-1 cell, a α/β T cell, a γ/δ T cell, a CD4⁺ T cell or CD8⁺ T cell.

142. A method of treating a cancer in a subject, comprising administering to the subject an effective amount of the genetically modified immune cell of claim 139, wherein said cancer is selected from bladder cancer, bone cancer, brain cancer, breast cancer, cervical cancer, colon cancer, esophageal cancer, gastric cancer, glioma, head and neck cancer, kidney cancer, leukemia, acute myeloid leukemia (AML), multiple myeloma, liver cancer, lung cancer, lymphoma, melanoma, mesothelioma, medulloblastoma, ovarian cancer, pancreatic cancer, prostate cancer, rectal cancer, skin cancer, testicular cancer, tracheal cancer, and vulvar cancer.
